(19) United States
(12) Patent Application Publication
(10) Pub. No.: US 2014/0073548 A1
(43) Pub. Date: Mar. 13, 2014

(54) DETERGENT COMPOSITIONS CONTAINING BACILLUS SP. MANNANASE AND METHODS OF USE THEREOF

(71) Applicants: Brian E. Jones, Leidschendam (GB); Marc Kolkman, Oegstgeest (NL); Zhen Qian, Shanghai (CN); Brian Sogaard Laursen, Kalunbourg (DK); Karsten M. Kragh, Viby J (DK); Sina Pricelius, Arhus C (DE); Zheyong Yu, Shanghai (CN); Lilia Maria Babe, Emerald Hills, CA (US); Melodie Estabrook, Mountain View, CA (US); Ling Hua, Hockessin, DE (US)

(72) Inventors: Brian E. Jones, Leidschendam (GB); Marc Kolkman, Oegstgeest (NL); Zhen Qian, Shanghai (CN); Brian Sogaard Laursen, Kalunbourg (DK); Karsten M. Kragh, Viby J (DK); Sina Pricelius, Arhus C (DE); Zheyong Yu, Shanghai (CN); Lilia Maria Babe, Emerald Hills, CA (US); Melodie Estabrook, Mountain View, CA (US); Ling Hua, Hockessin, DE (US)

(73) Assignee: Danisco US Inc., Palo Alto, CA (US)

(21) Appl. No.: 14/114,720

(22) PCT Filed: Apr. 27, 2012
(86) PCT No.: PCT/US2012/035472

§ 371 Date: Oct. 29, 2013

(30) Foreign Application Priority Data
Apr. 29, 2011 (CN) PCT/CN2011/073559

(51) Int. Cl. C11D 3/386; C12N 9/24

(52) U.S. Cl. CPC C11D 3/38636; C12N 9/2491
USPC 510/218; 510/392; 510/393; 510/320; 510/515; 435/200; 435/188

(57) ABSTRACT

The present compositions and methods relate to an endo-B-mannanase cloned from a Bacillus sp., polynucleotides encoding the endo-B-mannanase, and methods of use thereof. Formulations containing the endo-β-mannanase are highly suitable for use as detergents.

### PRIORITY

The present application claims priority to International Application No. PCT/CN2011/073559, filed on Apr. 29, 2011, which are hereby incorporated by reference in their entirety.

### TECHNICAL FIELD

The present compositions and methods relate to an endo-β-mannanase cloned from a Bacillus sp., polynucleotides encoding the endo-β-mannanase, and methods of use thereof. Formulations containing the endo-β-mannanase are highly suitable for use as detergents.

### BACKGROUND

Current laundry detergent and fabric care compositions include a complex combination of active ingredients such as surfactants, enzymes (protease, amylase, mannanase, and/or cellulase), bleaching agents, a builder system, suds suppressors, soil-suspending agents, soil-release agents, optical brighteners, softening agents, dispersants, dye transfer inhibition compounds, abrasives, bactericides, and perfumes.

Mannanase enzymes, including endo-β-mannanases, have been employed in detergent cleaning compositions for the removal of gum stains by hydrolyzing mannans. A variety of mannans are found in nature. These include linear mannan, glucomannan, galactomannan, and glucogalactomannan. In each case, the polysaccharide contains a β-1,4-linked backbone of mannose residues that may be substituted up to 33% with glucose residues (Yeoman et al., Adv Appl Microbiol, Elsivier). In galactomannans or glucogalactomannnans, galactose residues are linked in alpha-1,6-linkages to the mannan backbone (Moreira and Filho, Appl Microbiol Biotechnol, 79:165, 2008). Therefore, hydrolysis of mannan to its component sugars requires endo-1,4-β-mannanases that hydrolyze the backbone linkages to generate short chain manno-oligosaccharides that are further degraded to monosaccharides by 1,4-β-mannosidases.

However, enzymes are often inhibited by surfactants and other components present in cleaning compositions, which interferes with their ability to remove stains. For instance, proteases present in laundry detergents may degrade mannanases before the removal of a gum stain occurs. In addition, mannanases may have a limited pH and/or temperature range at which they are active, which may make them unsuitable for certain formulations and washing conditions. Accordingly, the need exists for endo-β-mannanases that retain activity in the harsh environment of cleaning compositions.

### SUMMARY

The present compositions and methods relate to endo-β-mannanasel cloned from Bacillus sp. SWT81 (Bsp Man4). Formulations containing the endo-β-mannanase are highly suitable for use in detergents, food or feed.

In particular the present disclosure provides recombinant polypeptides comprising a catalytic domain of an endo-β-mannanase, wherein the catalytic domain is at least 85% (85%, 86%, 87%, 88%, 89%, 90, 91%, 92%, 93%, 94%, 95%, 96%, 97%, 98%, 99% or 100%) identical to the amino acid sequence of SEQ ID NO:9. The present disclosure also provides recombinant polypeptides comprising a mature form of an endo-β-mannanase, wherein the mature form is at least 80% (80%, 81%, 82%, 83%, 84%, 85%, 86%, 87%, 88%, 89%, 90, 91%, 92%, 93%, 94%, 95%, 96%, 97%, 98%, 99% or 100%) identical to the amino acid sequence of SEQ ID NO:8. In some embodiments, the polypeptide has measurable mannanase activity in the presence of detergent. In some embodiments, the polypeptide has measurable mannanase activity in the presence of a protease. In some embodiments, the polypeptide and the protease are both present at from about 0.1 to about 10.0 ppm. In some embodiments, the polypeptide retains greater than 70% mannanase activity at pH values of between 6 and 8.5. In some embodiments, the polypeptide has a pH optimum of about 6.5. In some embodiments, the polypeptide retains greater than 70% mannanase activity at a temperature range from 55° C. to 65° C. In some embodiments, the polypeptide has a temperature optimum of about 60° C. In some embodiments, the polypeptide is capable of hydrolyzing a substrate selected from the group consisting of chocolate ice cream, guar gum, locust bean gum, and combinations thereof. In some embodiments, wherein the amino acid sequence is at least 95% identical to one of the group consisting of SEQ ID NOS:6-14 and 30-49. In some embodiments, the polypeptide further comprises an amino-terminal extension of Ala-Gly-Lys. In some embodiments, the polypeptide further comprises a native or non-native signal peptide. In some embodiments, the polypeptide further comprises at least one carbohydrate-binding module. In other embodiments, the polypeptide does not comprise a carbohydrate-binding module.

Also provided by the present disclosure are detergent compositions comprising at least one recombinant polypeptide of the preceding paragraph. In some embodiments, the composition further comprises a surfactant. In some embodiments, the surfactant is selected from the group consisting of sodium dodecylbenzene sulfonate, sodium hydrogenated cocoate, sodium laureth sulfate, C12-14 pareth-7, C12-15 pareth-7, sodium C12-15 pareth sulfate, C14-15 pareth-4, and combinations thereof. In some preferred embodiments, the surfactant is an ionic surfactant. In some embodiments, the ionic surfactant is selected from the group consisting of an anionic surfactant, a cationic surfactant, a zwitterionic surfactant, and a combination thereof. In some preferred embodiments, the composition further comprises an enzyme selected from the group consisting proteases, proteases, peroxidases, cellulases, beta-glucanases, hemicellulases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-β-mannanases, exo-β-mannanases, pectin methylesterases, cellobiohydrolases, transglutaminases, and combinations thereof. In some embodiments, the combination comprises a protease and an amylase. In some embodiments, the detergent is selected from the group consisting of a laundry detergent, a fabric softening detergent, a dishwashing detergent, and a hard-surface cleaning detergent. In some embodiments, the detergent is in a form selected from the group consisting of a liquid, a powder, a granulated solid, and a tablet. In addition the present disclosure provides methods for hydrolyzing a mannan substrate present in a soil or stain on a surface, comprising: contacting the surface with the detergent composition to produce a clean surface. Also provided are methods of textile cleaning comprising: contacting a soiled textile with the detergent composition to produce a clean textile.

Moreover, the present disclosure provides isolated nucleic acids encoding the recombinant polypeptide of the preceding paragraphs. Also provided are expression vectors comprising the isolated nucleic acid in operable combination to a regulatory sequence. Additionally, host cells comprising the expression vector are provided. In some embodiments, the host cell is a bacterial cell or a fungal cell. The present disclosure further provides methods of producing an endo-β-mannanase, comprising: culturing the host cell in a culture medium, under suitable conditions to produce a culture comprising the endo-β-mannanase. In some embodiments, the methods further comprise removing the host cells from the culture by centrifugation, and removing debris of less than 10 kDa by filtration to produce an endo-β-mannanase-enriched supernatant. The present disclosure further provides methods for hydrolyzing a polysaccharide, comprising: contacting a polysaccharide comprising mannose with the supernatant to produce oligosaccharides comprising mannose. In some embodiments, the polysaccharide is selected from the group consisting of mannan, glucomannan, galactomannan, galactoglucomannan, and combinations thereof.

Also provided by the present disclosure are food or feed compositions having at least one recombinant polypeptide as described above, methods of preparing these compositions and uses of these compositions. This includes animal and/or human food or feed, which also includes fermented beverages.

These and other aspects of Bsp Man4 compositions and methods will be apparent from the following description.

### DESCRIPTION OF THE DRAWINGS

FIG. 1 provides a plasmid map of pZQ186 (aprE-Bsp Man4).

FIG. 2A shows the pH profile of Bsp Man4. FIG. 2B shows the pH profile for a benchmark endo-β-mannanase (Mannastar™).

FIG. 3A shows the temperature profile of Bsp Man4. FIG. 3B shows the temperature profile of a benchmark endo-β-mannanase (Mannastar™).

FIG. 4A shows the mannanase activity of various forms of Bsp Man4 at 30° C., for 30 min at pH 8.2. FIG. 4B shows the mannanase activity of various forms of Bsp Man4 at 50° C., for 10 min at pH 5.

FIG. 5A shows the cleaning performance of Bsp Man4 in Small & Mighty liquid detergent. FIG. 5B shows the cleaning performance of Bsp Man4 in OMO Color powder detergent.

FIG. 6A shows the cleaning performance of various forms of Bsp Man4 in the presence of a protease and an amylase in Small & Mighty liquid detergent. FIG. 6B shows the cleaning performance of various forms of Bsp Man4 in the presence of a protease and an amylase in OMO Color powder detergent.

FIG. 7A-C provides an alignment of the amino acid sequence of the mature form of Bsp Man4 (SEQ ID NO:8) with the sequences of other microbial mannanases (SEQ ID NOs:15-24). Table 7-1 lists the homologous mannanases by NCBI and SEQ ID NO.

FIG. 8 provides a phylogenetic tree for Bsp Man4.

FIG. 9 shows the predicted functional domains of Bsp Man4. The catalytic domain of Bsp Man4 (SEQ ID NO:9) corresponds to residues 11-306 of SEQ ID NO:8. The two predicted catalytic glutamic acid (E) residues are marked. Also shown are the two predicted carbohydrate-binding modules of Bsp Man4.

FIG. 10 provides the diagrams of protein domains for Bsp Man4 and Bsp Man4 C-terminal truncations.

FIG. 11A-D provides plasmid maps of pLL007 (aprE-Bsp Man4 1-350), pLL008 (aprE-Bsp Man4 1-475), pLL009 (aprE-Bsp Man4 1-675), and pLL010 (aprE-Bsp Man4 1-850).

FIG. 12 shows the pH profile of Bsp Man4v2

FIG. 13 shows the temperature profile of Bsp Man4v2

FIG. 14 shows the thermostability of Bsp Man4 and Bsp Man4v2

### DETAILED DESCRIPTION

### I. Introduction

Described are compositions and methods relating to endo-β-mannanase4 cloned from Bacillus sp SWT81 (Bsp Man4). The compositions and methods are based, in part, on the observation that recombinant Bsp Man4 has glycosyl hydrolase activity in the presence of detergent compositions. This feature of Bsp Man4 makes it well suited for use in a variety of cleaning applications, where the enzyme can hydrolyze mannans in the presence of surfactants and other components found in detergent compositions.

### II. Definitions

Prior to describing the present compositions and methods in detail, the following terms are defined for clarity. Terms and abbreviations not defined should be accorded their ordinary meaning as used in the art:

As used herein, a “mannan endo-1,4-β-mannosidase,” “endo-1,4-β-mannanase,” “endo-β-1,4-mannase,” “β-mannanase B,” “β-1,4-mannan 4-mannanohydrolase,” “endo-β-mannanase,” “β-D-mannanase,” “1,4-β-D-mannan mannanohydrolase,” or “endo-β-mannanase” (EC 3.2.1.78) refers to an enzyme capable of the hydrolysis of 1,4-β-D-mannosidic linkages in mannans, galactomannans and glucomannans. Endo-1,4-β-mannanases are members of several families of glycosyl hydrolases, including GH26 and GH5. In particular, endo-β-mannanases constitute a group of polysaccharases that degrade mannans and denote enzymes that are capable of cleaving polyose chains containing mannose units (i.e., are capable of cleaving glycosidic bonds in mannans, glucomannans, galactomannans and galactogluco-mannans). The “endo-β-mannanases” of the present disclosure may possess additional enzymatic activities (e.g., endo-1,4-β-glucanase, 1,4-(3-mannosidase, cellodextrinase activities, etc.).

As used herein, a “mannanase,” “mannosidic enzyme,” “mannolytic enzyme,” “mannanase enzyme,” “mannanase polypeptides,” or “mannanase proteins” refers to an enzyme, polypeptide, or protein exhibiting a mannan degrading capability. The mannanase enzyme may be, for example, an endo-β-mannanase, an exo-β-mannanase, or a glycosyl hydrolase. As used herein, mannanase activity may be determined according to any procedure known in the art (See, e.g., Lever, Anal. Biochem, 47:248, 1972; U.S. Pat. No. 6,602,842; and International Publication No. WO 95/35362A1).

As used herein, “mannans” are polysaccharides having a backbone composed of β-1,4-linked mannose; “glucomannans” are polysaccharides having a backbone of more or less regularly alternating β-1,4 linked mannose and glucose; “galactomannans” and “galactoglucomannans” are mannans and glucomannans with alpha-1,6 linked galactose sidebranches. These compounds may be acetylated. The degradation of galactomannans and galactoglucomannans is facilitated by full or partial removal of the galactose sidebranches. Further the degradation of the acetylated mannans, glucomannans, galactomannans and galactoglucomannans is facilitated by full or partial deacetylation. Acetyl groups can be removed by alkali or by mannan acetylesterases. The oligomers that are released from the mannanases or by a combination of mannanases and alpha-galactosidase and/or mannan acetyl esterases can be further degraded to release free maltose by β-mannosidase and/or β-glucosidase

As used herein, “catalytic activity” or “activity” describes quantitatively the conversion of a given substrate under defined reaction conditions. The term “residual activity” is defined as the ratio of the catalytic activity of the enzyme under a certain set of conditions to the catalytic activity under a different set of conditions. The term “specific activity” describes quantitatively the catalytic activity per amount of enzyme under defined reaction conditions.

As used herein, “pH-stability” describes the property of a protein to withstand a limited exposure to pH-values significantly deviating from the pH where its stability is optimal (e.g., more than one pH-unit above or below the pH-optimum, without losing its activity under conditions where its activity is measurable).

As used herein, the phrase “detergent stability” refers to the stability of a specified detergent composition component (such as a hydrolytic enzyme) in a detergent composition mixture.

As used herein, a “perhydrolase” is an enzyme capable of catalyzing a reaction that results in the formation of a peracid suitable for applications such as cleaning, bleaching, and disinfecting.

As used herein, the term “aqueous,” as used in the phrases “aqueous composition” and “aqueous environment,” refers to a composition that is made up of at least 50% water. An aqueous composition may contain at least 50% water, at least 60% water, at least 70% water, at least 80% water, at least 90% water, at least 95% water, at least 97% water, at least 99% water, or even at least 99% water.

As used herein, the term “surfactant” refers to any compound generally recognized in the art as having surface active qualities. Surfactants generally include anionic, cationic, nonionic, and zwitterionic compounds, which are further described, herein.

As used herein, “surface property” is used in reference to electrostatic charge, as well as properties such as the hydrophobicity and hydrophilicity exhibited by the surface of a protein.

The term “oxidation stability” refers to endo-β-mannanases of the present disclosure that retain a specified amount of enzymatic activity over a given period of time under conditions prevailing during the mannosidic, hydrolyzing, cleaning, or other process disclosed herein, for example while exposed to or contacted with bleaching agents or oxidizing agents. In some embodiments, the endo-β-mannanases retain at least about 50%, about 60%, about 70%, about 75%, about 80%, about 85%, about 90%, about 92%, about 95%, about 96%, about 97%, about 98%, or about 99% endo-β-mannanase activity after contact with a bleaching or oxidizing agent over a given time period, for example, at least about 1 minute, about 3 minutes, about 5 minutes, about 8 minutes, about 12 minutes, about 16 minutes, about 20 minutes, etc.

The term “chelator stability” refers to endo-β-mannanases of the present disclosure that retain a specified amount of enzymatic activity over a given period of time under conditions prevailing during the mannosidic, hydrolyzing, cleaning, or other process disclosed herein, for example while exposed to or contacted with chelating agents. In some embodiments, the endo-β-mannanases retain at least about 50%, about 60%, about 70%, about 75%, about 80%, about 85%, about 90%, about 92%, about 95%, about 96%, about 97%, about 98%, or about 99% endo-β-mannanase activity after contact with a chelating agent over a given time period, for example, at least about 10 minutes, about 20 minutes, about 40 minutes, about 60 minutes, about 100 minutes, etc.

The terms “thermal stability” and “thermostable” refer to endo-β-mannanases of the present disclosure that retain a specified amount of enzymatic activity after exposure to identified temperatures over a given period of time under conditions prevailing during the mannosidic, hydrolyzing, cleaning, or other process disclosed herein, for example, while exposed to altered temperatures. Altered temperatures include increased or decreased temperatures. In some embodiments, the endo-β-mannanases retain at least about 50%, about 60%, about 70%, about 75%, about 80%, about 85%, about 90%, about 92%, about 95%, about 96%, about 97%, about 98%, or about 99% endo-β-mannanase activity after exposure to altered temperatures over a given time period, for example, at least about 60 minutes, about 120 minutes, about 180 minutes, about 240 minutes, about 300 minutes, etc.

The term “cleaning activity” refers to the cleaning performance achieved by the endo-β-mannanase under conditions prevailing during the mannosidic, hydrolyzing, cleaning, or other process disclosed herein. In some embodiments, cleaning performance is determined by the application of various cleaning assays concerning enzyme sensitive stains, for example ice cream, ketchup, BBQ sauce, mayonnaise, chocolate milk, body lotion, locust bean gum, or guar gum as determined by various chromatographic, spectrophotometric or other quantitative methodologies after subjection of the stains to standard wash conditions. Exemplary assays include, but are not limited to those described in WO 99/34011, U.S. Pat. No. 6,605,458, and U.S. Pat. No. 6,566,114 (all of which are herein incorporated by reference), as well as those methods included in the Examples.

As used herein, the terms “clean surface” and “clean textile” refer to a surface or textile respectively that has a percent stain removal of at least 10%, preferably at least 15%, 20%, 25%, 30%, 35%, or 40% of a soiled surface or textile.

The term “cleaning effective amount” of an endo-β-mannanase refers to the quantity of endo-β-mannanase described hereinbefore that achieves a desired level of enzymatic activity in a specific cleaning composition. Such effective amounts are readily ascertained by one of ordinary skill in the art and are based on many factors, such as the particular endo-β-mannanase used, the cleaning application, the specific composition of the cleaning composition, and whether a liquid or dry (e.g., granular, bar) composition is required, etc.

The term “cleaning adjunct materials,” as used herein, means any liquid, solid or gaseous material selected for the particular type of cleaning composition desired and the form of the product (e.g., liquid, granule, powder, bar, paste, spray, tablet, gel, or foam composition), which materials are also preferably compatible with the endo-β-mannanase enzyme used in the composition. In some embodiments, granular compositions are in “compact” form, while in other embodiments, the liquid compositions are in a “concentrated” form.

As used herein, “cleaning compositions” and “cleaning formulations” refer to admixtures of chemical ingredients that find use in the removal of undesired compounds (e.g., soil or stains) from items to be cleaned, such as fabric, dishes, contact lenses, other solid surfaces, hair, skin, teeth, and the like. The composition or formulations may be in the form of a liquid, gel, granule, powder, or spray, depending on the surface, item or fabric to be cleaned, and the desired form of the composition or formulation.

As used herein, the terms “detergent composition” and “detergent formulation” refer to mixtures of chemical ingredients intended for use in a wash medium for the cleaning of soiled objects. Detergent compositions/formulations generally include at least one surfactant, and may optionally include hydrolytic enzymes, oxido-reductases, builders, bleaching agents, bleach activators, bluing agents and fluorescent dyes, caking inhibitors, masking agents, enzyme activators, antioxidants, and solubilizers.

As used herein, “laundry composition” or “laundry detergent” refers to all forms of compositions for cleaning textiles, including but not limited to granular and liquid forms. In some embodiments, the laundry composition is a composition that finds use in an electric clothes washer. It is not intended that the present disclosure be limited to any particular type or laundry composition. Indeed, the present disclosure finds use in cleaning many fabrics.

As used herein, “dishwashing composition” refers to all forms of compositions for cleaning dishware, including cutlery, including but not limited to granular and liquid forms. In some embodiments, the dishwashing composition is an “automatic dishwashing” composition that finds use in automatic dish washing machines. It is not intended that the present disclosure be limited to any particular type or dishware composition. Indeed, the present disclosure finds use in cleaning dishware (e.g., dishes including, but not limited to plates, cups, glasses, bowls, etc.) and cutlery (e.g., utensils including, but not limited to spoons, knives, forks, serving utensils, etc.) of any material, including but not limited to ceramics, plastics, metals, china, glass, acrylics, etc. The term “dishware” is used herein in reference to both dishes and cutlery.

As used herein, the term “bleaching” refers to the treatment of a material (e.g., fabric, laundry, pulp, etc.) or surface for a sufficient length of time and under appropriate pH and temperature conditions to effect a brightening (i.e., whitening) and/or cleaning of the material. Examples of chemicals suitable for bleaching include but are not limited to ClO₂, H₂O₂, peracids, NO₂, etc.

As used herein, “wash performance” of a variant endo-β-mannanase refers to the contribution of a variant endo-β-mannanase to washing that provides additional cleaning performance to the detergent without the addition of the variant endo-β-mannanase to the composition. Wash performance is compared under relevant washing conditions.

The term “relevant washing conditions” is used herein to indicate the conditions, particularly washing temperature, time, washing mechanics, sud concentration, type of detergent, and water hardness, actually used in households in a dish or laundry detergent market segment.

As used herein, the term “disinfecting” refers to the removal of contaminants from the surfaces, as well as the inhibition or killing of microbes on the surfaces of items. It is not intended that the present disclosure be limited to any particular surface, item, or contaminant(s) or microbes to be removed.

The “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts not exceeding about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, a preferred filler salt is sodium sulfate.

As used herein, the terms “textile” or “textile material” refer to woven fabrics, as well as staple fibers and filaments suitable for conversion to or use as yarns, woven, knit, and non-woven fabrics. The term encompasses yarns made from natural, as well as synthetic (e.g., manufactured) fibers.

As used herein, the terms “purified” and “isolated” refer to the physical separation of a subject molecule, such as Bsp Man4, from its native source (e.g., Bacillus sp.) or other molecules, such as proteins, nucleic acids, lipids, media components, and the like. Once purified or isolated, a subject molecule may represent at least 50%, and even at least 60%, at least 70%, at least 80%, at least 85%, at least 90%, at least 95%, or more, of the total amount of material in a sample (wt/wt).

As used herein, a “polypeptide” refers to a molecule comprising a plurality of amino acids linked through peptide bonds. The terms “polypeptide,” “peptide,” and “protein” are used interchangeably. Proteins maybe optionally be modified (e.g., glycosylated, phosphorylated, acylated, farnesylated, prenylated, sulfonated, pegylated, and the like) to add functionality. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The terms “polynucleotide” encompasses DNA, RNA, heteroduplexes, and synthetic molecules capable of encoding a polypeptide. Nucleic acids may be single-stranded or double-stranded, and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. Unless otherwise indicated, nucleic acid sequences are presented in a 5′-to-3′ orientation.

As used herein, the terms “wild-type” and “native” refer to polypeptides or polynucleotides that are found in nature.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues and may be defined by their level of primary amino acid sequence homology/identity with a parent polypeptide. Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent polypeptide.

Sequence identity may be determined using known programs such as BLAST, ALIGN, and CLUSTAL using standard parameters. (See, e.g., Altschul et al. [1990] J. Mol. Biol. 215:403-410; Henikoff et al. [1989] Proc. Natl. Acad. Sci. USA 89:10915; Karin et al. [1993] Proc. Natl. Acad. Sci. USA 90:5873; and Higgins et al. [1988] Gene 73:237-244). Software for performing BLAST analyses is publicly available through the National Center for Biotechnology Information. Databases may also be searched using FASTA (Pearson et al. [1988] Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution.

As used herein, a “variant polynucleotide” encodes a variant polypeptide, has a specified degree of homology/identity with a parent polynucleotide, or hybridized under stringent conditions to a parent polynucleotide or the complement, thereof. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent polynucleotide. Methods for determining percent identity are known in the art and described immediately above.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization” refers to the process by which a strand of nucleic acid joins with a complementary strand through base pairing, as known in the art.

As used herein, the phrase “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions can be based upon the salt or ionic strength conditions of hybridization and/or one or more stringency washes, e.g.,: 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used). As used herein, stringent conditions are defined as 50° C. and 0.2×SSC (1×SSC=0.15 M NaCl, 0.015 M sodium citrate, pH 7.0).

The phrases “substantially similar” and “substantially identical” in the context of at least two nucleic acids or polypeptides means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

As used herein, an “expression vector” refers to a DNA construct containing a DNA sequence that encodes a specified polypeptide and is operably linked to a suitable control sequence capable of effecting the expression of the polypeptides in a suitable host. Such control sequences include a promoter to effect transcription, an optional operator sequence to control such transcription, a sequence encoding suitable mRNA ribosome binding sites and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated by man such that they are not identical to related nucleic acids, polypeptides, and cells found in nature.

A “signal sequence” refers to a sequence of amino acids bound to the N-terminal portion of a polypeptide, and which facilitates the secretion of the mature form of the protein from the cell. The mature form of the extracellular protein lacks the signal sequence which is cleaved off during the secretion process.

The term “selective marker” or “selectable marker” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “regulatory element” as used herein refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “host cells” are generally prokaryotic or eukaryotic hosts which are transformed or transfected with vectors constructed using recombinant DNA techniques known in the art. Transformed host cells are capable of either replicating vectors encoding the protein variants or expressing the desired protein variant. In the case of vectors which encode the pre- or pro-form of the protein variant, such variants, when expressed, are typically secreted from the host cell into the host cell medium.

The term “introduced” in the context of inserting a nucleic acid sequence into a cell, means transformation, transduction or transfection. Means of transformation include protoplast transformation, calcium chloride precipitation, electroporation, naked DNA, and the like as known in the art. (See, Chang and Cohen [1979] Mol. Gen. Genet. 168:111-115; Smith et al. [1986] Appl. Env. Microbiol. 51:634; and the review article by Ferrari et al., in Harwood, Bacillus, Plenum Publishing Corporation, pp. 57-72, 1989).

The terms “selectable marker” or “selectable gene product” as used herein refer to the use of a gene, which encodes an enzymatic activity that confers resistance to an antibiotic or drug upon the cell in which the selectable marker is expressed.

Other technical and scientific terms have the same meaning as commonly understood by one of ordinary skill in the art to which this disclosure pertains (See, e.g., Singleton and Sainsbury, Dictionary of Microbiology and Molecular Biology, 2d Ed., John Wiley and Sons, NY 1994; and Hale and Marham, The Harper Collins Dictionary of Biology, Harper Perennial, NY 1991).

The singular terms “a,” “an,” and “the” include the plural reference unless the context clearly indicates otherwise.

As used herein in connection with a numerical value, the term “about” refers to a range of −10% to +10% of the numerical value. For instance, the phrase a “pH value of about 6” refers to pH values of from 5.4 to 6.6.

Headings are provided for convenience and should not be construed as limitations. The description included under one heading may apply to the specification as a whole.

### III. Bsp Man4 Polypeptides, Polynucleotides, Vectors, and Host Cells

A. Bsp Man4 Polypeptides

In one aspect, the present compositions and methods provide a recombinant Bsp Man4 endo-β-mannanase polypeptide, fragments thereof, or variants thereof. An exemplary Bsp Man4 polypeptide was recombinantly expressed from a polynucleotide obtained from Bacillus sp. The mature Bsp Man4 polypeptide has the amino acid sequence set forth as SEQ ID NO:8. Similar, substantially identical Bsp Man4 polypeptides may occur in nature, e.g., in other strains or isolates of Bacillus. These and other Bsp Man4 polypeptides are encompassed by the present compositions and methods. Bsp Man4 polypeptides of the present invention include truncated forms of Bsp Man4, including C-terminal truncations, that retain mannanase activity. Included amongst these polypeptides are the polypeptides as describes in the Examples and shown as SEQ ID NOs:6-14 and 30-49.

In some embodiments, the isolated Bsp Man4 polypeptide is a variant Bsp Man4 polypeptide having a specified degree of amino acid sequence identity to the exemplified Bsp Man4 polypeptide, e.g., at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO:8. Sequence identity can be determined by amino acid sequence alignment, e.g., using a program such as BLAST, ALIGN, or CLUSTAL, as described herein.

In some embodiments, the isolated Bsp Man4 polypeptide is a variant Bsp Man4 polypeptide having a specified degree of amino acid sequence identity to the exemplified Bsp Man4 polypeptide, e.g., at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of any one of SEQ ID NOs:6-14 or 30-49. Sequence identity can be determined by amino acid sequence alignment, e.g., using a program such as BLAST, ALIGN, or CLUSTAL, as described herein.

In certain embodiments, the Bsp Man4 polypeptides are produced recombinantly, while in others the Bsp Man4 polypeptides are produced synthetically, or are purified from a native source (Bacillus sp.).

In certain other embodiments, the isolated Bsp Man4 polypeptide includes substitutions that do not substantially affect the structure and/or function of the polypeptide. Exemplary substitutions are conservative mutations, as summarized in Table I.

TABLE I
Amino Acid Substitutions

| Original Residue | Code | Acceptable Substitutions |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Substitutions involving naturally occurring amino acids are generally made by mutating a nucleic acid encoding a recombinant Bsp Man4 polypeptide, and then expressing the variant polypeptide in an organism. Substitutions involving non-naturally occurring amino acids or chemical modifications to amino acids are generally made by chemically modifying a recombinant Bsp Man4 polypeptide after it has been synthesized by an organism.

In some embodiments, variant isolated Bsp Man4 polypeptides are substantially identical to SEQ ID NO:8, meaning that they do not include amino acid substitutions, insertions, or deletions that do not significantly affect the structure, function, or expression of the polypeptide. Such variant isolated Bsp Man4 polypeptides include those designed only to circumvent the present description.

In some embodiments, the isolated Bsp Man4 polypeptide (including a variant thereof) has 1,4-β-D-mannosidic hydrolase activity, which includes mannanase, endo-1,4-(3-D-mannanase, exo-1,4-β-D-mannanasegalactomannanase, and/or glucomannanase activity. 1,4-β-D-mannosidic hydrolase activity can be determined and measured using the assays described herein, or by other assays known in the art. In some embodiments, the isolated Bsp Man4 polypeptide has activity in the presence of a detergent composition.

Bsp Man4 polypeptides include fragments of “full-length” Bsp Man4 polypeptides that retain 1,4-β-D-mannosidic hydrolase activity. Such fragments preferably retain the active site of the full-length polypeptides but may have deletions of non-critical amino acid residues. The activity of fragments can readily be determined using the assays described, herein, or by other assays known in the art. In some embodiments, the fragments of Bsp Man4 polypeptides retain 1,4-β-D-mannosidic hydrolase activity in the presence of a detergent composition. In some embodiments, the Bsp Man4 polypeptides comprise the catalytic domain of Bsp Man4 (SEQ ID NO:9), or a catalytic domain that has at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO:9.

In some embodiments, the Bsp Man4 amino acid sequences and derivatives are produced as a N- and/or C-terminal fusion protein, for example to aid in extraction, detection and/or purification and/or to add functional properties to the Bsp Man4 polypeptides. Examples of fusion protein partners include, but are not limited to, glutathione-S-transferase (GST), 6×His, GAL4 (DNA binding and/or transcriptional activation domains), FLAG, MYC, BCE103 (WO 2010/044786), or other tags well known to anyone skilled in the art. In some embodiments, a proteolytic cleavage site is provided between the fusion protein partner and the protein sequence of interest to allow removal of fusion protein sequences. Preferably, the fusion protein does not hinder the activity of the isolated Bsp Man4 polypeptide.

In some embodiments, the isolated Bsp Man4 polypeptide is fused to a functional domain including a leader peptide, propeptide, one or more binding domains (modules) and/or catalytic domain. Suitable binding domains include, but are not limited to, carbohydrate-binding modules (e.g., CBM) of various specificities, providing increased affinity to carbohydrate components present during the application of the isolated Bsp Man4 polypeptide. As described herein, the CBM and catalytic domain of the Bsp Man4 polypeptide are operably linked.

A carbohydrate-binding module (CBM) is defined as a contiguous amino acid sequence within a carbohydrate-active enzyme with a discreet fold having carbohydrate-binding activity. A few exceptions are CBMs in cellulosomal scaffoldin proteins and rare instances of independent putative CBMs. The requirement of CBMs existing as modules within larger enzymes sets this class of carbohydrate-binding protein apart from other non-catalytic sugar binding proteins such as lectins and sugar transport proteins. CBMs were previously classified as cellulose-binding domains (CBDs) based on the initial discovery of several modules that bound cellulose (Tomme et al., Eur J Biochem, 170:575-581, 1988; and Gilkes et al., J Biol Chem, 263:10401-10407, 1988). However, additional modules in carbohydrate-active enzymes are continually being found that bind carbohydrates other than cellulose yet otherwise meet the CBM criteria, hence the need to reclassify these polypeptides using more inclusive terminology. Previous classification of cellulose-binding domains was based on amino acid similarity. Groupings of CBDs were called “Types” and numbered with roman numerals (e.g. Type I or Type II CBDs). In keeping with the glycoside hydrolase classification, these groupings are now called families and numbered with Arabic numerals. Families 1 to 13 are the same as Types Ito XIII (Tomme et al., in Enzymatic Degradation of Insoluble Polysaccharides (Saddler, J. N. & Penner, M., eds.), Cellulose-binding domains: classification and properties. pp. 142-163, American Chemical Society, Washington, 1995). A detailed review on the structure and binding modes of CBMs can be found in (Boraston et al., Biochem J, 382:769-81, 2004). The family classification of CBMs is expected to: aid in the identification of CBMs, in some cases, predict binding specificity, aid in identifying functional residues, reveal evolutionary relationships and possibly be predictive of polypeptide folds. Because the fold of proteins is better conserved than their sequences, some of the CBM families can be grouped into superfamilies or clans. The current CBM families are 1-63. CBMs/CBDs have also been found in algae, e.g., the red alga Porphyra purpurea as a non-hydrolytic polysaccharide-binding protein. However, most of the CBDs are from cellullases and xylanases. CBDs are found at the N- and C-termini of proteins or are internal. Enzyme hybrids are known in the art (See e.g., WO 90/00609 and WO 95/16782) and may be prepared by transforming into a host cell a DNA construct comprising at least a fragment of DNA encoding the cellulose-binding domain ligated, with or without a linker, to a DNA sequence encoding a disclosed Bsp Man4 polypeptide and growing the host cell to express the fused gene. Enzyme hybrids may be described by the following formula:

CBM-MR-X or X-MR-CBM

In the above formula, the CBM is the N-terminal or the C-terminal region of an amino acid sequence corresponding to at least the carbohydrate-binding module; MR is the middle region (the linker), and may be a bond, or a short linking group preferably of from about 2 to about 100 carbon atoms, more preferably of from 2 to 40 carbon atoms; or is preferably from about 2 to about 100 amino acids, more preferably from 2 to 40 amino acids; and X is an N-terminal or C-terminal region of a disclosed Bsp Man4 polypeptide having mannanase catalytic activity. In addition, a mannanase may contain more than one CBM or other module(s)/domain(s) of non-glycolytic function. The terms “module” and “domain” are used interchangeably in the present disclosure.

Suitable enzymatically active domains possess an activity that supports the action of the isolated Bsp Man4 polypeptide in producing the desired product. Non-limiting examples of catalytic domains include: cellulases, hemicellulases such as xylanase, exo-mannanases, glucanases, arabinases, galactosidases, pectinases, and/or other activities such as proteases, lipases, acid phosphatases and/or others or functional fragments thereof. Fusion proteins are optionally linked to the isolated Bsp Man4 polypeptide through a linker sequence that simply joins the Bsp Man4 polypeptide and the fusion domain without significantly affecting the properties of either component, or the linker optionally has a functional importance for the intended application.

Alternatively, the isolated Bsp Man4 polypeptides described herein are used in conjunction with one or more additional proteins of interest. Non-limiting examples of proteins of interest include: hemicellulases, exo-β-mannanases, alpha-galactosidases, beta-galactosidases, lactases, beta-glucanases, endo-beta-1,4-glucanases, cellulases, xylosidases, xylanases, xyloglucanases, xylan acetyl-esterases, galactanases, exo-mannanases, pectinases, pectin lyases, pectinesterases, polygalacturonases, arabinases, rhamnogalacturonases, laccases, reductases, oxidases, phenoloxidases, ligninases, proteases, amylases, phosphatases, lipolytic enzymes, cutinases and/or other enzymes.

In other embodiments, the isolated Bsp Man4 polypeptide is fused to a signal peptide for directing the extracellular secretion of the isolated Bsp Man4 polypeptide. For example, in certain embodiments, the signal peptide is the native Bsp Man4 signal peptide. In other embodiments, the signal peptide is a non-native signal peptide such as the B. subtilis AprE signal peptide. In some embodiments, the isolated Bsp Man4 polypeptide has an N-terminal extension of Ala-Gly-Lys between the mature form and the signal peptide.

In some embodiments, the isolated Bsp Man4 polypeptide is expressed in a heterologous organism, i.e., an organism other than Bacillus agaradhaerens. Exemplary heterologous organisms are Gram(+) bacteria such as Bacillus subtilis, Bacillus lichenifonnis, Bacillus lentus, Bacillus brevis, Geobacillus (formerly Bacillus) stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans, or Streptomyces murinus; Gram(−) bacteria such as Escherichia coli.; yeast such as Saccharomyces spp. or Schizosaccharomyces spp., e.g. Saccharomyces cerevisiae; and filamentous fungi such as Aspergillus spp., e.g., Aspergillus oryzae or Aspergillus niger, and Trichoderma reesei. Methods from transforming nucleic acids into these organisms are well known in the art. A suitable procedure for transformation of Aspergillus host cells is described in EP 238 023.

In particular embodiments, the isolated Bsp Man4 polypeptide is expressed in a heterologous organism as a secreted polypeptide, in which case, the compositions and method encompass a method for expressing a Bsp Man4 polypeptide as a secreted polypeptide in a heterologous organism.

B. Bsp Man4 Polynucleotides

Another aspect of the compositions and methods is a polynucleotide that encodes an isolated Bsp Man4 polypeptide (including variants and fragments, thereof), provided in the context of an expression vector for directing the expression of a Bsp Man4 polypeptide in a heterologous organism, such as those identified, herein. The polynucleotide that encodes a Bsp Man4 polypeptide may be operably-linked to regulatory elements (e.g., a promoter, terminator, enhancer, and the like) to assist in expressing the encoded polypeptides.

An exemplary polynucleotide sequence encoding a Bsp Man4 polypeptide has the nucleotide sequence of SEQ ID NO:1. Similar, including substantially identical, polynucleotides encoding Bsp Man4 polypeptides and variants may occur in nature, e.g., in other strains or isolates of Bacillus. In view of the degeneracy of the genetic code, it will be appreciated that polynucleotides having different nucleotide sequences may encode the same Bsp Man4 polypeptides, variants, or fragments.

In some embodiments, polynucleotides encoding Bsp Man4 polypeptides have a specified degree of amino acid sequence identity to the exemplified polynucleotide encoding a Bsp Man4 polypeptide, e.g., at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO:8. In some embodiments, the polynucleotides encode Bsp Man4 polypeptides comprising the catalytic domain of Bsp Man4 (SEQ ID NO:9), or a catalytic domain that has at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO:9. Homology can be determined by amino acid sequence alignment, e.g., using a program such as BLAST, ALIGN, or CLUSTAL, as described herein.

In some embodiments, the polynucleotide that encodes a Bsp Man4 polypeptide is fused in frame behind (i.e., downstream of) a coding sequence for a signal peptide for directing the extracellular secretion of a Bsp Man4 polypeptide. Heterologous signal sequences include those from bacterial cellulase genes. Expression vectors may be provided in a heterologous host cell suitable for expressing a Bsp Man4 polypeptide, or suitable for propagating the expression vector prior to introducing it into a suitable host cell.

In some embodiments, polynucleotides encoding Bsp Man4 polypeptides hybridize to the exemplary polynucleotide of SEQ ID NO:1 (or the complement thereof) under specified hybridization conditions. Exemplary conditions are stringent condition and highly stringent conditions, which are described, herein.

Bsp Man4 polynucleotides may be naturally occurring or synthetic (i.e., man-made), and may be codon-optimized for expression in a different host, mutated to introduce cloning sites, or otherwise altered to add functionality.

C. Bsp Man4 Vectors and Host Cells

In order to produce a disclosed Bsp Man4 polypeptide, the DNA encoding the polypeptide can be chemically synthesized from published sequences or obtained directly from host cells harboring the gene (e.g., by cDNA library screening or PCR amplification). In some embodiments, the Bsp Man4 polynucleotide is included in an expression cassette and/or cloned into a suitable expression vector by standard molecular cloning techniques. Such expression cassettes or vectors contain sequences that assist initiation and termination of transcription (e.g., promoters and terminators), and generally contain a selectable marker.

The expression cassette or vector is introduced in a suitable expression host cell, which then expresses the corresponding Bsp Man4 polynucleotide. Particularly suitable expression hosts are bacterial expression host genera including Escherichia (e.g., Escherichia coli), Pseudomonas (e.g., P. fluorescens or P. stutzerei), Proteus (e.g., Proteus mirabilis), Ralstonia (e.g., Ralstonia eutropha), Streptomyces, Staphylococcus (e.g., S. carnosus), Lactococcus (e.g., L. lactis), or Bacillus (subtilis, megaterium, lichenifonnis, etc.). Also particularly suitable are yeast expression hosts such as Saccharomyces cerevisiae, Schizosaccharomyces pombe, Yarrowia lipolytica, Hansenula polymorpha, Kluyveromyces lactis or Pichia pastoris. Especially suited are fungal expression hosts such as Aspergillus niger, Chrysosporium lucknowense, Aspergillus (e.g., A. oryzae, A. niger, A. nidulans, etc.) or Trichoderma reesei. Also suited are mammalian expression hosts such as mouse (e.g., NS0), Chinese Hamster Ovary (CHO) or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing the Bsp Man4 polypeptide.

Promoters and/or signal sequences associated with secreted proteins in a particular host of interest are candidates for use in the heterologous production and secretion of endo-β-mannanases in that host or in other hosts. As an example, in filamentous fungal systems, the promoters that drive the genes for cellobiohydrolase I (cbh1), glucoamylase A (glaA), TAKA-amylase (amyA), xylanase (exlA), the gpd-promoter cbh1, cbhll, endoglucanase genes EGI-EGV, Cel61B, Cel74A, eg11-eg15, gpd promoter, Pgk1, pki1, EF-1alpha, tef1, cDNA1 and hex1 are particularly suitable and can be derived from a number of different organisms (e.g., A. niger, T. reesei, A. oryzae, A. awamori and A. nidulans). In some embodiments, the Bsp Man4 polynucleotide is recombinantly associated with a polynucleotide encoding a suitable homologous or heterologous signal sequence that leads to secretion of the Bsp Man4 polypeptide into the extracellular (or periplasmic) space, thereby allowing direct detection of enzyme activity in the cell supernatant (or periplasmic space or lysate). Particularly suitable signal sequences for Escherichia coli, other Gram negative bacteria and other organisms known in the art include those that drive expression of the HlyA, DsbA, Pbp, PhoA, Pe1B, OmpA, OmpT or M13 phage Gill genes. For Bacillus subtilis, Gram-positive organisms and other organisms known in the art, particularly suitable signal sequences further include those that drive expression of the AprE, NprB, Mpr, AmyA, AmyE, Blac, SacB, and for S. cerevisiae or other yeast, include the killer toxin, Bar1, Suc2, Mating factor alpha, Inu1A or Ggp1p signal sequence. Signal sequences can be cleaved by a number of signal peptidases, thus removing them from the rest of the expressed protein. In some embodiments, the rest of the Bsp Man4 polypeptide is expressed alone or as a fusion with other peptides, tags or proteins located at the N- or C-terminus (e.g., 6×His, HA or FLAG tags). Suitable fusions include tags, peptides or proteins that facilitate affinity purification or detection (e.g., BCE103, 6×His, HA, chitin binding protein, thioredoxin or FLAG tags), as well as those that facilitate expression, secretion or processing of the target endo-β-mannanase. Suitable processing sites include enterokinase, STE13, Kex2 or other protease cleavage sites for cleavage in vivo or in vitro.

Bsp Man4 polynucleotides are introduced into expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

Alternatively, the Bsp Man4 polypeptides are expressed intracellularly. Optionally, after intracellular expression of the enzyme variants, or secretion into the periplasmic space using signal sequences such as those mentioned above, a permeabilisation or lysis step can be used to release the Bsp Man4 polypeptide into the supernatant. The disruption of the membrane barrier is effected by the use of mechanical means such as ultrasonic waves, pressure treatment (French press), cavitation or the use of membrane-digesting enzymes such as lysozyme or enzyme mixtures. As a further alternative, the polynucleotides encoding the Bsp Man4 polypeptide are expressed by use of a suitable cell-free expression system. In cell-free systems, the polynucleotide of interest is typically transcribed with the assistance of a promoter, but ligation to form a circular expression vector is optional. In other embodiments, RNA is exogenously added or generated without transcription and translated in cell free systems.

### IV. Activities of Bsp Man4

The isolated Bsp Man4 polypeptides disclosed herein may have enzymatic activity over a broad range of pH conditions. In certain embodiments the disclosed Bsp Man4 polypeptides have enzymatic activity from about pH 4.0 to about pH 11.5. In preferred embodiments, the Bsp Man4 polypeptides have substantial enzymatic activity from about pH 6.0 to about pH 8.5. It should be noted that the pH values described herein may vary by ±0.2. For example a pH value of 8.0 could vary from pH 7.8 to pH 8.2.

The isolated Bsp Man4 polypeptides disclosed herein may have enzymatic activity over a wide range of temperatures, e.g., from 35° C. or lower to about 75° C. In certain embodiments, the Bsp Man4 polypeptides have substantial enzymatic activity at a temperature range of about 55° C. to about 65° C. It should be noted that the temperature values described herein may vary by ±0.2° C. For example a temperature of 50° C. could vary from 49.8° C. to 50.2° C.

As shown in Example 5, the Bsp Man4 polypeptide had cleaning performance against locust bean gum and guar gum in the presence of proteases. Moreover, Bsp Man4 showed hydrolysis activity against exemplary gum stained material, in the presence of both powder and liquid detergent. Accordingly, in certain embodiments, any of the isolated Bsp Man4 polypeptides described herein may hydrolyze mannan substrates that include, but are not limited to, locust bean gum, guar gum, and combinations thereof.

### V. Detergent Compositions Comprising a Bsp Man4 Polypeptide

An aspect of the compositions and methods disclosed herein is a detergent composition comprising an isolated Bsp Man4 polypeptide (including variants or fragments, thereof) and methods for using such compositions in cleaning applications. Cleaning applications include, but are not limited to, laundry or textile cleaning, laundry or textile softening, dishwashing (manual and automatic), stain pre-treatment, and the like. Particular applications are those where mannans (e.g., locust bean gum, guar gum, etc.) are a component of the soils or stains to be removed. Detergent compositions typically include an effective amount of any of the Bsp Man4 polypeptides described herein, e.g., at least 0.0001 weight percent, from about 0.0001 to about 1, from about 0.001 to about 0.5, from about 0.01 to about 0.1 weight percent, or even from about 0.1 to about 1 weight percent, or more. An effective amount of a Bsp Man4 polypeptide in the detergent composition results in the Bsp Man4 polypeptide having enzymatic activity sufficient to hydrolyze a mannan-containing substrate, such as locust bean gum, guar gum, or combinations thereof.

Additionally, detergent compositions having a concentration from about 0.4 g/L to about 2.2 g/L, from about 0.4 g/L to about 2.0 g/L, from about 0.4 g/L to about 1.7 g/L, from about 0.4 g/L to about 1.5 g/L, from about 0.4 g/L to about 1 g/L, from about 0.4 g/L to about 0.8 g/L, or from about 0.4 g/L to about 0.5 g/L may be mixed with an effective amount of an isolated Bsp Man4 polypeptide. The detergent composition may also be present at a concentration of about 0.4 ml/L to about 2.6 ml/L, from about 0.4 ml/L to about 2.0 ml/L, from about 0.4 ml/L to about 1.5 m/L, from about 0.4 ml/L to about 1 ml/L, from about 0.4 ml/L to about 0.8 ml/L, or from about 0.4 ml/L to about 0.5 ml/L.

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated. In the exemplified detergent compositions, the enzymes levels are expressed by pure enzyme by weight of the total composition and unless otherwise specified, the detergent ingredients are expressed by weight of the total compositions.

In some embodiments, the detergent composition comprises one or more surfactants, which may be non-ionic, semi-polar, anionic, cationic, zwitterionic, or combinations and mixtures thereof. The surfactants are typically present at a level of from about 0.1% to 60% by weight. Exemplary surfactants include but are not limited to sodium dodecylbenzene sulfonate, C12-14 pareth-7, C12-15 pareth-7, sodium C12-15 pareth sulfate, C14-15 pareth-4, sodium laureth sulfate (e.g., Steol CS-370), sodium hydrogenated cocoate, C12 ethoxylates (Alfonic 1012-6, Hetoxol LA7, Hetoxol LA4), sodium alkyl benzene sulfonates (e.g., Nacconol 90G), and combinations and mixtures thereof.

Anionic surfactants that may be used with the detergent compositions described herein include but are not limited to linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. It may also contain 0-40% of nonionic surfactant such as alcohol ethoxylate (AEO or AE), carboxylated alcohol ethoxylates, nonylphenol ethoxylate, alkylpolyglycoside, alkyldimethylamine oxide, ethoxylated fatty acid monoethanolamide, fatty acid monoethanolamide, polyhydroxy alkyl fatty acid amide (e.g., as described in WO 92/06154), and combinations and mixtures thereof.

Nonionic surfactants that may be used with the detergent compositions described herein include but are not limited to polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p-tert-octylphenols or octylphenyl-ethylene oxide condensates (e.g., NONIDET P40), ethylene oxide condensates with fatty alcohols (e.g., LUBROL), polyoxyethylene nonylphenols, polyalkylene glycols (SYNPERONIC F108), sugar-based surfactants (e.g., glycopyranosides, thioglycopyranosides), and combinations and mixtures thereof.

The detergent compositions disclosed herein may have mixtures that include, but are not limited to 5-15% anionic surfactants, <5% nonionic surfactants, cationic surfactants, phosphonates, soap, enzymes, perfume, butylphenyl methylptopionate, geraniol, zeolite, polycarboxylates, hexyl cinnamal, limonene, cationic surfactants, citronellol, and benzisothiazolinone.

Detergent compositions may additionally include one or more detergent builders or builder systems, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, a tarnish inhibitor, an optical brightener, a fabric conditioner, and a perfume. The detergent compositions may also include enzymes, including but not limited to proteases, amylases, cellulases, lipases, pectin degrading enzymes, xyloglucanases, or additional carboxylic ester hydrolases. The pH of the detergent compositions should be neutral to basic, as described herein.

In some embodiments incorporating at least one builder, the detergent compositions comprise at least about 1%, from about 3% to about 60% or even from about 5% to about 40% builder by weight of the cleaning composition. Builders may include, but are not limited to, the alkali metals, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1, 3, 5-trihydroxy benzene-2,4,6-trisulphonic acid, and carboxymethyloxysuccinic acid, the various alkali metals, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present disclosure.

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP 2 100 949).

As indicated herein, in some embodiments, the cleaning compositions described herein further comprise adjunct materials including, but not limited to surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See, e.g., U.S. Pat. Nos. 6,610,642; 6,605,458; 5,705,464; 5,710,115; 5,698,504; 5,695,679; 5,686,014; and 5,646,101; all of which are incorporated herein by reference). Embodiments of specific cleaning composition materials are exemplified in detail below. In embodiments in which the cleaning adjunct materials are not compatible with the Bsp Man4 variants in the cleaning compositions, suitable methods of keeping the cleaning adjunct materials and the endo-β-mannanase(s) separated (i.e., not in contact with each other), until combination of the two components is appropriate, are used. Such separation methods include any suitable method known in the art (e.g., gelcaps, encapsulation, tablets, physical separation, etc.).

The cleaning compositions described herein are advantageously employed for example, in laundry applications, hard surface cleaning, dishwashing applications, as well as cosmetic applications such as dentures, teeth, hair, and skin. In addition, due to the unique advantages of increased effectiveness in lower temperature solutions, the Bsp Man4 enzymes described herein are ideally suited for laundry and fabric softening applications. Furthermore, the Bsp Man4 enzymes may find use in granular and liquid compositions.

The isolated Bsp Man4 polypeptides described herein may also find use cleaning in additive products. In some embodiments, low temperature solution cleaning applications find use. In some embodiments, the present disclosure provides cleaning additive products including at least one disclosed Bsp Man4 polypeptide is ideally suited for inclusion in a wash process when additional bleaching effectiveness is desired. Such instances include, but are not limited to low temperature solution cleaning applications. In some embodiments, the additive product is in its simplest form, one or more endo-β-mannanases. In some embodiments, the additive is packaged in dosage form for addition to a cleaning process. In some embodiments, the additive is packaged in dosage form for addition to a cleaning process where a source of peroxygen is employed and increased bleaching effectiveness is desired. Any suitable single dosage unit form finds use with the present disclosure, including but not limited to pills, tablets, gelcaps, or other single dosage units such as pre-measured powders or liquids. In some embodiments, filler(s) or carrier material(s) are included to increase the volume of such compositions. Suitable filler or carrier materials include, but are not limited to various salts of sulfate, carbonate, and silicate as well as talc, clay, and the like. Suitable filler or carrier materials for liquid compositions include, but are not limited to water or low molecular weight primary and secondary alcohols including polyols and diols. Examples of such alcohols include, but are not limited to methanol, ethanol, propanol, and isopropanol. In some embodiments, the compositions contain from about 5% to about 90% of such materials. Acidic fillers find use to reduce pH. Alternatively, in some embodiments, the cleaning additive includes adjunct ingredients, as described more fully below.

The present cleaning compositions and cleaning additives require an effective amount of at least one of the Bsp Man4 polypeptides described herein, alone or in combination with other endo-β-mannanases and/or additional enzymes. In certain embodiments, the additional enzymes include, but are not limited to, at least one enzyme selected from proteases, peroxidases, cellulases (endoglucanases), beta-glucanases, hemicellulases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-β-mannanases, exo-β-mannanases, pectin methylesterases, cellobiohydrolases, transglutaminases, and mixtures thereof.

The required level of enzyme is achieved by the addition of one or more disclosed Bsp Man4 polypeptide. Typically the present cleaning compositions will comprise at least about 0.0001 weight percent, from about 0.0001 to about 10, from about 0.001 to about 1, or even from about 0.01 to about 0.1 weight percent of at least one of the disclosed Bsp Man4 polypeptides.

The cleaning compositions herein are typically formulated such that, during use in aqueous cleaning operations, the wash water will have a pH of from about 3.0 to about 11.0. Liquid product formulations are typically formulated to have a neat pH from about 5.0 to about 9.0. Granular laundry products are typically formulated to have a pH from about 8.0 to about 11.0. Techniques for controlling pH at recommended usage levels include the use of buffers, alkalis, acids, etc., and are well known to those skilled in the art.

Suitable low pH cleaning compositions typically have a neat pH of from about 3.0 to about 5.0, or even a neat pH of from 3.5 to 4.5. Low pH cleaning compositions are typically free of surfactants that hydrolyze in such a pH environment. Such surfactants include sodium alkyl sulfate surfactants that comprise at least one ethylene oxide moiety or even from about 1 to about 16 moles of ethylene oxide. Such cleaning compositions typically comprise a sufficient amount of a pH modifier, such as sodium hydroxide, monoethanolamine, or hydrochloric acid, to provide such cleaning composition with a neat pH of from about 3.0 to about 5.0. Such compositions typically comprise at least one acid stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20° C., unless otherwise indicated.

Suitable high pH cleaning compositions typically have a neat pH of from about 9.0 to about 11.0, or even a net pH of from 9.5 to 10.5. Such cleaning compositions typically comprise a sufficient amount of a pH modifier, such as sodium hydroxide, monoethanolamine, or hydrochloric acid, to provide such cleaning composition with a neat pH of from about 9.0 to about 11.0. Such compositions typically comprise at least one base-stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20° C., unless otherwise indicated.

In some embodiments, when the Bsp Man4 polypeptide is employed in a granular composition or in a liquid, it is desirable for the Bsp Man4 polypeptide to be in the form of an encapsulated particle to protect the Bsp Man4 polypeptide from other components of the granular composition during storage. In addition, encapsulation is also a means of controlling the availability of the Bsp Man4 polypeptide during the cleaning process. In some embodiments, encapsulation enhances the performance of the Bsp Man4 polypeptide and/or additional enzymes. In this regard, the Bsp Man4 polypeptides of the present disclosure are encapsulated with any suitable encapsulating material known in the art. In some embodiments, the encapsulating material typically encapsulates at least part of the catalyst for the Bsp Man4 polypeptides described herein. Typically, the encapsulating material is water-soluble and/or water-dispersible. In some embodiments, the encapsulating material has a glass transition temperature (Tg) of 0° C. or higher. Glass transition temperature is described in more detail in the PCT application WO 97/11151. The encapsulating material is typically selected from consisting of carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See, e.g., EP 0 922 499; U.S. Pat. No. 4,977,252; U.S. Pat. No. 5,354,559; and U.S. Pat. No. 5,935,826). In some embodiments, the encapsulating material is a microsphere made from plastic such as thermoplastics, acrylonitrile, methacrylonitrile, polyacrylonitrile, polymethacrylonitrile, and mixtures thereof; commercially available microspheres that find use include, but are not limited to those supplied by EXPANCEL® (Stockviksverken, Sweden), and PM 6545, PM 6550, PM 7220, PM 7228, EXTENDOSPHERES®, LUXSIL®, Q-CEL®, and SPHERICEL® (PQ Corp., Valley Forge, Pa.).

The term “granular composition” refers to a conglomeration of discrete solid, macroscopic particles. Powders are a special class of granular material due to their small particle size, which makes them more cohesive and more easily suspended.

In using detergent compositions that include Bsp Man4 in cleaning applications, the fabrics, textiles, dishes, or other surfaces to be cleaned are incubated in the presence of the Bsp Man4 detergent composition for a time sufficient to allow Bsp Man4 to hydrolyze mannan substrates including, but not limited to, locust bean gum, guar gum, and combinations thereof present in soil or stains, and then typically rinsed with water or another aqueous solvent to remove the Bsp Man4 detergent composition along with hydrolyzed mannans.

As described herein, the Bsp Man4 polypeptides find particular use in the cleaning industry, including, but not limited to laundry and dish detergents. These applications place enzymes under various environmental stresses. The Bsp Man4 polypeptides may provide advantages over many currently used enzymes, due to their stability under various conditions.

Indeed, there are a variety of wash conditions including varying detergent formulations, wash water volumes, wash water temperatures, and lengths of wash time, to which endo-β-mannanases involved in washing are exposed. In addition, detergent formulations used in different geographical areas have different concentrations of their relevant components present in the wash water. For example, European detergents typically have about 4500-5000 ppm of detergent components in the wash water, while Japanese detergents typically have approximately 667 ppm of detergent components in the wash water. In North America, particularly the United States, detergents typically have about 975 ppm of detergent components present in the wash water.

A low detergent concentration system includes detergents where less than about 800 ppm of the detergent components are present in the wash water. Japanese detergents are typically considered low detergent concentration system as they have approximately 667 ppm of detergent components present in the wash water.

A medium detergent concentration includes detergents where between about 800 ppm and about 2000 ppm of the detergent components are present in the wash water. North American detergents are generally considered to be medium detergent concentration systems as they have approximately 975 ppm of detergent components present in the wash water. Brazil typically has approximately 1500 ppm of detergent components present in the wash water.

A high detergent concentration system includes detergents where greater than about 2000 ppm of the detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1500 ppm to 6000 ppm of detergent components in the wash water. As mentioned above, Brazil typically has approximately 1500 ppm of detergent components present in the wash water. However, other high suds phosphate builder detergent geographies, not limited to other Latin American countries, may have high detergent concentration systems up to about 6000 ppm of detergent components present in the wash water.

In light of the foregoing, it is evident that concentrations of detergent compositions in typical wash solutions throughout the world varies from less than about 800 ppm of detergent composition (“low detergent concentration geographies”), for example about 667 ppm in Japan, to between about 800 ppm to about 2000 ppm (“medium detergent concentration geographies”), for example about 975 ppm in U.S. and about 1500 ppm in Brazil, to greater than about 2000 ppm (“high detergent concentration geographies”), for example about 4500 ppm to about 5000 ppm in Europe and about 6000 ppm in high suds phosphate builder geographies.

The concentrations of the typical wash solutions are determined empirically. For example, in the U.S., a typical washing machine holds a volume of about 64.4 L of wash solution. Accordingly, in order to obtain a concentration of about 975 ppm of detergent within the wash solution about 62.79 g of detergent composition must be added to the 64.4 L of wash solution. This amount is the typical amount measured into the wash water by the consumer using the measuring cup provided with the detergent.

As a further example, different geographies use different wash temperatures. The temperature of the wash water in Japan is typically less than that used in Europe. For example, the temperature of the wash water in North America and Japan is typically between about 10 and about 30° C. (e.g., about 20° C.), whereas the temperature of wash water in Europe is typically between about 30 and about 60° C. (e.g., about 40° C.). Accordingly, in certain embodiments, the detergent compositions described herein may be utilized at temperature from about 10° C. to about 60° C., or from about 20° C. to about 60° C., or from about 30° C. to about 60° C., or from about 40° C. to about 60° C., as well as all other combinations within the range of about 40° C. to about 55° C., and all ranges within 10° C. to 60° C. However, in the interest of saving energy, many consumers are switching to using cold water washing. In addition, in some further regions, cold water is typically used for laundry, as well as dish washing applications. In some embodiments, the “cold water washing” of the present disclosure utilizes washing at temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm # divided by 17.1 equals grains per gallon) of hardness minerals.

TABLE II
Water Hardness Levels

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

Accordingly, in some embodiments, the present disclosure provides Bsp Man4 polypeptides that show surprising wash performance in at least one set of wash conditions (e.g., water temperature, water hardness, and/or detergent concentration). In some embodiments, the Bsp Man4 polypeptides are comparable in wash performance to other endo-β-mannanases. In some embodiments, the Bsp Man4 polypeptides exhibit enhanced wash performance as compared to endo-β-mannanases currently commercially available. Thus, in some preferred embodiments, the Bsp Man4 polypeptides provided herein exhibit enhanced oxidative stability, enhanced thermal stability, enhanced cleaning capabilities under various conditions, and/or enhanced chelator stability. In addition, the Bsp Man4 polypeptides may find use in cleaning compositions that do not include detergents, again either alone or in combination with builders and stabilizers.

In some embodiments of the present disclosure, the cleaning compositions comprise at least one Bsp Man4 polypeptide of the present disclosure at a level from about 0.00001% to about 10% by weight of the composition and the balance (e.g., about 99.999% to about 90.0%) comprising cleaning adjunct materials by weight of composition. In other aspects of the present disclosure, the cleaning compositions comprises at least one Bsp Man4 polypeptide at a level of about 0.0001% to about 10%, about 0.001% to about 5%, about 0.001% to about 2%, about 0.005% to about 0.5% by weight of the composition and the balance of the cleaning composition (e.g., about 99.9999% to about 90.0%, about 99.999% to about 98%, about 99.995% to about 99.5% by weight) comprising cleaning adjunct materials.

In addition to the Bsp Man4 polypeptides provided herein, any other suitable endo-β-mannanases find use in the compositions of the present disclosure. Suitable endo-β-mannanases include, but are not limited to, endo-β-mannanases of the GH26 family of glycosyl hydrolases, endo-β-mannanases of the GH5 family of glycosyl hydrolases, acidic endo-β-mannanases, neutral endo-β-mannanases, and alkaline endo-β-mannanases. Examples of alkaline endo-β-mannanases include those described in U.S. Pat. Nos. 6,060,299, 6,566,114, and 6,602,842; WO 9535362A1, WO 9964573A1, and WO9964619A1. Additionally, suitable endo-β-mannanases include, but are not limited to those of animal, plant, fungal, or bacterial origin. Chemically or genetically modified mutants are encompassed by the present disclosure.

Examples of useful endo-β-mannanases include Bacillus endo-β-mannanases such as B. subtilis endo-β-mannanase (See, e.g., U.S. Pat. No. 6,060,299, and WO 9964573A1), B. sp. 1633 endo-β-mannanase (See, e.g., U.S. Pat. No. 6,566,114 and WO9964619A1), Bacillus sp. AAI12 endo-β-mannanase (See, e.g., U.S. Pat. No. 6,566,114 and WO9964619A1), B. sp. AA349 endo-β-mannanase (See, e.g., U.S. Pat. No. 6,566,114 and WO9964619A1), B. agaradhaerens NCIMB 40482 endo-β-mannanase (See, e.g., U.S. Pat. No. 6,566,114 and WO9964619A1), B. halodurans endo-β-mannanase, B. clausii endo-β-mannanase (See, e.g., U.S. Pat. No. 6,566,114 and WO9964619A1), B. licheniformis endo-β-mannanase (See, e.g., U.S. Pat. No. 6,566,114 and WO9964619A1), Humicola endo-β-mannanases such as H. insolens endo-β-mannanase (See, e.g., U.S. Pat. No. 6,566,114 and WO9964619A1), and Caldocellulosiruptor endo-β-mannanases such as C. sp. endo-β-mannanase (See, e.g., U.S. Pat. No. 6,566,114 and WO9964619A1).

Furthermore, a number of identified mannanases (i.e., endo-β-mannanases and exo-β-mannanases) find use in some embodiments of the present disclosure, including but not limited to Agaricus bisporus mannanase (See, Tang et al., [2001] Appl. Environ. Microbiol. 67: 2298-2303), Aspergillu tamarii mannanase (See, Civas et al., [1984] Biochem. J. 219: 857-863), Aspergillus aculeatus mannanase (See, Christgau et al., [1994] Biochem. Mol. Biol. Int. 33: 917-925), Aspergillus awamori mannanase (See, Setati et al., [2001] Protein Express Purif. 21: 105-114), Aspergillus fumigatus mannanase (See, Puchart et al., [2004] Biochimica et biophysica Acta. 1674: 239-250), Aspergillus niger mannanase (See, Ademark et al., [1998] J. Biotechnol. 63: 199-210), Aspergillus oryzae NRRL mannanase (See, Regalado et al., [2000] J. Sci. Food Agric. 80: 1343-1350), Aspergillus sulphureus mannanase (See, Chen et al., [2007] J. Biotechnol. 128(3): 452-461), Aspergillus terrus mannanase (See, Huang et al., [2007] Wei Sheng Wu Xue Bao. 47(2): 280-284), Bacillus agaradhaerens mannanase (See, U.S. Pat. No. 6,376,445.), Bacillus AM001 mannanase (See, Akino et al., [1989] Arch. Microbiol. 152: 10-15), Bacillus brevis mannanase (See, Araujo and Ward, [1990] J. Appl. Bacteriol. 68: 253-261), Bacillus circulans K-1 mannanase (See, Yoshida et al., [1998] Biosci. Biotechnol. Biochem. 62(3): 514-520), Bacillus polymyxa mannanase (See, Araujo and Ward, [1990] J. Appl. Bacteriol. 68: 253-261), Bacillus sp JAMB-750 mannanase (See, Hatada et al., [2005] Extremophiles. 9: 497-500), Bacillus sp. M50 mannanase (See, Chen et al., [2000] Wei Sheng Wu Xue Bao. 40: 62-68), Bacillus sp. N 16-5 mannanase (See, Yanhe et al., [2004] Extremophiles 8: 447-454), Bacillus stearothermophilu mannanase (See, Talbot and Sygusch, [1990] Appl. Environ. Microbiol. 56: 3505-3510), Bacillus subtilis mannanase (See, Mendoza et al., [1994] World J. Microbiol. Biotechnol. 10: 51-54), Bacillus subtilis B36 mannanase (Li et al., [2006] Z. Naturforsch (C). 61: 840-846), Bacillus subtilis BM9602 mannanase (See, Cui et al., [1999] Wei Sheng Wu Xue Bao. 39(1): 60-63), Bacillus subtilis SA-22 mannanase (See, Sun et al., [2003] Sheng Wu Gong Cheng Xue Bao. 19(3): 327-330), Bacillus subtilis 168 mannanase (See, Helow and Khattab, [1996] Acta Microbiol. Immunol. Hung. 43: 289-299), Bacteroides ovatus mannanase (See, Gherardini et al., [1987] J. Bacteriol. 169: 2038-2043), Bacteroides ruminicola mannanase (See, Matsushita et al., [1991] J. Bacteriol. 173: 6919-6926), Caldibacillus cellulovorans mannanase (See, Sunna et al., [2000] Appl. Environ. Microbiol. 66: 664-670), Caldocellulosiruptor saccharolyticus mannanase (See, Morris et al., [1995]Appl. Environ. Microbiol. 61: 2262-2269), Caldocellum saccharolyticum mannanase (See, Bicho et al., [1991] Appl. Microbiol. Biotechnol. 36: 337-343), Cellulomonas fimi mannanase (See, Stoll et al., [1999] Appl. Environ. Microbiol. 65(6):2598-2605), Clostridium butyricum/beijerinckii mannanase (See, Nakajima and Matsuura, [1997] Biosci. Biotechnol. Biochem. 61: 1739-1742), Clostridium cellulolyticum mannanase (See, Perret et al., [2004] Biotechnol. Appl. Biochem. 40: 255-259), Clostridium tertium mannanase (See, Kataoka and Tokiwa, [1998] J. Appl. Microbiol. 84: 357-367), Clostridium thermocellum mannanase (See, Halstead et al., [1999] Microbiol. 145: 3101-3108), Dictyoglomus thermophilum mannanase (See, Gibbs et al., [1999] Curr. Microbiol. 39(6): 351-357), Flavobacterium sp mannanase (See, Zakaria et al., [1998] Biosci. Biotechnol. Biochem. 62: 655-660), Gastropoda pulmonata mannanase (See, Charrier and Rouland, [2001] J. Expt. Zool. 290: 125-135), Littorina brevicula mannanase (See, Yamamura et al., [1996] Biosci. Biotechnol. Biochem. 60: 674-676), Lycopersicon esculentum mannanase (See, Filichkin et al., [2000] Plant Physiol. 134:1080-1087), Paenibacillus curdlanolyticus mannanase (See, Pason and Ratanakhanokchai, [2006] Appl. Environ. Microbiol. 72: 2483-2490), Paenibacillus polymyxa mannanase (See, Han et al., [2006] Appl. Microbiol. Biotechnol. 73(3): 618-630), Phanerochaete chrysosporium mannanase (See, Wymelenberg et al., [2005] J. Biotechnol. 118: 17-34), Piromyces sp. mannanase (See, Fanutti et al., [1995] J. Biol. Chem. 270(49): 29314-29322), Pomacea insulars mannanase (See, Yamamura et al., [1993] Biosci. Biotechnol. Biochem. 7: 1316-1319), Pseudomonas fluorescens sub sp. Cellulose mannanase (See, Braithwaite et al., [1995] Biochem J. 305: 1005-1010), Rhodothermus marinus mannanase (See, Politz et al., [2000] Appl. Microbiol. Biotechnol. 53 (6): 715-721), Sclerotium rolfsii mannanase (See, Sachslehner et al., [2000] J. Biotechnol. 80:127-134), Streptomyces galbus mannanase (See, Kansoh and Nagieb, [2004] Anton. van. Leeuwonhoek. 85: 103-114), Streptomyces lividans mannanase (See, Arcand et al., [1993] J. Biochem. 290: 857-863), Thermoanaerobacterium Polysaccharolyticum mannanase (See, Cann et al., [1999] J. Bacteriol. 181: 1643-1651), Thermomonospora fusca mannanase (See, Hilge et al., [1998] Structure 6: 1433-1444), Thermotoga maritima mannanase (See, Parker et al., [2001] Biotechnol. Bioeng. 75(3): 322-333), Thermotoga neapolitana mannanase (See, Duffaud et al., [1997] Appl. Environ. Microbiol. 63: 169-177), Trichoderma harzanium strain T4 mannanase (See, Franco et al., [2004] Biotechnol Appl. Biochem. 40: 255-259), Trichoderma reesei mannanase (See, Stalbrand et al., [1993] J. Biotechnol. 29: 229-242), and Vibrio sp. mannanase (See, Tamaru et al., [1997] J. Ferment. Bioeng. 83: 201-205).

Additional suitable endo-β-mannanases include commercially available endo-β-mannanases such as HEMICELL® (Chemgen); GAMANASE® and MANNAWAY®, (Novozymes A/S, Denmark); PURABRITE™ and MANNASTAR™ (Genencor, A Danisco Division, Palo Alto, Calif.); and PYROLASE® 160 and PYROLASE® 200 (Diversa).

In some embodiments of the present disclosure, the cleaning compositions of the present disclosure further comprise endo-β-mannanases at a level from about 0.00001% to about 10% of additional endo-β-mannanase by weight of the composition and the balance of cleaning adjunct materials by weight of composition. In other aspects of the present disclosure, the cleaning compositions of the present disclosure also comprise endo-β-mannanases at a level of about 0.0001% to about 10%, about 0.001% to about 5%, about 0.001% to about 2%, about 0.005% to about 0.5% endo-β-mannanase by weight of the composition.

In some embodiments of the present disclosure, any suitable protease may be used. Suitable proteases include those of animal, vegetable or microbial origin. In some embodiments, chemically or genetically modified mutants are included. In some embodiments, the protease is a serine protease, preferably an alkaline microbial protease or a trypsin-like protease. Various proteases are described in PCT applications WO 95/23221 and WO 92/21760; U.S. Pat. Publication No. 2008/0090747; and U.S. Pat. Nos. 5,801,039; 5,340,735; 5,500,364; 5,855,625; U.S. RE 34,606; U.S. Pat. Nos. 5,955,340; 5,700,676; 6,312,936; 6,482,628; and various other patents. In some further embodiments, metalloproteases find use in the present disclosure, including but not limited to the neutral metalloprotease described in PCT application WO 07/044,993. Commercially available proteases that find use in the present disclosure include, but are not limited to PURAFECT®, PURAFECT® PRIME, and PROPERASE® (Genencor, A Danisco Division, Palo Alto, Calif.). Additionally, commercially available proteases that find use in the present disclosure include, but are not limited to ALCALASE®, EVERLASE®, LIQUINASE®, POLARZYME®, OVOZYME® and SAVINASE® (Novozymes A/S, Denmark).

In some embodiments of the present disclosure, any suitable amylase may be used. In some embodiments, any amylase (e.g., alpha and/or beta) suitable for use in alkaline solutions also find use. Suitable amylases include, but are not limited to those of bacterial or fungal origin. Chemically or genetically modified mutants are included in some embodiments. Amylases that find use in the present disclosure include, but are not limited to α-amylases obtained from B. licheniformis (See, e.g., GB 1,296,839). Commercially available amylases that find use in the present disclosure include, but are not limited to DURAMYL®, TERMAMYL®, FUNGAMYL®, STAINZYME®, STAINZYME PLUS®, STAINZYME ULTRA®, and BAN™ (Novozymes A/S, Denmark), as well as PURASTAR®, POWERASE™, RAPIDASE®, and MAXAMYL® P (Genencor, A Danisco Division, Palo Alto, Calif.).

In some embodiments of the present disclosure, the disclosed cleaning compositions further comprise amylases at a level from about 0.00001% to about 10% of additional amylase by weight of the composition and the balance of cleaning adjunct materials by weight of composition. In other aspects of the present disclosure, the cleaning compositions also comprise amylases at a level of about 0.0001% to about 10%, about 0.001% to about 5%, about 0.001% to about 2%, about 0.005% to about 0.5% amylase by weight of the composition.

In some embodiments of the present disclosure, any suitable pectin degrading enzyme may be used. As used herein, “pectin degrading enzyme(s)” encompass arabinanase (EC 3.2.1.99), galactanases (EC 3.2.1.89), polygalacturonase (EC 3.2.1.15) exo-polygalacturonase (EC 3.2.1.67), exo-poly-alpha-galacturonidase (EC 3.2.1.82), pectin lyase (EC 4.2.2.10), pectin esterase (EC 3.2.1.11), pectate lyase (EC 4.2.2.2), exo-polygalacturonate lyase (EC 4.2.2.9) and hemicellulases such as endo-1,3-β-xylosidase (EC 3.2.1.32), xylan-1,4-β-xylosidase (EC 3.2.1.37) and α-L-arabinofuranosidase (EC 3.2.1.55). Pectin degrading enzymes are natural mixtures of the above mentioned enzymatic activities. Pectin enzymes therefore include the pectin methylesterases which hdyrolyse the pectin methyl ester linkages, polygalacturonases which cleave the glycosidic bonds between galacturonic acid molecules, and the pectin transeliminases or lyases which act on the pectic acids to bring about non-hydrolytic cleavage of α-1,4 glycosidic linkages to form unsaturated derivatives of galacturonic acid.

Suitable pectin degrading enzymes include those of plant, fungal, or microbial origin. In some embodiments, chemically or genetically modified mutants are included. In some embodiments, the pectin degrading enzymes are alkaline pectin degrading enzymes, i.e., enzymes having an enzymatic activity of at least 10%, preferably at least 25%, more preferably at least 40% of their maximum activity at a pH of from about 7.0 to about 12. In certain other embodiments, the pectin degrading enzymes are enzymes having their maximum activity at a pH of from about 7.0 to about 12. Alkaline pectin degrading enzymes are produced by alkalophilic microorganisms e.g., bacterial, fungal, and yeast microorganisms such as Bacillus species. In some embodiments, the microorganisms are Bacillus firmus, Bacillus circulans, and Bacillus subtilis as described in JP 56131376 and JP 56068393. Alkaline pectin decomposing enzymes may include but are not limited to galacturn-1,4-α-galacturonase (EC 3.2.1.67), poly-galacturonase activities (EC 3.2.1.15, pectin esterase (EC 3.1.1.11), pectate lyase (EC 4.2.2.2) and their iso enzymes. Alkaline pectin decomposing enzymes can be produced by the Erwinia species. In some embodiments, the alkaline pectin decomposing enzymes are produced by E. chrysanthemi, E. carotovora, E. amylovora, E. herbicola, and E. dissolvens as described in JP 59066588, JP 63042988, and in World J. Microbiol. Microbiotechnol. (8, 2, 115-120) 1992. In certain other embodiments, the alkaline pectin enzymes are produced by Bacillus species as disclosed in JP 73006557 and Agr. Biol. Chem. (1972), 36 (2) 285-93.

In some embodiments of the present disclosure, the disclosed cleaning compositions further comprise pectin degrading enzymes at a level from about 0.00001% to about 10% of additional pectin degrading enzyme by weight of the composition and the balance of cleaning adjunct materials by weight of composition. In other aspects of the present disclosure, the cleaning compositions also comprise pectin degrading enzymes at a level of about 0.0001% to about 10%, about 0.001% to about 5%, about 0.001% to about 2%, about 0.005% to about 0.5% pectin degrading enzyme by weight of the composition.

In some other embodiments, any suitable xyloglucanase finds used in the cleaning compositions of the present disclosure. Suitable xyloglucanases include, but are not limited to those of plant, fungal, or bacterial origin. Chemically or genetically modified mutants are included in some embodiments. As used herein, “xyloglucanase(s)” encompass the family of enzymes described by Vincken and Voragen at Wageningen University [Vincken et al (1994) Plant Physiol., 104, 99-107] and are able to degrade xyloglucans as described in Hayashi et al (1989) Plant. Physiol. Plant Mol. Biol., 40, 139-168. Vincken et al demonstrated the removal of xyloglucan coating from cellulose of the isolated apple cell wall by a xyloglucanase purified from Trichoderma viride (endo-IV-glucanase). This enzyme enhances the enzymatic degradation of cell wall-embedded cellulose and work in synergy with pectic enzymes. Rapidase LIQ+ from Gist-Brocades contains a xyloglucanase activity.

In some embodiments of the present disclosure, the disclosed cleaning compositions further comprise xyloglucanases at a level from about 0.00001% to about 10% of additional xyloglucanase by weight of the composition and the balance of cleaning adjunct materials by weight of composition. In other aspects of the present disclosure, the cleaning compositions also comprise xyloglucanases at a level of about 0.0001% to about 10%, about 0.001% to about 5%, about 0.001% to about 2%, about 0.005% to about 0.5% xyloglucanase by weight of the composition. In certain other embodiments, xyloglucanases for specific applications are alkaline xyloglucanases, i.e., enzymes having an enzymatic activity of at least 10%, preferably at lest 25%, more preferably at least 40% of their maximum activity at a pH ranging from 7 to 12. In certain other embodiments, the xyloglucanases are enzymes having their maximum activity at a pH of from about 7.0 to about 12.

In some further embodiments, any suitable cellulase finds used in the cleaning compositions of the present disclosure. Suitable cellulases include, but are not limited to those of bacterial or fungal origin. Chemically or genetically modified mutants are included in some embodiments. Suitable cellulases include, but are not limited to Humicola insolens cellulases (See, e.g., U.S. Pat. No. 4,435,307). Especially suitable cellulases are the cellulases having color care benefits (See, e.g., EP 0 495 257). Commercially available cellulases that find use in the present disclosure include, but are not limited to ENDOLASE®, CELLUCLEAN®, CELLUZYME®, CAREZYME® (Novozymes A/S, Denmark). Additional commercially available cellulases include PURADEX® (Genencor, A Danisco Division, Palo Alto, Calif.) and KAC-500(B)™ (Kao Corporation). In some embodiments, cellulases are incorporated as portions or fragments of mature wild-type or variant cellulases, wherein a portion of the N-terminus is deleted (See, e.g., U.S. Pat. No. 5,874,276). In some embodiments, the cleaning compositions of the present disclosure further comprise cellulases at a level from about 0.00001% to about 10% of additional cellulase by weight of the composition and the balance of cleaning adjunct materials by weight of composition. In other aspects of the present disclosure, the cleaning compositions also comprise cellulases at a level of about 0.0001% to about 10%, about 0.001% to about 5%, about 0.001% to about 2%, about 0.005% to about 0.5% cellulase by weight of the composition.

In still further embodiments, any lipase suitable for use in detergent compositions also finds use in the present disclosure. Suitable lipases include, but are not limited to those of bacterial or fungal origin. Chemically or genetically modified mutants are included in some embodiments. Examples of useful lipases include Humicola lanuginosa lipase (See, e.g., EP 258 068, and EP 305 216), Rhizomucor miehei lipase (See, e.g., EP 238 023), Candida lipase, such as C. antarctica lipase (e.g., the C. antarctica lipase A or B; see, e.g., EP 214 761), Pseudomonas lipases such as P. alcaligenes lipase and P. pseudoalcaligenes lipase (See, e.g., EP 218 272), P. cepacia lipase (See, e.g., EP 331 376), P. stutzeri lipase (See, e.g., GB 1,372,034), P. fluorescens lipase, Bacillus lipase (e.g., B. subtilis lipase [Dartois et al., (1993) Biochem. Biophys. Acta 1131:253-260]; B. stearothermophilus lipase [See, e.g., JP 64/744992]; and B. pumilus lipase [See, e.g., WO 91/16422]). Furthermore, a number of cloned lipases find use in some embodiments of the present disclosure, including but not limited to Penicillium camembertii lipase (See, Yamaguchi et al., [1991] Gene 103:61-67), Geotricum candidum lipase (See, Schimada et al., [1989] J. Biochem. 106:383-388), and various Rhizopus lipases such as R. delemar lipase (See, Hass et al., [1991] Gene 109:117-113), R. niveus lipase (Kugimiya et al., [1992] Biosci. Biotech. Biochem. 56:716-719), and R. oryzae lipase. Other types of lipolytic enzymes such as cutinases also find use in some embodiments of the present disclosure, including but not limited to the cutinase derived from Pseudomonas mendocina (See, WO 88/09367), and the cutinase derived from Fusarium solani pisi (See, WO 90/09446). Additional suitable lipases include commercially available lipases such as M1 LIPASE™, LUMA FAST™, and LIPOMAX™ (Genencor, A Danisco Division, Palo Alto, Calif.); LIPEX®, LIPOCLEAN®, LIPOLASE® and LIPOLASE® ULTRA (Novozymes A/S, Denmark); and LIPASE P™“Amano” (Amano Pharmaceutical Co. Ltd., Japan).

In some embodiments, the disclosed cleaning compositions further comprise lipases at a level from about 0.00001% to about 10% of additional lipase by weight of the composition and the balance of cleaning adjunct materials by weight of composition. In other aspects of the present disclosure, the cleaning compositions also comprise lipases at a level of about 0.0001% to about 10%, about 0.001% to about 5%, about 0.001% to about 2%, about 0.005% to about 0.5% lipase by weight of the composition.

In some embodiments, peroxidases are used in combination with hydrogen peroxide or a source thereof (e.g., a percarbonate, perborate or persulfate) in the compositions of the present disclosure. In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (See, e.g., WO 94/12621 and WO 95/01426). Suitable peroxidases/oxidases include, but are not limited to those of plant, bacterial or fungal origin. Chemically or genetically modified mutants are included in some embodiments. In some embodiments, the cleaning compositions of the present disclosure further comprise peroxidase and/or oxidase enzymes at a level from about 0.00001% to about 10% of additional peroxidase and/or oxidase by weight of the composition and the balance of cleaning adjunct materials by weight of composition. In other aspects of the present disclosure, the cleaning compositions also comprise peroxidase and/or oxidase enzymes at a level of about 0.0001% to about 10%, about 0.001% to about 5%, about 0.001% to about 2%, about 0.005% to about 0.5% peroxidase and/or oxidase enzymes by weight of the composition.

In some embodiments, additional enzymes find use, including but not limited to perhydrolases (See, e.g., WO 05/056782). In addition, in some particularly preferred embodiments, mixtures of the above mentioned enzymes are encompassed herein, in particular one or more additional protease, amylase, lipase, mannanase, and/or at least one cellulase. Indeed, it is contemplated that various mixtures of these enzymes will find use in the present disclosure. It is also contemplated that the varying levels of the Bsp Man4 polypeptide(s) and one or more additional enzymes may both independently range to about 10%, the balance of the cleaning composition being cleaning adjunct materials. The specific selection of cleaning adjunct materials are readily made by considering the surface, item, or fabric to be cleaned, and the desired form of the composition for the cleaning conditions during use (e.g., through the wash detergent use).

Examples of suitable cleaning adjunct materials include, but are not limited to, surfactants, builders, bleaches, bleach activators, bleach catalysts, other enzymes, enzyme stabilizing systems, chelants, optical brighteners, soil release polymers, dye transfer agents, dye transfer inhibiting agents, catalytic materials, hydrogen peroxide, sources of hydrogen peroxide, preformed peracids, polymeric dispersing agents, clay soil removal agents, structure elasticizing agents, dispersants, suds suppressors, dyes, perfumes, colorants, filler salts, hydrotropes, photoactivators, fluorescers, fabric conditioners, fabric softeners, carriers, hydrotropes, processing aids, solvents, pigments, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See, e.g., U.S. Pat. Nos. 6,610,642; 6,605,458; 5,705,464; 5,710,115; 5,698,504; 5,695,679; 5,686,014; and 5,646,101; all of which are incorporated herein by reference). Embodiments of specific cleaning composition materials are exemplified in detail below. In embodiments in which the cleaning adjunct materials are not compatible with the disclosed Bsp Man4 polypeptides in the cleaning compositions, then suitable methods of keeping the cleaning adjunct materials and the endo-β-mannanase(s) separated (i.e., not in contact with each other) until combination of the two components is appropriate are used. Such separation methods include any suitable method known in the art (e.g., gelcaps, encapsulation, tablets, physical separation, etc.).

In some preferred embodiments, an effective amount of one or more Bsp Man4 polypeptide(s) provided herein are included in compositions useful for cleaning a variety of surfaces in need of stain removal. Such cleaning compositions include cleaning compositions for such applications as cleaning hard surfaces, fabrics, and dishes. Indeed, in some embodiments, the present disclosure provides fabric cleaning compositions, while in other embodiments, the present disclosure provides non-fabric cleaning compositions. Notably, the present disclosure also provides cleaning compositions suitable for personal care, including oral care (including dentrifices, toothpastes, mouthwashes, etc., as well as denture cleaning compositions), skin, and hair cleaning compositions. Additionally, in still other embodiments, the present disclosure provides fabric softening compositions. It is intended that the present disclosure encompass detergent compositions in any form (i.e., liquid, granular, bar, semi-solid, gels, emulsions, tablets, capsules, etc.).

By way of example, several cleaning compositions wherein the disclosed Bsp Man4 polypeptides find use are described in greater detail below. In some embodiments in which the disclosed cleaning compositions are formulated as compositions suitable for use in laundry machine washing method(s), the compositions of the present disclosure preferably contain at least one surfactant and at least one builder compound, as well as one or more cleaning adjunct materials preferably selected from organic polymeric compounds, bleaching agents, additional enzymes, suds suppressors, dispersants, lime-soap dispersants, soil suspension and anti-redeposition agents and corrosion inhibitors. In some embodiments, laundry compositions also contain softening agents (i.e., as additional cleaning adjunct materials). The compositions of the present disclosure also find use detergent additive products in solid or liquid form. Such additive products are intended to supplement and/or boost the performance of conventional detergent compositions and can be added at any stage of the cleaning process. In some embodiments, the density of the laundry detergent compositions herein ranges from about 400 to about 1200 g/liter, while in other embodiments, it ranges from about 500 to about 950 g/liter of composition measured at 20° C.

In embodiments formulated as compositions for use in manual dishwashing methods, the compositions of the disclosure preferably contain at least one surfactant and preferably at least one additional cleaning adjunct material selected from organic polymeric compounds, suds enhancing agents, group II metal ions, solvents, hydrotropes, and additional enzymes.

In some embodiments, various cleaning compositions such as those provided in U.S. Pat. No. 6,605,458 find use with the Bsp Man4 polypeptides of the present disclosure. Thus, in some embodiments, the compositions comprising at least one Bsp Man4 polypeptide of the present disclosure is a compact granular fabric cleaning composition, while in other embodiments, the composition is a granular fabric cleaning composition useful in the laundering of colored fabrics, in further embodiments, the composition is a granular fabric cleaning composition which provides softening through the wash capacity, in additional embodiments, the composition is a heavy duty liquid fabric cleaning composition. In some embodiments, the compositions comprising at least one Bsp Man4 polypeptide of the present disclosure are fabric cleaning compositions such as those described in U.S. Pat. Nos. 6,610,642 and 6,376,450. In addition, the Bsp Man4 polypeptides of the present disclosure find use in granular laundry detergent compositions of particular utility under European or Japanese washing conditions (See, e.g., U.S. Pat. No. 6,610,642).

In some alternative embodiments, the present disclosure provides hard surface cleaning compositions comprising at least one Bsp Man4 polypeptide provided herein. Thus, in some embodiments, the compositions comprising at least one Bsp Man4 polypeptide of the present disclosure is a hard surface cleaning composition such as those described in U.S. Pat. Nos. 6,610,642; 6,376,450; and 6,376,450.

In yet further embodiments, the present disclosure provides dishwashing compositions comprising at least one Bsp Man4 polypeptide provided herein. Thus, in some embodiments, the compositions comprising at least one Bsp Man4 polypeptide of the present disclosure is a hard surface cleaning composition such as those in U.S. Pat. Nos. 6,610,642 and 6,376,450. In some still further embodiments, the present disclosure provides dishwashing compositions comprising at least one Bsp Man4 polypeptide provided herein. In some further embodiments, the compositions comprising at least one Bsp Man4 polypeptide of the present disclosure comprise oral care compositions such as those in U.S. Pat. Nos. 6,376,450 and 6,605,458. The formulations and descriptions of the compounds and cleaning adjunct materials contained in the aforementioned U.S. Pat. Nos. 6,376,450; 6,605,458; and 6,610,642 find use with the Bsp Man4 polypeptides provided herein.

In still further embodiments, the compositions comprising at least one Bsp Man4 polypeptide of the present disclosure comprise fabric softening compositions such as those in GB-A1 400898, GB-A1 514 276, EP 0 011 340, EP 0 026 528, EP 0 242 919, EP 0 299 575, EP 0 313 146, and U.S. Pat. No. 5,019,292. The formulations and descriptions of the compounds and softening agents contained in the aforementioned GB-A1 400898, GB-A1 514 276, EP 0 011 340, EP 0 026 528, EP 0 242 919, EP 0 299 575, EP 0 313 146, and U.S. Pat. No. 5,019,292 find use with the Bsp Man4 polypeptides provided herein

The cleaning compositions of the present disclosure are formulated into any suitable form and prepared by any process chosen by the formulator, non-limiting examples of which are described in U.S. Pat. Nos. 5,879,584; 5,691,297; 5,574,005; 5,569,645; 5,565,422; 5,516,448; 5,489,392; and 5,486,303; all of which are incorporated herein by reference. When a low pH cleaning composition is desired, the pH of such composition is adjusted via the addition of a material such as monoethanolamine or an acidic material such as HCl.

While not essential for the purposes of the present disclosure, the non-limiting list of adjuncts illustrated hereinafter are suitable for use in the instant cleaning compositions. In some embodiments, these adjuncts are incorporated for example, to assist or enhance cleaning performance, for treatment of the substrate to be cleaned, or to modify the aesthetics of the cleaning composition as is the case with perfumes, colorants, dyes or the like. It is understood that such adjuncts are in addition to the Bsp Man4 polypeptides of the present disclosure. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282; 6,306,812; and 6,326,348 are incorporated by reference. The aforementioned adjunct ingredients may constitute the balance of the cleaning compositions of the present disclosure.

In some embodiments, the cleaning compositions according to the present disclosure comprise at least one surfactant and/or a surfactant system wherein the surfactant is selected from nonionic surfactants, anionic surfactants, cationic surfactants, ampholytic surfactants, zwitterionic surfactants, semi-polar nonionic surfactants, and mixtures thereof. In some low pH cleaning composition embodiments (e.g., compositions having a neat pH of from about 3 to about 5), the composition typically does not contain alkyl ethoxylated sulfate, as it is believed that such surfactant may be hydrolyzed by such compositions' acidic contents. In some embodiments, the surfactant is present at a level of from about 0.1% to about 60%, while in alternative embodiments the level is from about 1% to about 50%, while in still further embodiments the level is from about 5% to about 40%, by weight of the cleaning composition.

In some embodiments, the cleaning compositions of the present disclosure contain at least one chelating agent. Suitable chelating agents may include, but are not limited to copper, iron, and/or manganese chelating agents, and mixtures thereof. In embodiments in which at least one chelating agent is used, the cleaning compositions of the present disclosure comprise from about 0.1% to about 15% or even from about 3.0% to about 10% chelating agent by weight of the subject cleaning composition.

In some still further embodiments, the cleaning compositions provided herein contain at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

As indicated herein, in some embodiments, anti-redeposition agents find use in some embodiments of the present disclosure. In some preferred embodiments, non-ionic surfactants find use. For example, in automatic dishwashing embodiments, non-ionic surfactants find use for surface modification purposes, in particular for sheeting, to avoid filming and spotting and to improve shine. These non-ionic surfactants also find use in preventing the re-deposition of soils. In some preferred embodiments, the anti-redeposition agent is a non-ionic surfactant as known in the art (See, e.g., EP 2 100 949).

In some embodiments, the cleaning compositions of the present disclosure include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, and polyvinylimidazoles, or mixtures thereof. In embodiments in which at least one dye transfer inhibiting agent is used, the cleaning compositions of the present disclosure comprise from about 0.0001% to about 10%, from about 0.01% to about 5%, or even from about 0.1% to about 3% by weight of the cleaning composition.

In some embodiments, silicates are included within the compositions of the present disclosure. In some such embodiments, sodium silicates (e.g., sodium disilicate, sodium metasilicate, and crystalline phyllosilicates) find use. In some embodiments, silicates are present at a level of from about 1% to about 20%. In some preferred embodiments, silicates are present at a level of from about 5% to about 15% by weight of the composition.

In some still additional embodiments, the cleaning compositions of the present disclosure also contain dispersants. Suitable water-soluble organic materials include, but are not limited to the homo- or co-polymeric acids or their salts, in which the polycarboxylic acid comprises at least two carboxyl radicals separated from each other by not more than two carbon atoms.

In some further embodiments, the enzymes used in the cleaning compositions are stabilized by any suitable technique. In some embodiments, the enzymes employed herein are stabilized by the presence of water-soluble sources of calcium and/or magnesium ions in the finished compositions that provide such ions to the enzymes. In some embodiments, the enzyme stabilizers include oligosaccharides, polysaccharides, and inorganic divalent metal salts, including alkaline earth metals, such as calcium salts. It is contemplated that various techniques for enzyme stabilization will find use in the present disclosure. For example, in some embodiments, the enzymes employed herein are stabilized by the presence of water-soluble sources of zinc (II), calcium (II), and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV). Chlorides and sulfates also find use in some embodiments of the present disclosure. Examples of suitable oligosaccharides and polysaccharides (e.g., dextrins) are known in the art (See, e.g., WO 07/145,964). In some embodiments, reversible protease inhibitors also find use, such as boron-containing compounds (e.g., borate, 4-formyl phenyl boronic acid) and/or a tripeptide aldehyde find use to further improve stability, as desired.

In some embodiments, bleaches, bleach activators, and/or bleach catalysts are present in the compositions of the present disclosure. In some embodiments, the cleaning compositions of the present disclosure comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Any suitable salt known in the art finds use in the present disclosure (See, e.g., EP 2 100 949).

In some embodiments, bleach activators are used in the compositions of the present disclosure. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxycarboxylic acids having preferably from about 1 to about 10 carbon atoms, in particular from about 2 to about 4 carbon atoms, and/or optionally substituted perbenzoic acid. Additional bleach activators are known in the art and find use in the present disclosure (See, e.g., EP 2 100 949).

In addition, in some embodiments and as further described herein, the cleaning compositions of the present disclosure further comprise at least one bleach catalyst. In some embodiments, the manganese triazacyclononane and related complexes find use, as well as cobalt, copper, manganese, and iron complexes. Additional bleach catalysts find use in the present disclosure (See, e.g., U.S. Pat. No. 4,246,612; U.S. Pat. No. 5,227,084; U.S. Pat. No. 4,810,410; WO 99/06521; and EP 2 100 949).

In some embodiments, the cleaning compositions of the present disclosure contain one or more catalytic metal complexes. In some embodiments, a metal-containing bleach catalyst finds use. In some preferred embodiments, the metal bleach catalyst comprises a catalyst system comprising a transition metal cation of defined bleach catalytic activity, (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See, e.g., U.S. Pat. No. 4,430,243). In some embodiments, the cleaning compositions of the present disclosure are catalyzed by means of a manganese compound. Such compounds and levels of use are well known in the art (See, e.g., U.S. Pat. No. 5,576,282). In additional embodiments, cobalt bleach catalysts find use in the cleaning compositions of the present disclosure. Various cobalt bleach catalysts are known in the art (See, e.g., U.S. Pat. Nos. 5,597,936 and 5,595,967) and are readily prepared by known procedures.

In some additional embodiments, the cleaning compositions of the present disclosure include a transition metal complex of a macropolycyclic rigid ligand (MRL). As a practical matter, and not by way of limitation, in some embodiments, the compositions and cleaning processes provided by the present disclosure are adjusted to provide on the order of at least one part per hundred million of the active MRL species in the aqueous washing medium, and in some preferred embodiments, provide from about 0.005 ppm to about 25 ppm, more preferably from about 0.05 ppm to about 10 ppm, and most preferably from about 0.1 ppm to about 5 ppm, of the MRL in the wash liquor.

In some embodiments, preferred transition-metals in the instant transition-metal bleach catalyst include, but are not limited to manganese, iron, and chromium. Preferred MRLs also include, but are not limited to special ultra-rigid ligands that are cross-bridged (e.g., 5,12-diethyl-1,5,8,12-tetraazabicyclo[6.6.2] hexadecane). Suitable transition metal MRLs are readily prepared by known procedures (See, e.g., WO 2000/32601 and U.S. Pat. No. 6,225,464).

In some embodiments, the cleaning compositions of the present disclosure comprise metal care agents. Metal care agents find use in preventing and/or reducing the tarnishing, corrosion, and/or oxidation of metals, including aluminum, stainless steel, and non-ferrous metals (e.g., silver and copper). Suitable metal care agents include those described in EP 2 100 949, WO 94/26860, and WO 94/26859). In some embodiments, the metal care agent is a zinc salt. In some further embodiments, the cleaning compositions of the present disclosure comprise from about 0.1% to about 5% by weight of one or more metal care agent.

As indicated above, the cleaning compositions of the present disclosure are formulated into any suitable form and prepared by any process chosen by the formulator, non-limiting examples of which are described in U.S. Pat. Nos. 5,879,584; 5,691,297; 5,574,005; 5,569,645; 5,516,448; 5,489,392; and 5,486,303; all of which are incorporated herein by reference. In some embodiments in which a low pH cleaning composition is desired, the pH of such composition is adjusted via the addition of an acidic material such as HCl.

The cleaning compositions disclosed herein of find use in cleaning a situs (e.g., a surface, dishware, or fabric). Typically, at least a portion of the situs is contacted with an embodiment of the present cleaning composition, in neat form or diluted in wash liquor, and then the situs is optionally washed and/or rinsed. For purposes of the present disclosure, “washing” includes but is not limited to, scrubbing and mechanical agitation. In some embodiments, the cleaning compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric mass ratio is typically from about 1:1 to about 30:1.

### VI. Bsp Man4 Polypeptides as Chemical Reagents

The preference of Bsp Man4 for polysaccharide chains containing mannose units, including but not limited to mannans, galactomannans, and glucomannans, makes the present polypeptides particularly useful for performing mannan hydrolysis reactions involving polysaccharide substrates containing 1, 4-β-D-mannosidic linkages.

In general terms, a donor molecule is incubated in the presence of an isolated Bsp Man4 polypeptide or fragment or variant thereof under conditions suitable for performing a mannan hydrolysis reaction, followed by, optionally, isolating a product from the reaction. Alternatively, in the context of a foodstuff, the product may become a component of the foodstuff without isolation. In certain embodiments, the donor molecule is a polysaccharide chain comprising mannose units, including but not limited to mannans, glucomannans, galactomannans, and galactoglucomannans.

### VII. Bsp Man4 Polypeptides for Food Processing and Animal Feed

Several anti-nutritional factors can limit the use of specific plant material in the preparation of animal feed and food for humans. For example, plant material containing oligomannans such as mannan, galactomannan, glucomannan and galactoglucomannan can reduce the digestibility and absorption of nutritional compounds such as minerals, vitamins, sugars and fats by the animals. The negative effects are in particular due to the high viscosity of the mannan-containing polymers and to the ability of the mannan-containing polymers to adsorb nutritional compounds. These effects are reduced through the use of mannan-containing polymers degrading enzymes, namely endo-β-mannanase enzymes such as the Bsp Man4 polypeptides described herein, which permit a higher proportion of mannan-containing polymers containing cheap plant material to be included in the feed resulting in a reduction of feed costs. Additionally, through the activity of the Bsp Man4 polypeptides, mannan-containing polymers are broken down to simpler sugars, which can be more readily assimilated to provide additional energy. Accordingly, compositions comprising any of the Bsp Man4 polypeptides described herein preferably used for processing and/or manufacturing of food or animal feed.

In one aspect of the invention, there is provided a bread improver composition comprising any of the Bsp Man4 polypeptides of the current invention, optionally with a source of mannan or glucomannan or galactomannan present, and further optionally with other enzymes present.

In general terms animal feed containing plant material is incubated in the presence of an isolated Bsp Man4 polypeptide or fragment or variant thereof under conditions suitable for breaking down mannan-containing polymers.

The Bsp Man4 polypeptides of the present disclosure are useful as additives to feed for non-human animals. The term non-human animal includes all non-ruminant and ruminant animals. In a particular embodiment, the non-ruminant animal, is selected from the group consisting of, but not limited to, horses and monogastric animals such as, but not limited to, pigs, poultry, swine and fish. In further embodiments, the pig may be, but not limited to, a piglet, a growing pig, and a sow; the poultry may be, but not limited to, a turkey, a duck and a chicken including, but not limited to, a broiler chick, a layer; and fish including but not limited to salmon, trout, tilapia, catfish and carps; and crustaceans including but not limited to shrimps and prawns.such as poultry and swine, In a further embodiment, the non-human animal is a ruminant animal including, but not limited to, cattle, young calves, goats, sheep, giraffes, bison, moose, elk, yaks, water buffalo, deer, camels, alpacas, llamas, antelope, pronghorn, and nilgai. The Bag Man1 polypeptides of the present disclosure are also useful as additives. The Bsp Man4 polypeptides of the present disclosure are also useful for human food. In some embodiments, the Bsp Man4 polypeptides are used to pretreat the feed instead of as a feed additive. In some preferred embodiment, the Bsp Man4 polypeptides are added to or used to pretreat feed for weanling pigs, nursery pigs, piglets, fattening pigs, growing pigs, finishing pigs, laying hens, broiler chicks, turkeys. In some embodiment, the Bsp Man4 polypeptides are added to or used to pretreat feed from plant material such as palm kernel, coconut, konjac, locust bean gum, gum guar, soy beans, barley, oats, flax, wheat, corn, linseed, citrus pulp, cottonseed, groundnut, rapeseed, sunflower, peas, and lupines.

Since the Bsp Man4 polypeptides of the present disclosure are thermostable enzymes, they find used in processes of producing pelleted feed in which heat is applied to the feed mixture before the pelleting step, as it is the case in most commercial pellet mills. The Bsp Man4 polypeptides are added to the other feed ingredients in advance of the pelleting step or after the pelleting step to the already formed feed pellets.

In compositions containing any of the disclosed Bsp Man4 polypeptides intended for food processing or as a feed supplement, the compositions optionally contain other substituents such as coloring agents, aroma compounds, stabilizers, vitamins, minerals, other feed or food enhancing enzymes and the like. This applies in particular to the so-called pre-mixes. Food additives according to this present invention may be combined with other food components to produce processed food products. The resulting, combined food additive is mixed in an appropriate amount with other food components such as cereal or plant proteins to form a processed food product.

Accordingly, the present invention relates to an animal feed composition and/or animal feed additive composition and/or pet food comprising the Bsp Man4 polypeptides.

The present invention further relates to a method for preparing such animal feed composition and/or animal feed additive composition and/or pet food comprising mixing the Bsp Man4 polypeptides with one or more animal feed ingredients and/or animal feed additive ingredients and/or pet food ingredients.

Furthermore, the present invention relates to the use of the Bsp Man4 polypeptides in the preparation of an animal feed composition and/or animal feed additive composition and/or pet food.

In the present context, it is intended that the term pet food is understood to mean a food for a household animal such as, but not limited to dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

The terms animal feed composition, feedstuff and fodder are used interchangeably and may comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS) (particularly corn based Distillers Dried Grain Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

### VIIIa. Bsp Man4 Polypeptides for Fermented Beverages, Such as Beer

In aspects of the invention the food composition or additive may be liquid or solid.

In an aspect of the invention the food composition is a beverage, including, but not limited to, a fermented beverage such as beer and wine, comprising any of the Bsp Man4 polypeptides of the invention.

In the context of the present invention, the term “fermented beverage” is meant to comprise any beverage produced by a method comprising a fermentation process, such as a microbial fermentation, such as a bacterial and/or yeast fermentation.

In an aspect of the invention the fermented beverage is beer. The term “beer” is meant to comprise any fermented wort produced by fermentation/brewing of a starch-containing plant material. Often, beer is produced from malt or adjunct, or any combination of malt and adjunct as the starch-containing plant material. As used herein the term “malt” is understood as any malted cereal grain, such as malted barley or wheat.

As used herein the term “adjunct” refers to any starch and/or sugar containing plant material which is not malt, such as barley or wheat malt. As examples of adjuncts, mention can be made of materials such as common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, cassaya and syrups, such as corn syrup, sugar cane syrup, inverted sugar syrup, barley and/or wheat syrups, and the like may be used as a source of starch

As used herein, the term “mash” refers to an aqueous slurry of any starch and/or sugar containing plant material such as grist, e.g. comprising crushed barley malt, crushed barley, and/or other adjunct or a combination hereof, mixed with water later to be separated into wort and spent grains.

As used herein, the term “wort” refers to the unfermented liquor run-off following extracting the grist during mashing.

In another aspect the invention relates to a method of preparing a fermented beverage such as beer comprising mixing any of the Bsp Man4 polypeptides of the invention with malt or adjunct.

Examples of beers comprise: full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, but also alternative cereal and malt beverages such as fruit flavoured malt beverages, e.g. citrus flavoured, such as lemon-, orange-, lime-, or berry-flavoured malt beverages, liquor flavoured malt beverages, e.g., vodka-, rum-, or tequila-flavoured malt liquor, or coffee flavoured malt beverages, such as caffeine-flavoured malt liquor, and the like.

One aspect of the invention relates to the use of any of the Bsp Man4 polypeptides according to the invention in the production of a fermented beverage, such as a beer.

Another aspect concerns a method of providing a fermented beverage comprising the step of contacting a mash and/or a wort with any of the Bsp Man4 polypeptides of the current invention.

A further aspect relates to a method of providing a fermented beverage comprising the steps of: (a) preparing a mash, (b) filtering the mash to obtain a wort, and (c) fermenting the wort to obtain a fermented beverage, such as a beer, wherein any of the Bsp Man4 polypeptides is added to: (i) the mash of step (a) and/or (ii) the wort of step (b) and/or (iii) the wort of step (c).

According to yet another aspect, a fermented beverage, such as a beer, is produced or provided by a method comprising the step(s) of (1) contacting a mash and/or a wort with any of the Bsp Man4 polypeptides of the current invention; and/or (2) (a) preparing a mash, (b) filtering the mash to obtain a wort, and (c) fermenting the wort to obtain a fermented beverage, such as a beer, wherein any of the Bsp Man4 polypeptides is added to: (i) the mash of step (a) and/or (ii) the wort of step (b) and/or (iii) the wort of step (c).

Particular embodiments pertains to any of the above use, method or fermented beverage, wherein said fermented beverage is a beer, such as full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, but also alternative cereal and malt beverages such as fruit flavoured malt beverages, e.g., citrus flavoured, such as lemon-, orange-, lime-, or berry-flavoured malt beverages, liquor flavoured malt beverages, e.g., vodka-, rum-, or tequila-flavoured malt liquor, or coffee flavoured malt beverages, such as caffeine-flavoured malt liquor, and the like.

### VIII. Bsp Man4 Polypeptides for Treating Coffee Extracts

The Bsp Man4 polypeptides described herein may also be used for hydrolyzing galactomannans present in liquid coffee extracts. In certain preferred embodiments, the Bsp Man4 polypeptides are used to inhibit gel formation during freeze drying of liquid coffee extracts. The decreased viscosity of the extract reduces the energy consumption during drying. In certain other preferred embodiments, the Bsp Man4 polypeptides are applied in an immobilized form in order to reduce enzyme consumption and avoid contamination of the coffee extract This use is further disclosed in EP 676 145.

In general terms the coffee extract is incubated in the presence of an isolated Bsp Man4 polypeptide or fragment or variant thereof under conditions suitable for hydrolyzing galactomannans present in liquid coffee extract.

### VIIIc Bsp Man4 Polypeptides for Use in Bakery Food Products

In another aspect the invention relates to a method of preparing baked products comprising addition of any of the Bsp Man4 polypeptides of the invention to dough, followed by baking the dough. Examples of baked products are well known to those skilled in the art and include breads, rolls, puff pastries, sweet fermented doughs, buns, cakes, crackers, cookies, biscuits, waffles, wafers, tortillas, breakfast cereals, extruded products, and the like.

Any of the Bsp Man4 polypeptides of the invention may be added to dough as part of a bread improver composition. Bread improvers are compositions containing a variety of ingredients, which improve dough properties and the quality of bakery products, e.g. bread and cakes. Bread improvers are often added in industrial bakery processes because of their beneficial effects e.g. the dough stability and the bread texture and volume. Bread improvers usually contain fats and oils as well as additives like emulsifiers, enzymes, antioxidants, oxidants, stabilizers and reducing agents. In addition to any of the Bsp Man4 polypeptides of the present invention, other enzymes which may also be present in the bread improver or which may be otherwise used in conjunction with any of the Bsp Man4 polypeptides of the present invention include amylases, hemicellulases, amylolytic complexes, lipases, proteases, xylanases, pectinases, pullulanases, non starch polysaccharide degrading enzymes and redox enzymes like glucose oxidase, lipoxygenase or ascorbic acid oxidase.

In a preferred bakery aspect of the current invention, any of the Bsp Man4 polypeptides of the invention may be added to dough as part of a bread improver composition which also comprises a glucomannan and/or galactomannan source such as konjac gum, guar gum, locust bean gum (Ceratonia siliqua), copra meal, ivory nut mannan (Phyteleohas macrocarpa), seaweed mannan extract, coconut meal, and the cell wall of brewers yeast (may be dried, or used in the form of brewers yeast extract). Other acceptable mannan derivatives for use in the current invention include unbranched β-1,4-linked mannan homopolymer and manno-oligosaccharides (mannobiose, mannotriose, mannotetraose and mannopentoase). The combination of any of the Bsp Man4 polypeptides of the invention with a glucomannan and/or galactomannan and/or galatoglucomannan further improves the dough tolerance, dough flexibility and dough stickiness, improves the bread crumb structure and retards staling of the bread, and the mannanase hydrolysates act as soluble prebiotics by promoting the growth of lactic acid bacteria commonly associated with good health when found at favourable population densities in the colon.

A further aspect of the invention relates to the use of any of the Bsp Man4 polypeptides of the invention in dough to improve dough tolerance, flexibility and stickiness. Preferably the dough to which any of the Bsp Man4 polypeptides of the invention may be added is not a pure white flour dough, but comprises bran or oat, rice, millet, maize, or legume flour in addition to or instead of pure wheat flour.

A yet further aspect of the invention relates to the use of any of the Bsp Man4 polypeptides of the invention in dough to improve the crumb structure and retard staling in the final baked product, such as bread.

### VIIIc Bsp Man4 Polypeptides for Use in Dairy Food Products

In one aspect of the current invention, any of the Bsp Man4 polypeptides of the invention may be added to milk or any other dairy product to which has also been added a glucomannan and/or galactomannan. Typical glucomannan and/or galactomannan sources are listed above in the bakery aspects, and include guar or konjac gum. The combination of any of the Bsp Man4 polypeptides of the invention with a glucomannan and/or galactomannan releases mannanase hydrolysates (mannooligosaccharides) which act as soluble prebiotics by promoting the selective growth and proliferation of probiotic bacteria (especially Bifidobacteria and Lactobacillus lactic acid bacteria) commonly associated with good health when found at favourable population densities in the large intestine or colon.

In another aspect the invention relates to a method of preparing milk or dairy products comprising addition of any of the Bsp Man4 polypeptides of the invention and addition of any glucomannan or galactomannan or galactoglucomannan.

In another aspect of the invention any of the Bsp Man4 polypeptides of the invention are used in combination with any glucomannan or galactomannan prior to or following addition to a dairy based foodstuff to produce a dairy based foodstuff comprising prebiotic mannan hydrolysates. In a further aspect of the invention the thus produced mannooligosacharide-containing dairy product is capable of increasing the population of beneficial human intestinal microflora, and in a yet further aspect of the current invention the dairy based foodstuff may comprise any of the Bsp Man4 polypeptides of the current invention together with any source of glucomannan and/or galactomannan and/or galactoglucomannan, and a dose sufficient for inoculation of at least one strain of bacteria (such as Bifidobacteria or Lactobacillus) known to be of benefit in the human large intestine. Preferably said dairy-based foodstuff is a yoghurt or milk drink.

### IX. Bsp Man4 Polypeptides for Paper Pulp Bleaching

The Bsp Man4 polypeptides described herein find further use in the enzyme aided bleaching of paper pulps such as chemical pulps, semi-chemical pulps, kraft pulps, mechanical pulps or pulps prepared by the sulfite method. In general terms, paper pulps are incubated with an isolated Bsp Man4 polypeptide or fragment or variant thereof under conditions suitable for bleaching the paper pulp.

In some embodiments, the pulps are chlorine free pulps bleached with oxygen, ozone, peroxide or peroxyacids. In some embodiments, the Bsp Man4 polypeptides are used in enzyme aided bleaching of pulps produced by modified or continuous pulping methods that exhibit low lignin contents. In some other embodiments, the Bsp Man4 polypeptides are applied alone or preferably in combination with xylanase and/or endoglucanase and/or alpha-galactosidase and/or cellobiohydrolase enzymes.

### X. Bsp Man4 Polypeptides for Degrading Thickeners

Galactomannans such as guar gum and locust bean gum are widely used as thickening agents e.g., in food and print paste for textile printing such as prints on T-shirts. Thus the Bsp Man4 polypeptides described herein also find use in reducing the thickness or viscosity of mannan-containing substrates. In certain embodiments, the Bsp Man4 polypeptides described herein are used for reducing the viscosity of residual food in processing equipment and thereby facilitate cleaning after processing. In certain other embodiments, the disclosed Bsp Man4 polypeptides are used for reducing viscosity of print paste, thereby facilitating wash out of surplus print paste after textile printings. In general terms, a mannan-containing substrate is incubated with an isolated Bsp Man4 polypeptide or fragment or variant thereof under conditions suitable for reducing the viscosity of the mannan-containing substrate.

Other aspects and embodiments of the present compositions and methods will be apparent from the foregoing description and following examples.

### EXAMPLES

The following examples are provided to demonstrate and illustrate certain preferred embodiments and aspects of the present disclosure and should not be construed as limiting.

In the experimental disclosure which follows, the following abbreviations apply: M (molar); mM (millimolar); μM (micromolar); nM (nanomolar); mol (moles); mmol (millimoles); μmol (micromoles); nmol (nanomoles); g and gm (grams); mg (milligrams); μg (micrograms); pg (picograms); L (liters); ml and mL (milliliters); μl and μL (microliters); cm (centimeters); mm (millimeters); μm (micrometers); nm (nanometers); U (units); MW (molecular weight); sec (seconds); min(s) (minute/minutes); h(s) and hr(s) (hour/hours); ° C. (degrees Centigrade); QS (quantity sufficient); ND (not done); rpm (revolutions per minute); H₂O (water); dH₂O (deionized water); HCl (hydrochloric acid); aa (amino acid); by (base pair); kb (kilobase pair); kD (kilodaltons); MgCl₂ (magnesium chloride); NaCl (sodium chloride); Ca (calcium); Mg (magnesium); HEPES (4-(2-hydroxyethyl)-1-piperazineethanesulfonic acid); CHES (N-cyclohexyl-2-aminoethanesulfonic acid); w/v (weight to volume); v/v (volume to volume); g (gravity); OD (optical density); ppm (parts per million); m-(meta-); o-(ortho-); p-(para-); PAHBAH (p-hydroxybenzoic acid hydrazide); Bsp Man4 (Bacillus sp. mannanase4); SRI (stain removal index); and % SR (percentage stain removal).

### Example 1

### Cloning of the Bacillus sp. SWT81 Glycosyl Hydrolase Bsp Man4

Bacillus sp. SWT81 was selected as a potential source for various glycosyl hydrolases and other enzymes, useful for industrial applications. Genomic DNA for sequencing was obtained by first growing Bacillus sp. SWT81 on GAM agar plates (Jones et al., IJSEM, 55:1711-1714, 2005) at 37° C. for 24 h. Cell material was scraped from the plates and used to prepare genomic DNA using the ZF Fungal/Bacterial DNA miniprep kit from Zymo (Cat No. D6005). The genomic DNA was used for genome sequencing and to amplify the Bsp Man4 gene for expression cloning. The entire genome of the Bacillus sp. SWT81 strain was sequenced using Illumina® sequencing by synthesis (SBS) technology (www.baseclear.com/sequencing/illumina-sequencing/). Genome sequencing and assembly of the sequence data was performed by BaseClear (Leiden, The Netherlands). Contigs were annotated by BioXpr (Namur, Belgium). One of the genes identified this way in Bacillus sp. SWT81 encodes a glycosyl hydrolase with homology to mannanases of various other bacteria as determined from a BLAST search (Altschul et al., J Mol Biol, 215: 403-410, 1990). The sequence of this gene, called the Bsp Man4 gene, is depicted as SEQ ID NO.1. The protein encoded by the Bsp Man4 gene is depicted as SEQ ID NO. 2. At the N-terminus, Bsp Man4 has a 29 amino acid signal peptide as predicted by SignalP-3.0 program (www.cbs.dtu/services/SignalP) set to SignalP-NN system (Emanuelsson et al., Nature Protocols, 2:953-971, 2007). This indicates that Bsp Man4 is a secreted glycosyl hydrolase.

The nucleotide sequence of the Bsp Man4 coding region is set forth as SEQ ID NO:1. The coding region of the predicted signal peptide sequence is shown in italics.

atgggaacatggaaaaaggggtttgtgttatttattgtcctaatgttagtttttgatgtatcgatgttgggtgtaaatgtaagcgcttcac
aagaagggcgtcaacttaacatggcagatgaggatgcttcaaagtatacgaaggagttatttgcttttcttcaagatgtaagtggttcaca
agtgttatttggacaacagcatgcaacagatgaaggattaactttaacaaatccagctccaagaacaggttccactcaatctgaagttttc
aatgcagttggggattatccagctgtgtttggatgggacacgaatagcctagatggtcgtgaaaagcctggcattgcaggtaatgtagaac
aaagtataaaaaatacggctcagtccatgaaagtggctcatgatttaggagggattattacactaagcatgcacccagataattttgtaac
agggggtccttatggtgatacaacagggaatgttgtaaaagaaattcttccaggtggatcaaaacatgcagagtttaacgcgtggttggac
aatattgctgcgcttgctcacgagctgaaagatgagaatggtgaacctattccgatgatttttcggccattccatgaacaaacaggatctt
ggttttggtggggagcaagcacaacttcacccgaacaatataaagcgatttttcgttatacagtagaatatttgcgagatgttaaaggcgt
aaataatattttatatggcttttcacctggggcgggacctgctggagatgtaaatcgctatttagaaacatatccaggggatgattacgtt
gatattttcggtattgacaattatgacaataaagacaatgcagggtcagaagcttggttaagtggtatggtcaaagacttggcgatgatta
gccgattagctgaacaaaaagaaaaagtagcggcttttactgagtatgggtacagtgcaaccggaattaatcgtcaagggaatacattaga
ctggtacacacgtgtattagatgcgattgctgctgatgaagacgcacgtaaaatatcatacatgttgacatgggcgaactttggttggccg
aataatatgtatgttccttatcgtgatatccacaatgaattaggtggagaccatgagttattaccggactttgaagctttccatgcggatg
actacacagcatttcgagatgagataaaaggaaagatatataatactggaaaggaatataccgtttctcctcatgagccgtttatgtatgt
tatatctccgattacaggttctacagtgacaagcgaaacggtaacaatccaagcaaaagtagcgaatgacgaacacgcaagagtcactttc
agggtcgatggttctagtttggaagaagaaatggttttcaatgatgacactttatattatacaggttcttttacaccagatgcagcagtga
atggcggagctgttgatgtgattgtagcttattattctagtggagaaaaagtccaagaagaaacaattcgtttatttgtaaaaattcctga
aatgtctttgttaacattaacgtttgatgatgatataaacggaatcaaaagcaatggaacatggcctgaagatggtgtaacatctgaaatt
gaccacgctattgtagatggagacggcaagttgatgttctctgttcaaggaatgtcacctactgaaacatggcaagagctcaagttagaat
taacagaactatcagatgtgaacattgatgcggttaagaaaatgaagtttgacgcgcttatcccagcaggtagtgaagaaggttcagtcca
aggaatcgtacaacttccaccggattgggagacgaaatatgggatgaatgaaacaacgaagtcaataaaagacttagagactgttactgtt
aatggaagcgattataaacggttggaagtgactgtttctatcgacaatcaaggaggagctacaggaatcgctttatcattagtaggatccc
aactcgatttgttagaacctgtctacatcgataatattgaacttctaaattcctttgaagcaccaccagcagattcttttcttgttgatga
ttttgaaggttattttggggatgacacgttgttacatcgcaattattctagcaatggagatccaattacactatcgttaacaagtgagttt
aaaaataatggagaatttggattgaagtatgattattcgattggctcgatgggttatgcagggaggcaaacatcactaggacctgtcgatt
ggagcggagctaatgcttttgaattttggatgaaacatggacaacttgaagggaatcatttaactgtacaaattcgaataggtgatgttag
ctttgaaaaaaatcttgaattaatggatgctcatgaaggtgtagtgacaatcccgttttctgaatttgctccagctgcttgggaaaataag
cctggcgttatcattgacgaacaaaaattgaaaagagtgagtcaatttgctctttacacaggcggggctagacaatctggaacaatctact
ttgatgatttacgagcggtatatgatgaaagtttaccatcagttccagttccgaaagaggaggaagaggaaaaagaggtcgctcctattat
ttatcattttgaatctggaattgataattgggaagggggacaagcaacacatagcaatgggcacctcaaagtaacggttcgtttaggtgaa
ggtcagcaaaccgaagtgaagaaaacatcaaattataatttaacagggtataattatatagtagctaatataaaacatgacgatacaggaa
tgtttggtagtgacccgcttcaagtgaaaatctttacgaaagcaggaggttgggtatgggctgattcaggaaatcaaccgatttactccga
cgattatactcaagttgtgtatgatattactactttagctaacaaaaatgcagtccaagaaatcgggtttgaatttttggctccttcaggt
tcttcagggacgacgaatcctttcatagattcagtagcgattgttacgagtctcgatcaattgtctgagcagccagagcagccagaacaac
caggaacaccagatactgatgataataaagaggataaagatagaagaaatgtagaagtgaacgaggaaggacaaaaactacccaaaacagc
aacgtcaatatttaattatttgctaattggttttgtttttgtagggattggatttagtctatttatttataaaagaagaaaaacagtg.

The amino acid sequence of the protein encoded by the Bsp Man 4 gene is set forth as SEQ ID NO:2. The predicted signal peptide is shown in italics.

MGTWKKGFVLFIVLMLVFDVSMLGVNVSASQEGRQLNMADEDASKYTKEL
FAFLQDVSGSQVLFGQQHATDEGLTLTNPAPRTGSTQSEVFNAVGDYPAV
FGWDTNSLDGREKPGIAGNVEQSIKNTAQSMKVAHDLGGIITLSMHPDNF
VTGGPYGDTTGNVVKEILPGGSKHAEFNAWLDNIAALAHELKDENGEPIP
MIFRPFHEQTGSWFWWGASTTSPEQYKAIFRYTVEYLRDVKGVNNILYGF
SPGAGPAGDVNRYLETYPGDDYVDIFGIDNYDNKDNAGSEAWLSGMVKDL
AMISRLAEQKEKVAAFTEYGYSATGINRQGNTLDWYTRVLDAIAADEDAR
KISYMLTWANFGWPNNMYVPYRDIHNELGGDHELLPDFEAFHADDYTAFR
DEIKGKIYNTGKEYTVSPHEPFMYVISPITGSTVTSETVTIQAKVANDEH
ARVTFRVDGSSLEEEMVFNDDTLYYTGSFTPDAAVNGGAVDVIVAYYSSG
EKVQEETIRLFVKIPEMSLLTLTFDDDINGIKSNGTWPEDGVTSEIDHAI
VDGDGKLMFSVQGMSPTETWQELKLELTELSDVNIDAVKKMKFDALIPAG
SEEGSVQGIVQLPPDWETKYGMNETTKSIKDLETVTVNGSDYKRLEVTVS
IDNQGGATGIALSLVGSQLDLLEPVYIDNIELLNSFEAPPADSFLVDDFE
GYFGDDTLLHRNYSSNGDPITLSLTSEFKNNGEFGLKYDYSIGSMGYAGR
QTSLGPVDWSGANAFEFWMKHGQLEGNHLTVQIRIGDVSFEKNLELMDAH
EGVVTIPFSEFAPAAWENKPGVIIDEQKLKRVSQFALYTGGARQSGTIYF
DDLRAVYDESLPSVPVPKEEEEEKEVAPIIYHFESGIDNWEGGQATHSNG
HLKVTVRLGEGQQTEVKKTSNYNLTGYNYIVANIKHDDTGMFGSDPLQVK
IFTKAGGWVWADSGNQPIYSDDYTQVVYDITTLANKNAVQEIGFEFLAPS
GSSGTTNPFIDSVAIVTSLDQLSEQPEQPEQPGTPDTDDNKEDKDRRNVE
VNEEGQKLPKTATSIFNYLLIGFVFVGIGFSLFIYKRRKTV.

### Example 2

### Expression of Bacillus sp. Glycosyl Hydrolase (Bsp Man4)

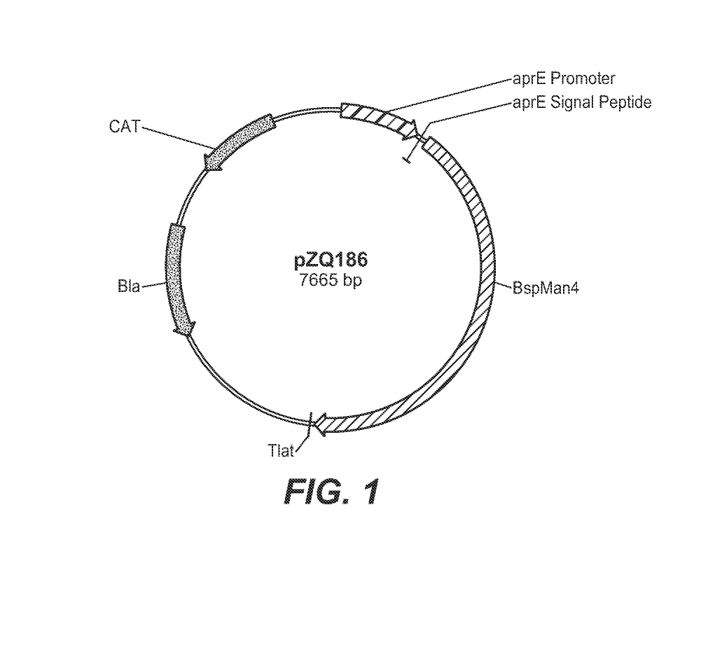

The Bsp Man4 gene was amplified from genomic DNA of Bacillus sp. using the following primers: Primer 1(BssHII) 5′-TGAGCGCGCA GGCAGCTGGT AAATCACAAG AAGGGCGTCA ACT-3′ (SEQ ID NO:3), and Primer 2 (XhoI) 5′-CGCCTCGAGT TACACTGTTT TTCTTCTTTT AT-3′ (SEQ ID NO:4). After digestion with BssHII/XhoI, the PCR product was cloned into the p2JM103BBI expression vector (Vogtentanz, Protein Expr Purif, 55:40-52, 2007) digested with the same restriction enzymes. Ligation of this DNA fragment to the PCR amplified gene encoding the Bsp Man4 mature protein resulted in the addition of three codons from the 3′ end of the nucleic acid encoding the Bacillus subtilis AprE pro-peptide to the 5′ end of the coding region of the mature form of Bsp Man4. The resulting plasmid was labeled pZQ186 (aprE-Bsp Man4). A plasmid map of pZQ186 is provided as FIG. 1. Following the natural signal peptidase cleavage in the host, the recombinant Bsp Man4 protein produced in this manner has three additional amino acids (Ala-Gly-Lys) at its amino-terminus. The sequence of Bsp Man4 gene was confirmed by DNA sequencing (SEQ ID NO:5).

The Bsp Man4 protein was produced in Bacillus subtilis cells using previously described methods (Vogtentanz, Protein Expr Purif, 55:40-52, 2007). The protein was secreted into the extracellular medium and filtered culture medium was used to perform the cleaning assay and the pH and temperature profile experiments. The dosing was based on total protein determined by a Bradford type assay using the Biorad protein assay (500-0006EDU) and corrected for purity as determined by SDS-PAGE using a Criterion stain free system from Bio-Rad).

Analysis of the culture supernatant on an SDS-PAGE gel revealed three separate and distinct protein bands falling within the expected molecular weight range of Bsp Man4. The concentrated culture supernatant of Bacillus cells expressing Bsp Man4 was used for purification using three chromatography columns. Concentrated culture supernatant buffered in 20 mM Tris pH 7.5 was loaded on an anion exchange Sepharose column (Sepharose-Q FF, XK 16/10) equilibrated with 20 mM Tris pH 7.5. The protein was eluted from the column using a linear gradient of equilibration/wash buffer to 20 mM Tris, pH 7.5 buffer containing 0.5 M NaCl. The pooled sample was adjusted to final concentration of 1M (NH₄)₂SO₄ and loaded onto a hydrophobic interaction chromatography column (HiLoad Phenyl HP, 16/10) equilibrated with 20 mM sodium phosphate pH 6.0, 1M (NH₄)₂SO₄ buffer. The protein was eluted from the column using a linear gradient of equilibration/wash buffer to 20 mM sodium phosphate pH 6.0. Three fractions, approximately 100 kD (alpha), 70 kD (beta), and 50 kD (gamma), all with mannanase activity, were collected separately. Each fraction was loaded onto the gel filtration HiLoad Superdex 75 pg 26/60 column (for 50 kD and 70 kD fractions) or HiLoad Superdex 200 pg 26/60 column (for 100 kD fraction), and the mobile phase used was 20 mM sodium phosphate, pH 7.0, containing 0.15 M NaCl. The pooled samples from gel filtration columns were concentrated to obtain purified protein samples.

Nucleotide sequence of Bsp Man4 gene from expression plasmid pZQ186 is set forth as SEQ ID NO:5. The aprE signal sequence is shown in italics.

gtgagaagcaaaaaattgtggatcagcttgttgtttgcgttaacgttaatctttacgatggcgttcagcaacatgagcgcgcaggcagct
ggtaaatcacaagaagggcgtcaacttaacatggcagatgaggatgcttcaaagtatacgaaggagttatttgcttttcttcaagatgta
agtggttcacaagtgttatttggacaacagcatgcaacagatgaaggattaactttaacaaatccagctccaagaacaggttccactcaa
tctgaagttttcaatgcagttggggattatccagctgtgtttggatgggacacgaatagcctagatggtcgtgaaaagcctggcattgca
ggtaatgtagaacaaagtataaaaaatacggctcagtccatgaaagtggctcatgatttaggagggattattacactaagcatgcaccca
gataattttgtaacagggggtccttatggtgatacaacagggaatgttgtaaaagaaattcttccaggtggatcaaaacatgcagagttt
aacgcgtggttggacaatattgctgcgcttgctcacgagctgaaagatgagaatggtgaacctattccgatgatttttcggccattccat
gaacaaacaggatcttggttttggtggggagcaagcacaacttcacccgaacaatataaagcgatttttcgttatacagtagaatatttg
cgagatgttaaaggcgtaaataatattttatatggcttttcacctggggcgggacctgctggagatgtaaatcgctatttagaaacatat
ccaggggatgattacgttgatattttcggtattgacaattatgacaataaagacaatgcagggtcagaagcttggttaagtggtatggtc
aaagacttggcgatgattagccgattagctgaacaaaaagaaaaagtagcggcttttactgagtatgggtacagtgcaaccggaattaat
cgtcaagggaatacattagactggtacacacgtgtattagatgcgattgctgctgatgaagacgcacgtaaaatatcatacatgttgaca
tgggcgaactttggttggccgaataatatgtatgttccttatcgtgatatccacaatgaattaggtggagaccatgagttattaccggac
tttgaagctttccatgcggatgactacacagcatttcgagatgagataaaaggaaagatatataatactggaaaggaatataccgtttct
cctcatgagccgtttatgtatgttatatctccgattacaggttctacagtgacaagcgaaacggtaacaatccaagcaaaagtagcgaat
gacgaacacgcaagagtcactttcagggtcgatggttctagtttggaagaagaaatggttttcaatgatgacactttatattatacaggt
tcttttacaccagatgcagcagtgaatggcggagctgttgatgtgattgtagcttattattctagtggagaaaaagtccaagaagaaaca
attcgtttatttgtaaaaattcctgaaatgtctttgttaacattaacgtttgatgatgatataaacggaatcaaaagcaatggaacatgg
cctgaagatggtgtaacatctgaaattgaccacgctattgtagatggagacggcaagttgatgttctctgttcaaggaatgtcacctact
gaaacatggcaagagctcaagttagaattaacagaactatcagatgtgaacattgatgcggttaagaaaatgaagtttgacgcgcttatc
ccagcaggtagtgaagaaggttcagtccaaggaatcgtacaacttccaccggattgggagacgaaatatgggatgaatgaaacaacgaag
tcaataaaagacttagagactgttactgttaatggaagcgattataaacggttggaagtgactgtttctatcgacaatcaaggaggagct
acaggaatcgctttatcattagtaggatcccaactcgatttgttagaacctgtctacatcgataatattgaacttctaaattcctttgaa
gcaccaccagcagattcttttcttgttgatgattttgaaggttattttggggatgacacgttgttacatcgcaattattctagcaatgga
gatccaattacactatcgttaacaagtgagtttaaaaataatggagaatttggattgaagtatgattattcgattggctcgatgggttat
gcagggaggcaaacatcactaggacctgtcgattggagcggagctaatgcttttgaattttggatgaaacatggacaacttgaagggaat
catttaactgtacaaattcgaataggtgatgttagctttgaaaaaaatcttgaattaatggatgctcatgaaggtgtagtgacaatcccg
ttttctgaatttgctccagctgcttgggaaaataagcctggcgttatcattgacgaacaaaaattgaaaagagtgagtcaatttgctctt
tacacaggcggggctagacaatctggaacaatctactttgatgatttacgagcggtatatgatgaaagtttaccatcagttccagttccg
aaagaggaggaagaggaaaaagaggtcgctcctattatttatcattttgaatctggaattgataattgggaagggggacaagcaacacat
agcaatgggcacctcaaagtaacggttcgtttaggtgaaggtcagcaaaccgaagtgaagaaaacatcaaattataatttaacagggtat
aattatatagtagctaatataaaacatgacgatacaggaatgtttggtagtgacccgcttcaagtgaaaatctttacgaaagcaggaggt
tgggtatgggctgattcaggaaatcaaccgatttactccgacgattatactcaagttgtgtatgatattactactttagctaacaaaaat
gcagtccaagaaatcgggtttgaatttttggctccttcaggttcttcagggacgacgaatcctttcatagattcagtagcgattgttacg
agtctcgatcaattgtctgagcagccagagcagccagaacaaccaggaacaccagatactgatgataataaagaggataaagatagaaga
aatgtagaagtgaacgaggaaggacaaaaactacccaaaacagcaacgtcaatatttaattatttgctaattggttttgtttttgtaggg
attggatttagtctatttatttataaaagaagaaaaacagtg.

The amino acid sequence of Bsp Man4 expressed from plasmid pZQ186 is set forth as SEQ ID NO:6. The signal sequence is shown in italics, while the three amino acid amino-terminal extension is shown in bold.

MRSKKLWISLLFALTLIFTMAFSNMSAQAAGKSQEGRQLNMADEDASKYT
KELFAFLQDVSGSQVLFGQQHATDEGLTLTNPAPRTGSTQSEVFNAVGDY
PAVFGWDTNSLDGREKPGIAGNVEQSIKNTAQSMKVAHDLGGIITLSMHP
DNFVTGGPYGDTTGNVVKEILPGGSKHAEFNAWLDNIAALAHELKDENGE
PIPMIFRPFHEQTGSWFWWGASTTSPEQYKAIFRYTVEYLRDVKGVNNIL
YGFSPGAGPAGDVNRYLETYPGDDYVDIFGIDNYDNKDNAGSEAWLSGMV
KDLAMISRLAEQKEKVAAFTEYGYSATGINRQGNTLDWYTRVLDAIAADE
DARKISYMLTWANFGWPNNMYVPYRDIHNELGGDHELLPDFEAFHADDYT
AFRDEIKGKIYNTGKEYTVSPHEPFMYVISPITGSTVTSETVTIQAKVAN
DEHARVTFRVDGSSLEEEMVFNDDTLYYTGSFTPDAAVNGGAVDVIVAYY
SSGEKVQEETIRLFVKIPEMSLLTLTFDDDINGIKSNGTWPEDGVTSEID
HAIVDGDGKLMFSVQGMSPTETWQELKLELTELSDVNIDAVKKMKFDALI
PAGSEEGSVQGIVQLPPDWETKYGMNETTKSIKDLETVTVNGSDYKRLEV
TVSIDNQGGATGIALSLVGSQLDLLEPVYIDNIELLNSFEAPPADSFLVD
DFEGYFGDDTLLHRNYSSNGDPITLSLTSEFKNNGEFGLKYDYSIGSMGY
AGRQTSLGPVDWSGANAFEFWMKHGQLEGNHLTVQIRIGDVSFEKNLELM
DAHEGVVTIPFSEFAPAAWENKPGVIIDEQKLKRVSQFALYTGGARQSGT
IYFDDLRAVYDESLPSVPVPKEEEEEKEVAPIIYHFESGIDNWEGGQATH
SNGHLKVTVRLGEGQQTEVKKTSNYNLTGYNYIVANIKHDDTGMFGSDPL
QVKIFTKAGGWVWADSGNQPIYSDDYTQVVYDITTLANKNAVQEIGFEFL
APSGSSGTTNPFIDSVAIVTSLDQLSEQPEQPEQPGTPDTDDNKEDKDRR
NVEVNEEGQKLPKTATSIFNYLLIGFVFVGIGFSLFIYKRRKTV.

The amino acid sequence of the mature form of Bsp Man4 is set forth as SEQ ID NO:7. The three amino acid N-terminal extension is shown in bold.

AGKSQEGRQLNMADEDASKYTKELFAFLQDVSGSQVLFGQQHATDEGLTL
TNPAPRTGSTQSEVFNAVGDYPAVFGWDTNSLDGREKPGIAGNVEQSIKN
TAQSMKVAHDLGGIITLSMHPDNFVTGGPYGDTTGNVVKEILPGGSKHAE
FNAWLDNIAALAHELKDENGEPIPMIFRPFHEQTGSWFWWGASTTSPEQY
KAIFRYTVEYLRDVKGVNNILYGFSPGAGPAGDVNRYLETYPGDDYVDIF
GIDNYDNKDNAGSEAWLSGMVKDLAMISRLAEQKEKVAAFTEYGYSATGI
NRQGNTLDWYTRVLDAIAADEDARKISYMLTWANFGWPNNMYVPYRDIHN
ELGGDHELLPDFEAFHADDYTAFRDEIKGKIYNTGKEYTVSPHEPFMYVI
SPITGSTVTSETVTIQAKVANDEHARVTFRVDGSSLEEEMVFNDDTLYYT
GSFTPDAAVNGGAVDVIVAYYSSGEKVQEETIRLFVKIPEMSLLTLTFDD
DINGIKSNGTWPEDGVTSEIDHAIVDGDGKLMFSVQGMSPTETWQELKLE
LTELSDVNIDAVKKMKFDALIPAGSEEGSVQGIVQLPPDWETKYGMNETT
KSIKDLETVTVNGSDYKRLEVTVSIDNQGGATGIALSLVGSQLDLLEPVY
IDNIELLNSFEAPPADSFLVDDFEGYFGDDTLLHRNYSSNGDPITLSLTS
EFKNNGEFGLKYDYSIGSMGYAGRQTSLGPVDWSGANAFEFWMKHGQLEG
NHLTVQIRIGDVSFEKNLELMDAHEGVVTIPFSEFAPAAWENKPGVIIDE
QKLKRVSQFALYTGGARQSGTIYFDDLRAVYDESLPSVPVPKEEEEEKEV
APIIYHFESGIDNWEGGQATHSNGHLKVTVRLGEGQQTEVKKTSNYNLTG
YNYIVANIKHDDTGMFGSDPLQVKIFTKAGGWVWADSGNQPIYSDDYTQV
VYDITTLANKNAVQEIGFEFLAPSGSSGTTNPFIDSVAIVTSLDQLSEQP
EQPEQPGTPDTDDNKEDKDRRNVEVNEEGQKLPKTATSIFNYLLIGFVFV
GIGFSLFIYKRRKTV.

The amino acid sequence of the mature form of Bsp Man4 based on the naturally occurring gene sequence is set forth as SEQ ID NO:8.

SQEGRQLNMADEDASKYTKELFAFLQDVSGSQVLFGQQHATDEGLTLTNP
APRTGSTQSEVFNAVGDYPAVFGWDTNSLDGREKPGIAGNVEQSIKNTAQ
SMKVAHDLGGIITLSMHPDNFVTGGPYGDTTGNVVKEILPGGSKHAEFNA
WLDNIAALAHELKDENGEPIPMIFRPFHEQTGSWFWWGASTTSPEQYKAI
FRYTVEYLRDVKGVNNILYGFSPGAGPAGDVNRYLETYPGDDYVDIFGID
NYDNKDNAGSEAWLSGMVKDLAMISRLAEQKEKVAAFTEYGYSATGINRQ
GNTLDWYTRVLDAIAADEDARKISYMLTWANFGWPNNMYVPYRDIHNELG
GDHELLPDFEAFHADDYTAFRDEIKGKIYNTGKEYTVSPHEPFMYVISPI
TGSTVTSETVTIQAKVANDEHARVTFRVDGSSLEEEMVFNDDTLYYTGSF
TPDAAVNGGAVDVIVAYYSSGEKVQEETIRLFVKIPEMSLLTLTFDDDIN
GIKSNGTWPEDGVTSEIDHAIVDGDGKLMFSVQGMSPTETWQELKLELTE
LSDVNIDAVKKMKFDALIPAGSEEGSVQGIVQLPPDWETKYGMNETTKSI
KDLETVTVNGSDYKRLEVTVSIDNQGGATGIALSLVGSQLDLLEPVYIDN
IELLNSFEAPPADSFLVDDFEGYFGDDTLLHRNYSSNGDPITLSLTSEFK
NNGEFGLKYDYSIGSMGYAGRQTSLGPVDWSGANAFEFWMKHGQLEGNHL
TVQIRIGDVSFEKNLELMDAHEGVVTIPFSEFAPAAWENKPGVIIDEQKL
KRVSQFALYTGGARQSGTIYFDDLRAVYDESLPSVPVPKEEEEEKEVAPI
IYHFESGIDNWEGGQATHSNGHLKVTVRLGEGQQTEVKKTSNYNLTGYNY
IVANIKHDDTGMFGSDPLQVKIFTKAGGWVWADSGNQPIYSDDYTQVVYD
ITTLANKNAVQEIGFEFLAPSGSSGTTNPFIDSVAIVTSLDQLSEQPEQP
EQPGTPDTDDNKEDKDRRNVEVNEEGQKLPKTATSIFNYLLIGFVFVGIG
FSLFIYKRRKTV.

### Example 3

### pH Profile of Bsp Man4

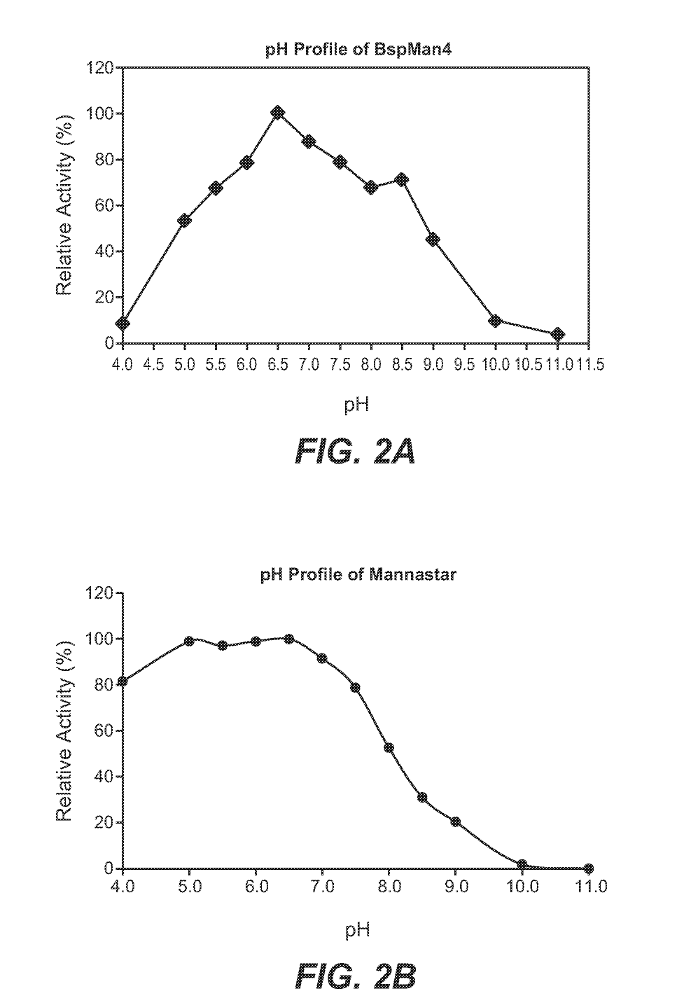

The pH profile of Bsp Man4 was determined using the beta-mannazyme tablet assay from Megazyme (Tmnz 1/02; Azurine-crosslinked carob galactomannan) with minor modifications to the suggested protocol. The assay was performed in 50 mm Acetate/Bis-Tris/HEPES/CHES buffer adjusted to pH values between 4 and 11. The enzyme solution was diluted into the assay buffer and 500 μl of the enzyme solution was equilibrated at 40° C. before adding one substrate tablet. After 10 minutes, the reaction was stopped by adding 10 ml 2% Tris pH 12. The tubes were left at room temperature for 5 minutes, stirred and the liquid filtered through a Whatman No.1 paper filter. Release of blue dye from the substrate was quantified by measuring the optical density at 590 nm. Enzyme activity at each pH was reported as relative activity where the activity at the pH optimum was set to 100%. The pH profile of Bsp Man4 is shown in FIG. 2A. Bsp Man 4 was found to have an optimum pH at about 6.5, and was found to retain greater than 70% of maximum activity between pH 6.0 and 8.5.

The pH profile of Mannastar™ was studied by assaying for mannanase activity at varying pH values ranging from 4-11 using the beta-mannazyme tablet assay (Megazyme, Ireland). The generation of water soluble dye fragments was monitored after 10 min at OD 590 nm at each pH value. A pH profile plot was made by setting the highest OD value for activity to 100 and determining the activity at the other pH values relative to the highest OD value. The pH profile of Mannastar™ is shown in FIG. 2B. Mannastar™ was found to retain greater than 70% of maximum activity between pH 4 and 7.5.

### Example 4

### Temperature Profile of Bsp Man4

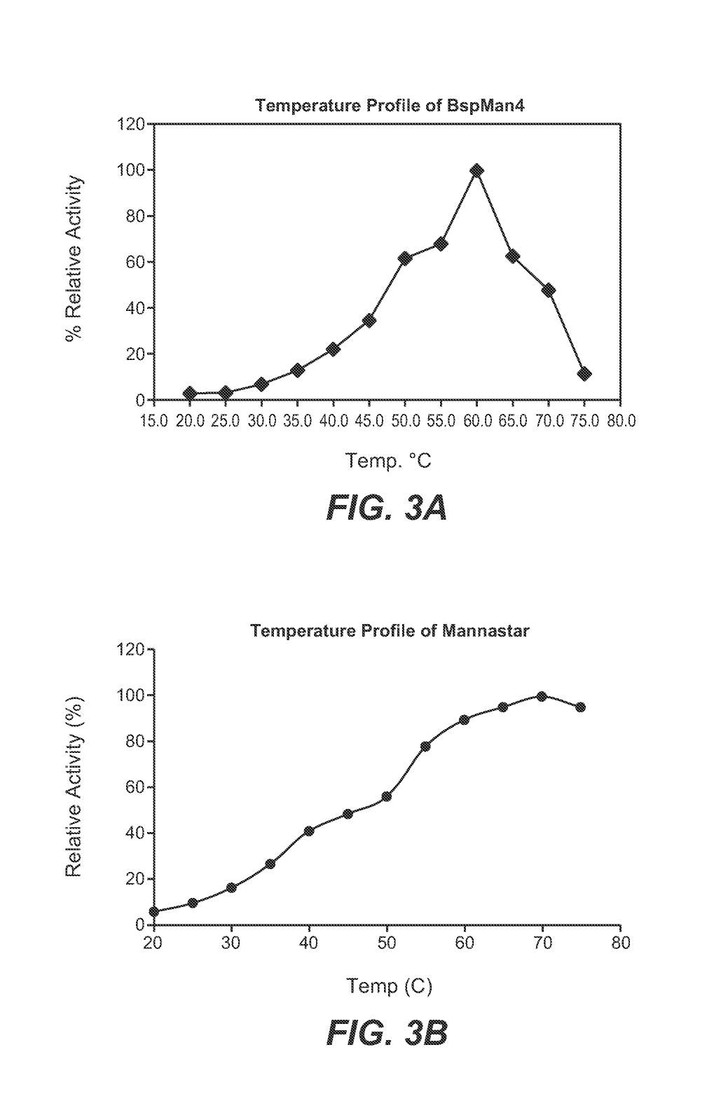

The temperature optimum of purified Bsp Man4 was determined by assaying for mannanase activity at temperatures varying between 20° C. and 75° C. for 10 minutes in 50 mM sodium citrate buffer at pH 6. The activity was reported as relative activity where the activity at the temperature optimum was set to 100%. The temperature profile of Bsp Man4 is shown in FIG. 3A. Bsp Man 4 was found to have an optimum temperature of about 60° C., and was found to retain greater than 70% of maximum activity between 55° C. and 65° C.

The temperature profile of Mannastar™ was studied by assaying for mannanase activity at varying temperatures ranging from 20° C. to 75° C. using the beta-mannazyme tablet assay (Megazyme, Ireland) in 50 mM sodium acetate buffer at pH 6. The generation of water soluble dye fragments was monitored after 10 min at OD 590 nm at each temperature. The temperature profile was made by setting the highest OD value for activity to 100% and determining the activity at the other temperatures relative to the maximum. The temperature profile of Mannastar™ is shown in FIG. 3B. Mannastar™ was found to retain greater than 70% maximum activity 55° C. and 75° C.

### Example 5

### Mannanase Activity of Bsp Man4

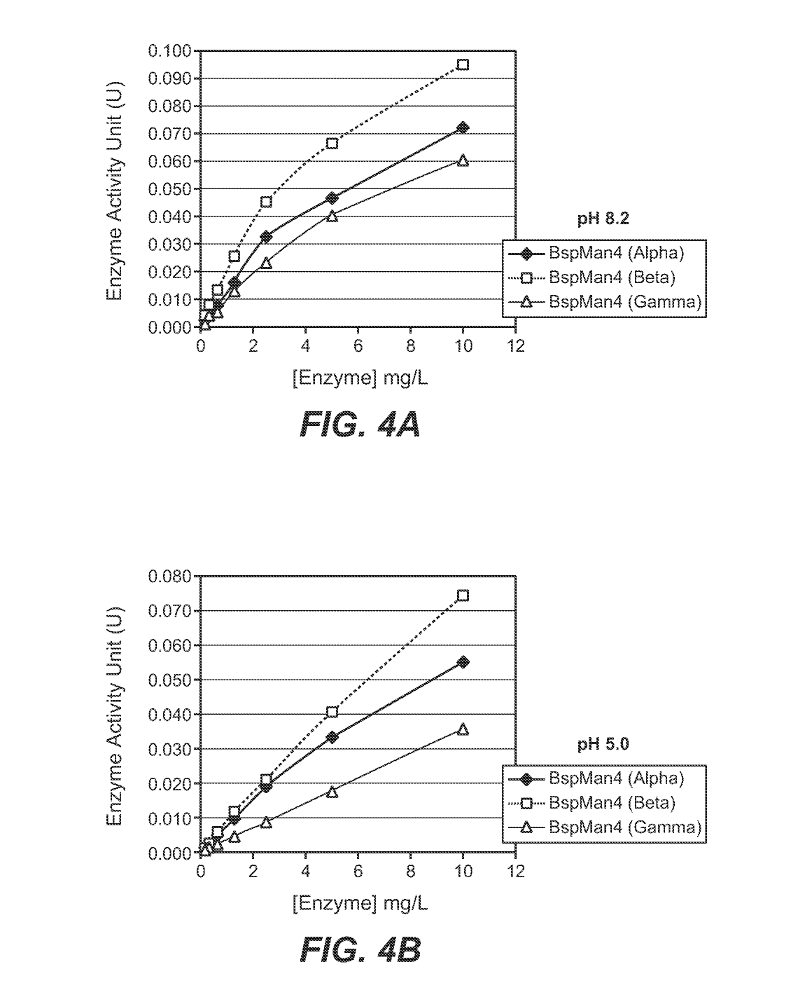

Bsp Man4 (EC number 3.2.1.78) belongs to the CAZy number GH26 glycosyl hydrolase family. Three forms of Bsp Man4 were identified by SDS-PAGE: alpha (MW˜100 kDa); beta (MW˜70 kDa); and gamma (MW˜50 kDa). The sample containing the gamma form of Bsp Man4 was a mixture including unrelated proteins, with Bsp Man4 present at 30% w/w. The beta 1-4 mannanase activity of the three forms of Bsp Man4 was measured using 1% Megazyme Low Viscosity Carob Galactomannan (Megazyme International, Ireland) as a substrate in a PAHBAH assay (Lever, Anal Biochem, 47:248, 1972). The assay was performed either in 50 mM sodium acetate pH 5, 0.005% Tween-80 buffer at 50° C. for 10 minutes or 50 mM HEPES pH 8.2, 0.005% Tween-80 buffer at 30° C. for 30 minutes. A standard curve using mannose was created for each buffer and used to calculate enzyme activity units. Enzyme Specific Activity Unit Definition: One mannanase unit is defined as the amount of enzyme required to generate 1 [symbol] mole of mannose reducing sugar equivalents per minute under the conditions of the assay. FIG. 4A shows the mannanase activity displayed by the three forms of Bsp Man4 at pH 8.2. FIG. 4B shows the mannanase activity displayed by the three forms of Bsp Man4 at pH 5.0.

### Example 6

### Cleaning Performance of Bsp Man4 and Fragments Thereof

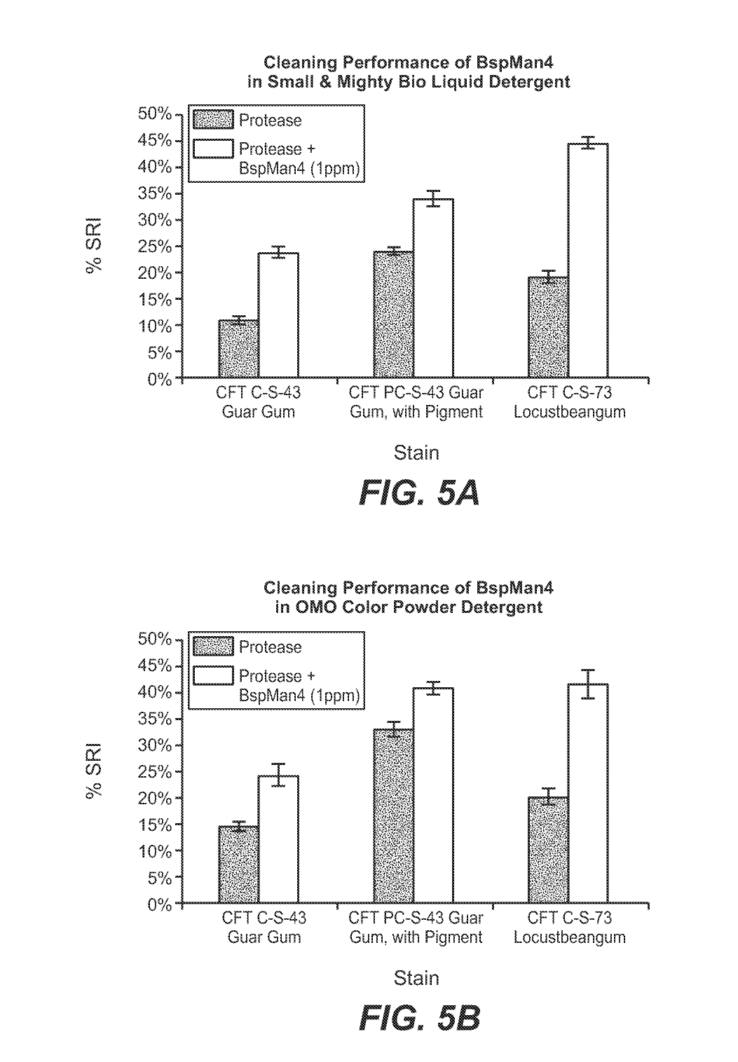

The cleaning performance of Bsp Man4 was tested in a Launder-O-meter LP-2 (Atlas Electric Devices Co., Chicago, Ill.) or equivalent using the CS-43 (Guar Gum), CS-73 (Locust Bean Gum), and PCS-43 (pigment stained Guar Gum) swatches purchased from Center for Testmaterials, The Netherlands. The cleaning performance of Bsp Man4 was tested in combination with a protease (PURAFECT® or PURAFECT® Prime). Swatches were cut to 3 cm×3 cm in size, read on a Konica Minolta CR-400 reflectometer for pre-wash RGB values, and 4 swatches of each stain type (12 g including ballast soil) were added to each test beaker along with 6 stainless steel balls. Water hardness was adjusted to a final concentration of 100 ppm and used to dilute the detergents. The commercially available detergent OMO color powder (Unilever) was heat-inactivated and used at a dose of 5.25 g/L. The commercially available Small and Mighty bio liquid detergent (Unilever) contained no enzymes and was used without heat-inactivation at a dose of 2.33 g/L. Varying doses (0.25, 1 and 2.5 ppm) of Bsp Man4 along with 0.5 ppm of PURAFECT® Prime for liquid detergent or 0.8 ppm of PURAFECT® for powder detergent were added to each beaker. The washing cycle time was 45 minutes at 40° C. After the wash, the swatches were removed, rinsed for 5 minutes in cold tap water, spun in a laundry centrifuge and laid flat in heating cabinet to dry. The dry swatches were covered with dark cloth at room temperature and stain removal was assessed by measuring the RGB values with a Konica Minolta CR-400 reflectometer. Stain removal was calculated using the RGB color values as the difference of the post- and pre-cleaning RGB color measurements for each swatch. The % SR readings for 1 ppm Bsp Man4 dose are shown in FIGS. 5A and 5B.

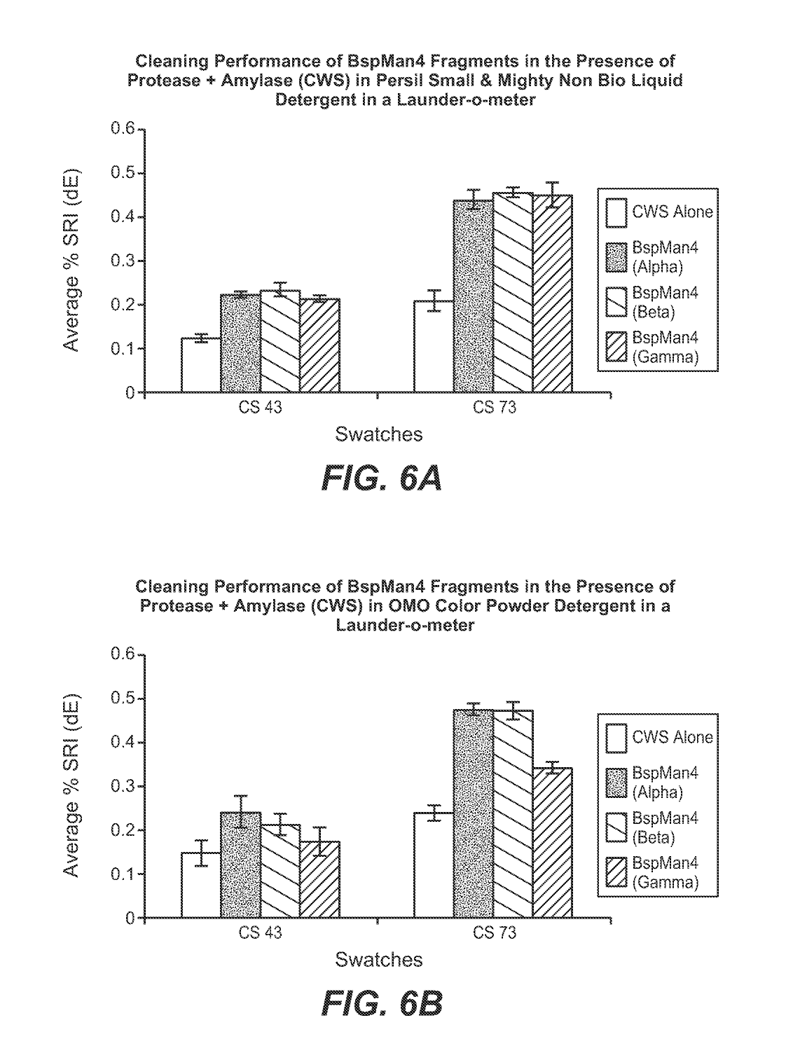

Three forms of Bsp Man4 were identified by SDS-PAGE: alpha (MW˜100 kDa); beta (MW˜70 kDa); and gamma (MW˜50 kDa). The sample containing the gamma form of Bsp Man4 was a mixture including unrelated proteins, with Bsp Man4 present at 30% w/w. The cleaning performance of the three forms of Bsp Man4 was tested in a Launder-O-meter LP-2 (Atlas Electric Devices Co., Chicago, Ill.) or equivalent using the CS-43 (Guar Gum) and CS-73 (Locust Bean Gum) swatches purchased from Center for Testmaterials, The Netherlands. The cleaning performance of the protein was tested in combination with protease (PURAFECT® or PURAFECT® Prime) plus amylase (ACE prime described in WO2010/115021 or POWERASE®). Swatches were cut to 3 cm×3 cm in size, read on a Konica Minolta CR-400 reflectometer for pre-wash RGB values, and 4 swatches of each stain type (12 g including ballast soil) were added to each test beaker along with 6 stainless steel balls. Water hardness was adjusted to a final concentration of 100 ppm. The commercially available detergent OMO color powder (Unilever) was heat-inactivated and used at a dose of 5.25 g/L diluted in 50 mM CAPS buffer pH 10.0. The commercially available Persil Small and Mighty bio liquid detergent (Unilever) contained no enzymes and was used without heat-inactivation at a dose of 2.33 g/L diluted in 50 mM HEPES buffer pH 8.2. Varying doses (0.25, 0.5, 1 and 2.5 ppm) of Bsp Man4 fragments along with 0.5 ppm PURAFECT® Prime and 0.1 ppm ACE prime with liquid detergents and 0.8 ppm PURAFECT® and 0.2 ppm POWERASE® with powder detergent were added to each beaker. The washing cycle time was 45 minutes at 40° C. After the wash, the swatches were removed, rinsed for 5 minutes in cold tap water, spun in a laundry centrifuge and laid flat in heating cabinet to dry. The dry swatches were covered with dark cloth at room temperature and stain removal was assessed by measuring the RGB values with a Konica Minolta CR-400 reflectometer. The % SR readings for 0.25 ppm Bsp Man4 fragments are shown in FIG. 6A (OMO color powder detergent) and 6B (Persil Small & Mighty liquid detergent).

### Example 7

### Comparison of Bsp Man4 to Other Mannanases

### A. Identification of Homologous Mannanases

Homologs were identified by BLAST search (Altschul et al., Nucleic Acids Res. 25:3389-402, 1997) against the NCBI non-redundant protein database (nr) using the amino acid sequence of the mature form of Bsp Man4 (SEQ ID NO:8) as the query sequence. Only sequences with a percent identity of 40% or higher were retained. Percent identity (PID) is defined as the number of identical residues divided by the number of aligned residues in the pairwise alignment. Table 7-1 provides the list of sequences identified having a percent identity of 40% or higher to Bsp Man4. Table 7-1 provides NCBI and SEQ ID NOs. for each homolog, as well as the length (number of amino acids) of each sequence; and the PID (percent identity).

### B. Alignment of Homologous Mannanase Sequences

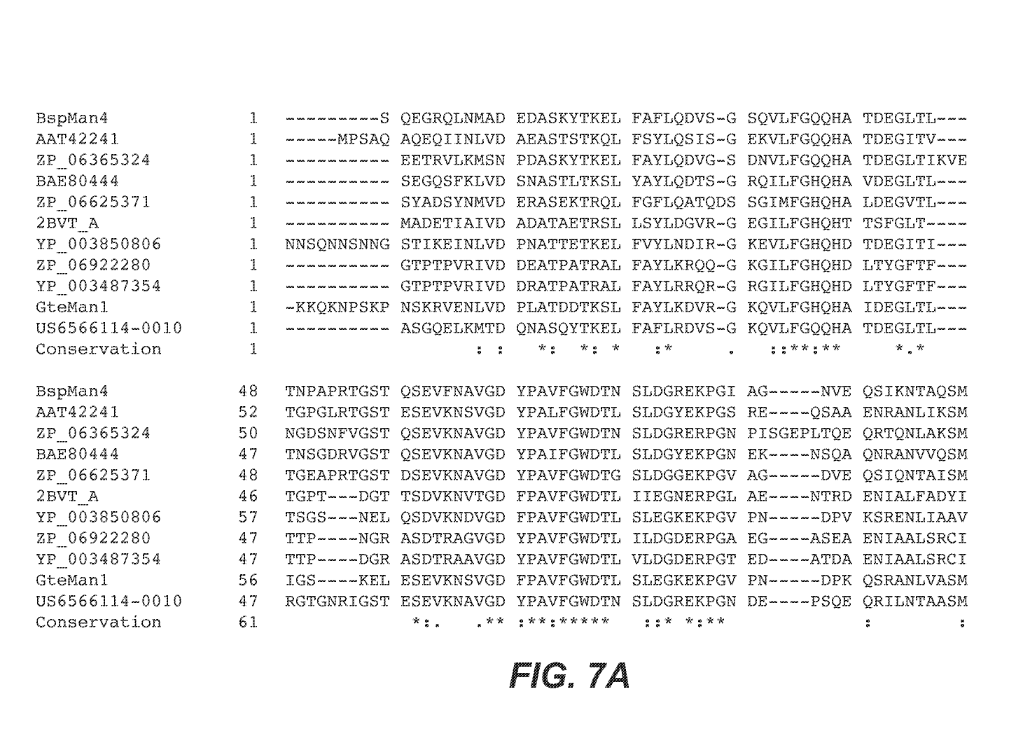

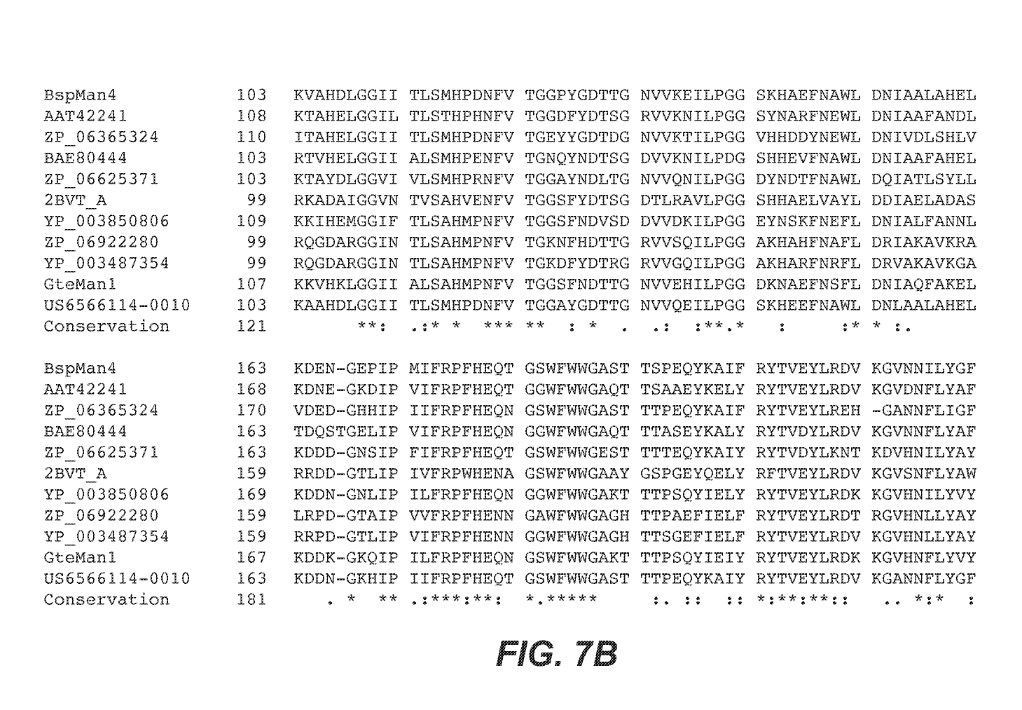

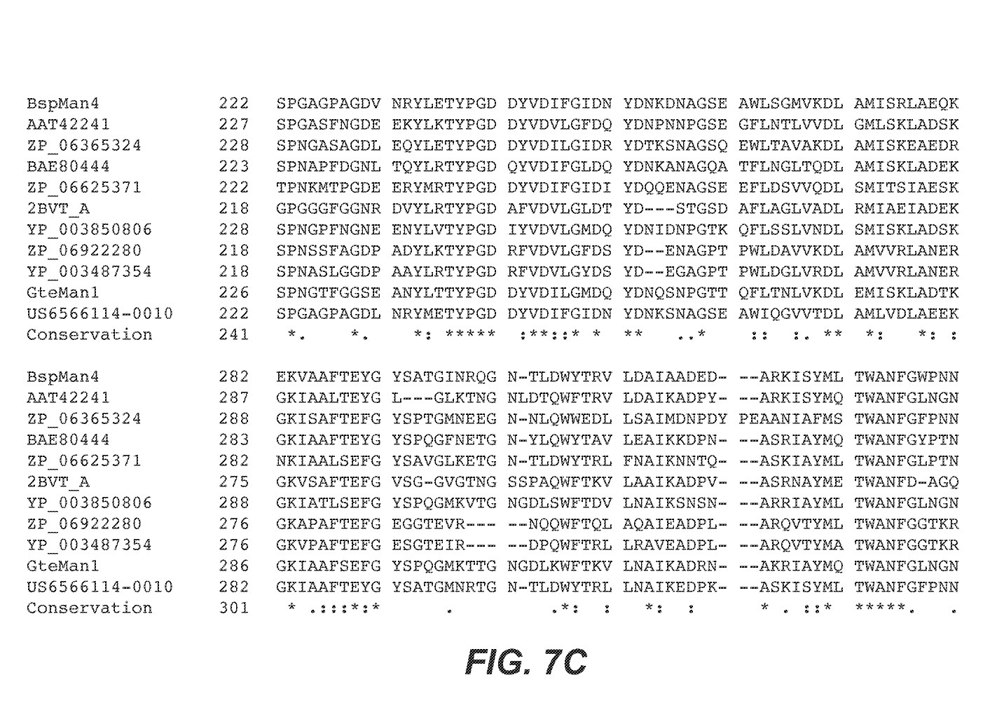

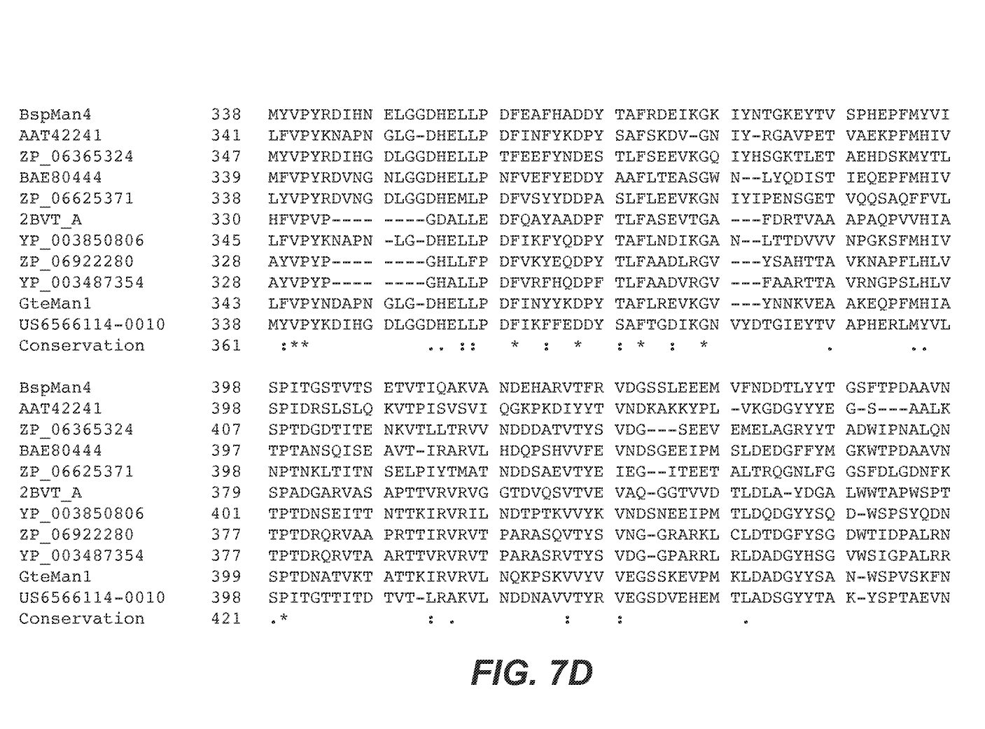

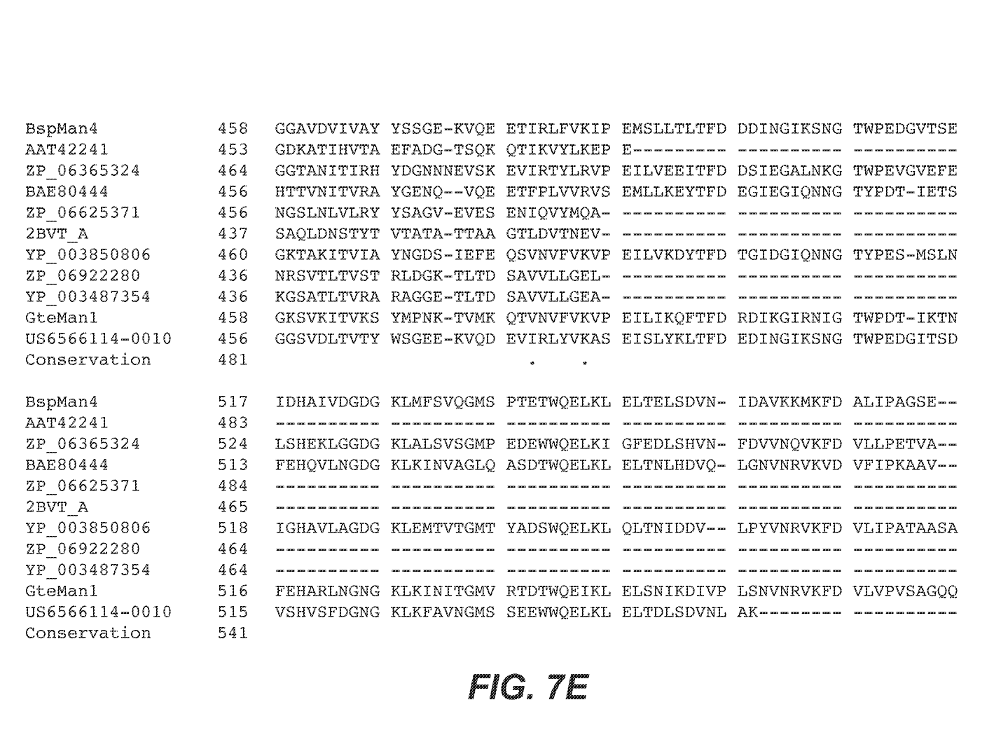

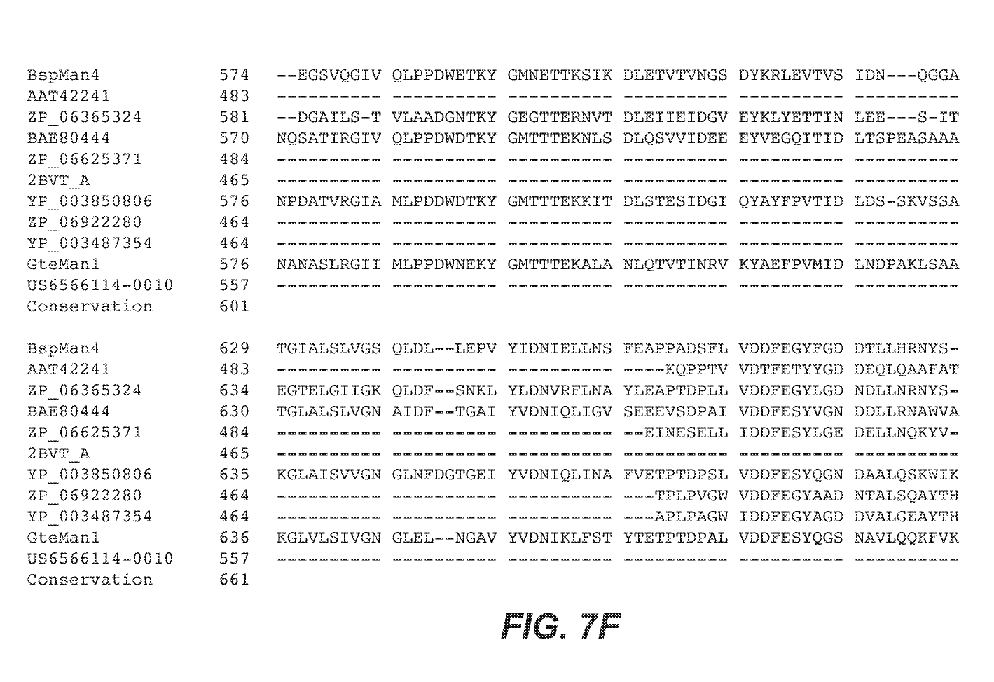

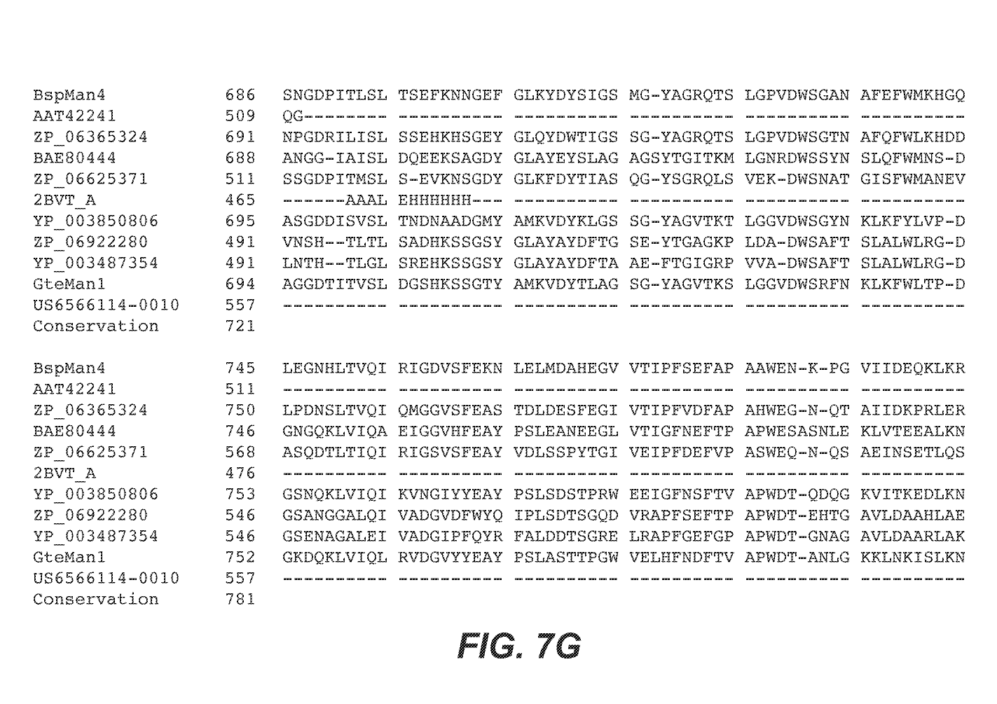

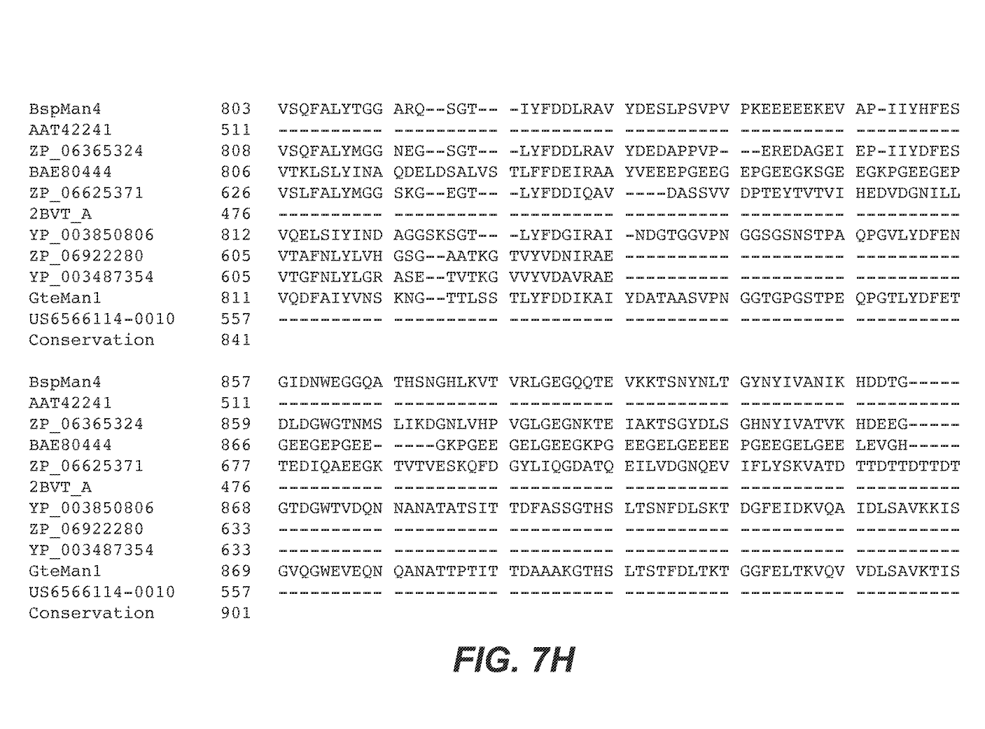

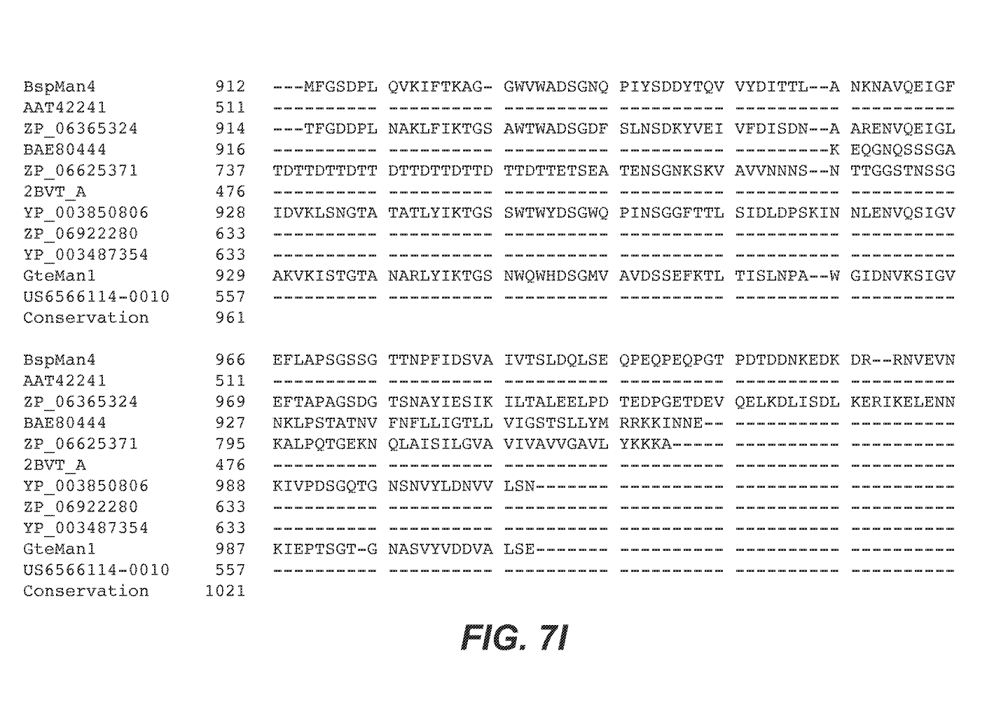

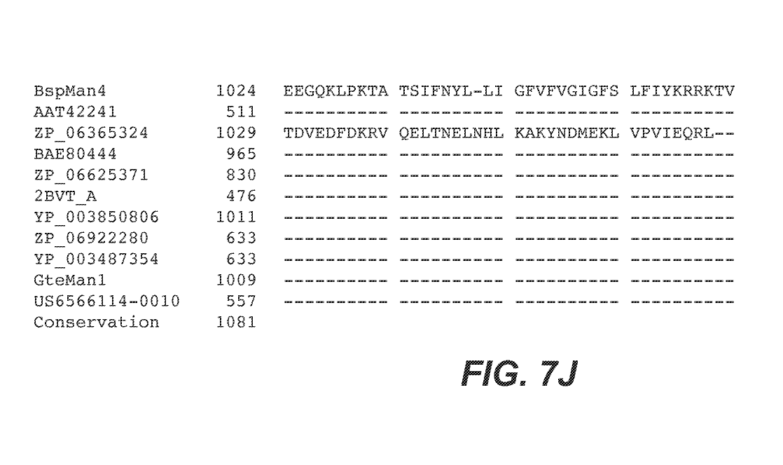

The sequences of Bsp Man4 and selected homologs were multiply aligned using CLUSTALW software (Thompson et al., Nucleic Acids Res, 22:4673-4680, 1994) using default parameters. The alignment was refined with MUSCLE (MUltiple Sequence Comparison by Log-Expectation, Edgar, Nucleic Acids Res, 32:1792-1797, 2004) using default parameters. For homologous sequences, only regions that correspond to seed sequences are shown. Redundant sequences that are 98% or higher in PID were not included in further analyses. FIG. 7 shows the alignment of Bsp Man4 with homologous mannanases.

### C. Phylogenetic Tree

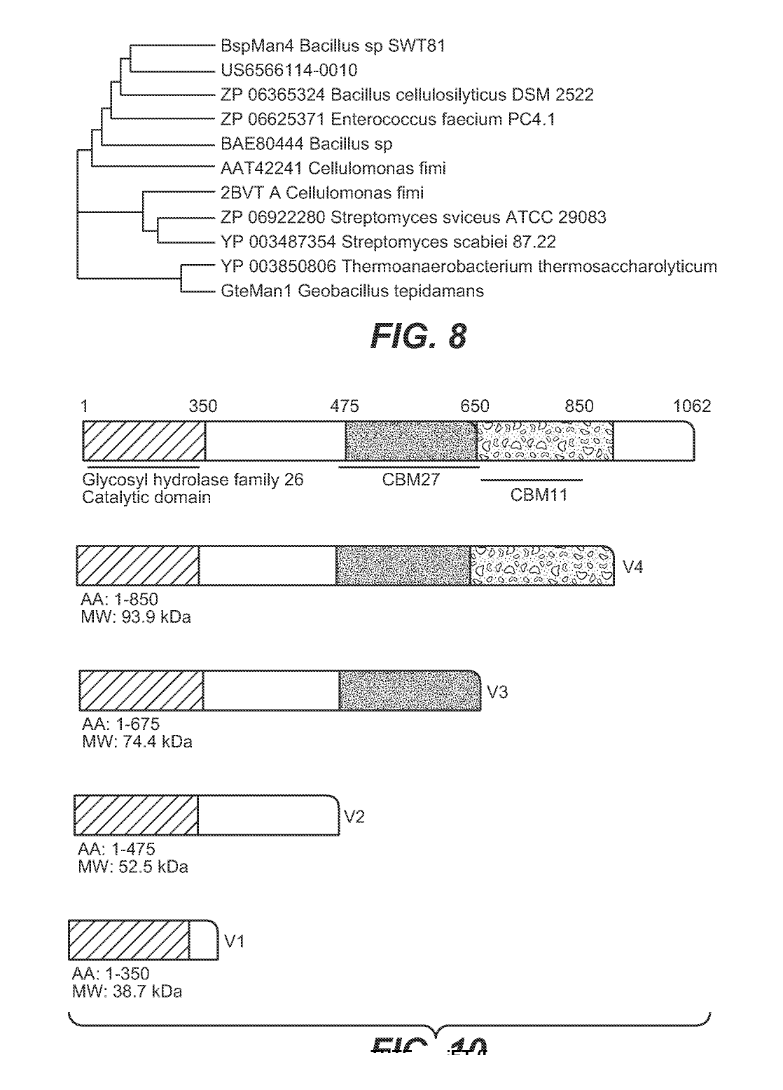

A phylogenetic tree was built for Bsp Man4 with the Neighbor-Joining algorithm using ClustalW software with 10000 bootstraps based on the refined alignments described above. Bootstrapping was used to assess the reliability of the tree branches (Felsenstein, Evolution 39:783-791, 1985). Other ClustalW parameters were set at the default values. The phylogenetic tree was rendered using the program PhyloWidget: web-based visualizations for the tree of life at www.phylowidget.org (Jordan and Piel, Bioinformatics, 24:1641-1642, 2008). The phyogenetic tree for Bsp Man4 is shown in FIG. 8.

TABLE 7-1
List of Bsp Man4 Homologs with a Percent Identity of 40 or Greater to the
Mature Form of SEQ ID NO: 8

| | SEQ ID | LENGTH | % IDENTITY |
|---|---|---|---|
| Homolog | NO: | (# residues) | (PID) |
| U.S. Pat. No. 6,566,114-0010 | 15 | 586 | 74.6 |
| ZP-06365324 | 16 | 1121 | 54.2 |
| AAT42241 | 17 | 510 | 54.0 |
| BAE80444 | 18 | 997 | 49.8 |
| ZP_06625371 | 19 | 854 | 47.1 |
| 2BVT_A | 20 | 475 | 44.0 |
| Gte Man1 | 21 | 1008 | 43.3 |
| YP_003850806 | 22 | 1410 | 43.0 |
| ZP_06922280 | 23 | 786 | 42.0 |
| YP_003487354 | 24 | 667 | 41.0 |

TABLE 7-2
List of Bsp Man4 Homologs with a Percent Identity of 40 or Greater to
the Catalytic Domain (296 residues) of SEQ ID NO: 9

| Homolog | Length | PID(%) |
|---|---|---|
| U.S. Pat. No. 6,566,114-0010 | 586 | 83.4 |
| ZP_06365324 | 1121 | 70.8 |
| BAE80444 | 997 | 67.2 |
| ZP_06625371 | 854 | 66.9 |
| AAT42241 | 510 | 64.9 |
| Gte Man1 | 294 | 61.6 |
| YP_003850806 | 1410 | 55.3 |
| ZP_06922280 | 786 | 51 |
| YP_003487354 | 667 | 51 |
| 2BVT_A | 475 | 50.2 |

### Example 8

### Prediction of Functional Domains of Bsp Man4

The location of functional domains such as the catalytic region and carbohydrate-binding domains of Bsp Man4 was determined using reference sequences within the BLAST result list using the Conserved Domain Search Service (CD Search) tool located in the NCBI web site. CD-Search uses RPS-BLAST (Reverse Position-Specific BLAST) to compare a query sequence against position-specific score matrices that have been prepared from conserved domain alignments present in the Conserved Domain Database (CDD). The results of CD-Search are presented as annotated protein domains on the user query sequence. The protein sequence of homolog D2M1G9 (TrEMBL, former NCBI ZP_{—}06365324) was entered into the CD Search tool to identify the catalytic and carbohydrate binding domains of Bsp Man4. The amino acid sequence of D2M1G9 shares 54.2% identity with Bsp Man4.

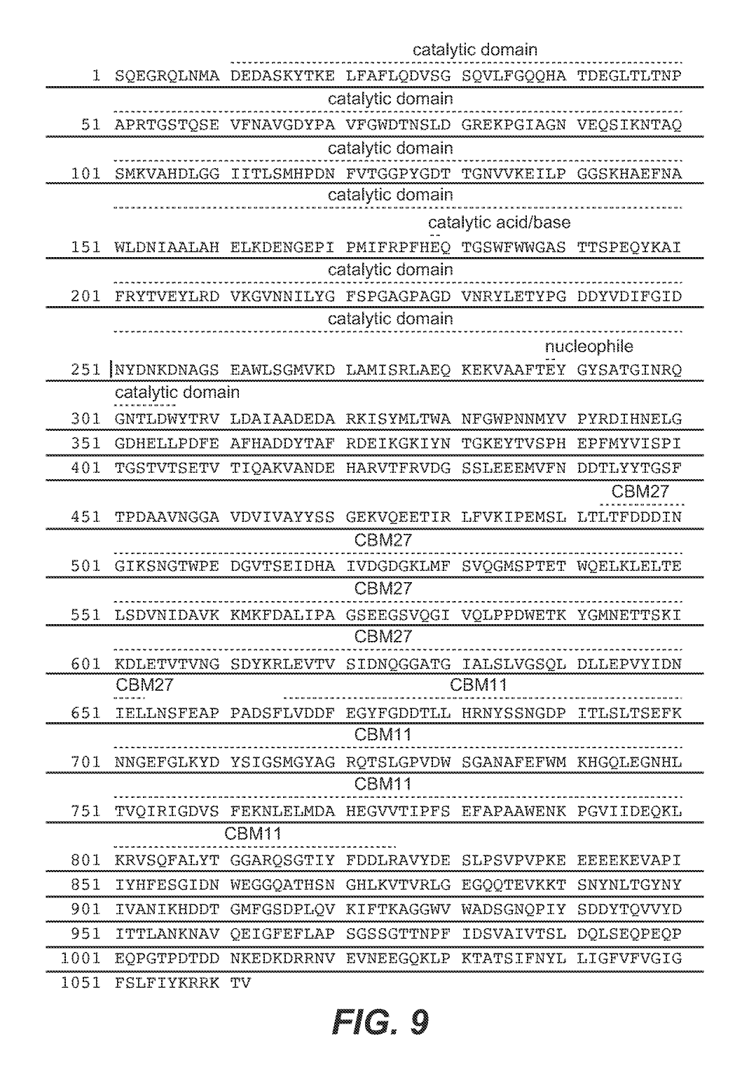

Functional domains were predicted using ClustalW alignments by AlignX within Vector NTI (Invitrogen). Based on the alignment with D2M1G9, the catalytic domain of Bsp Man4 was predicted to be 296 amino acids in length, starting at position D11 and ending with position W306. The binding module CBM27 was predicted to be 161 amino acids in length, starting at position L493 and ending with position L653. The binding module CBM11 was predicted to be 160 amino acids in length, starting at position L666 and ending with position R825. A complete description of the carbohydrate binding module family classifications can be found in the CAZy carbohydrate active enzymes database (www.cazy.org/Carbohydrate-Binding-Modules.html). Catalytic residues of Bsp Man4 were predicted to be at E179 and E289, using a literature reference describing the structure of Cellulomonas fimi CfMan26A (Le Nours et al., Biochemistry 44:12700-8, 2005). All positions were calculated from the start of the mature protein sequence. FIG. 9 shows the functional domains of BspMan 4.

The amino acid sequence of the catalytic domain of Bsp Man4 is set forth as SEQ ID NO:9:

DEDASKYTKELFAFLQDVSGSQVLFGQQHATDEGLTLTNPAPRTGSTQSE
VFNAVGDYPAVFGWDTNSLDGREKPGIAGNVEQSIKNTAQSMKVAHDLGG
IITLSMHPDNFVTGGPYGDTTGNVVKEILPGGSKHAEFNAWLDNIAALAH
ELKDENGEPIPMIFRPFHEQTGSWFWWGASTTSPEQYKAIFRYTVEYLRD
VKGVNNILYGFSPGAGPAGDVNRYLETYPGDDYVDIFGIDNYDNKDNAGS
EAWLSGMVKDLAMISRLAEQKEKVAAFTEYGYSATGINRQGNTLDW.

Next, a homology model of Bsp Man4 was built by threading the amino acid sequence of Bsp Man4 onto to the three dimensional structure of a Cellulomonas fimi mannanase. The following steps to construct the homology model were accomplished using the program suite “MOE” provided by Chemical Computing Group Inc., (Montreal, Quebec, Canada). The first step involved using the protein sequence of Bsp Man4 to search for homologous sequences of known structures in the Protein Data Bank (www.rcsb.org/pdb/home/home.do). From this search, the Cellulomonas fimi mannanase (pdb entry 2×2Y) was identified, and the shared identity between 2×2Y and Bsp Man4 was found to be 40.4%. The next step involved threading the sequence of Bsp Man4onto related elements of the known sequence of the Cellulomonas fimi mannanase. The threading process itself includes several constraints. One such constraint involves keeping the main chain and side chain structure of the conserved residues the same. Another constraint involves keeping the main chain atoms fixed, while searching for rotamers of the replaced side chains of non conserved residues which are most compatible with the ensemble of neighboring atoms within the model. When residues were inserted, a loop structure library was used to model the possible insertions. The entire threading process was repeated 10 times with the potential for selecting different rotamers. All models were subjected to limited energy minimization, followed by selection of the model having the lowest energy. Amino acid sequences of truncated species of Bsp Man4, based on the homology model are shown below.

The amino acid sequence of truncated species 1 of Bsp Man4 is set forth as SEQ ID NO:10.

RQLNMADEDASKYTKELFAFLQDVSGSQVLFGQQHATDEGLTLTNPAPRT
GSTQSEVFNAVGDYPAVFGWDTNSLDGREKPGIAGNVEQSIKNTAQSMKV
AHDLGGIITLSMHPDNFVTGGPYGDTTGNVVKEILPGGSKHAEFNAWLDN
IAALAHELKDENGEPIPMIFRPFHEQTGSWFWWGASTTSPEQYKAIFRYT
VEYLRDVKGVNNILYGFSPGAGPAGDVNRYLETYPGDDYVDIFGIDNYDN
KDNAGSEAWLSGMVKDLAMISRLAEQKEKVAAFTEYGYSATGINRQGNTL
DWYTRVLDAIAADEDARKISYMLTWANFGWPNNMYVPYRDIHNELGGDHE
LLPDFEAFHADDYTAFRDEIKGKIYNTGKEYTVSPHEPFMYVISPITGST
VTSETVTIQAKVANDEHARVTFRVDGSSLEEEMVFNDDTLYYTGSFTPDA
AVNGGAVDVIVAYYSSGEK.

The amino acid sequence of truncated species 2 of Bsp Man4 is set forth as SEQ ID NO:11.

RQLNMADEDASKYTKELFAFLQDVSGSQVLFGQQHATDEGLTLTNPAPRT
GSTQSEVFNAVGDYPAVFGWDTNSLDGREKPGIAGNVEQSIKNTAQSMKV
AHDLGGIITLSMHPDNFVTGGPYGDTTGNVVKEILPGGSKHAEFNAWLDN
IAALAHELKDENGEPIPMIFRPFHEQTGSWFWWGASTTSPEQYKAIFRYT
VEYLRDVKGVNNILYGFSPGAGPAGDVNRYLETYPGDDYVDIFGIDNYDN
KDNAGSEAWLSGMVKDLAMISRLAEQKEKVAAFTEYGYSATGINRQGNTL
DWYTRVLDAIAADEDARKISYMLTWANFGWPNNMYVPYRDIHNELGGDHE
LLPDFEAFHADDYTAFRDEIKGKIYNTGKEYTV.

In addition, the amino acid sequence of the alpha, beta and gamma forms of recombinant Bsp Man4 were determined by Edman degradation and mass spectroscopy.

The alpha form of Bsp Man4 comprises residues 1-849 of SEQ ID NO:7. The amino acid sequence of the alpha form of Bsp Man4 (MW˜100 kDa by SDS-PAGE, or MW˜94 kDa by mass spectroscopy) is set forth as SEQ ID NO:12.

AGKSQEGRQLNMADEDASKYTKELFAFLQDVSGSQVLFGQQHATDEGLTL
TNPAPRTGSTQSEVFNAVGDYPAVFGWDTNSLDGREKPGIAGNVEQSIKN
TAQSMKVAHDLGGIITLSMHPDNFVTGGPYGDTTGNVVKEILPGGSKHAE
FNAWLDNIAALAHELKDENGEPIPMIFRPFHEQTGSWFWWGASTTSPEQY
KAIFRYTVEYLRDVKGVNNILYGFSPGAGPAGDVNRYLETYPGDDYVDIF
GIDNYDNKDNAGSEAWLSGMVKDLAMISRLAEQKEKVAAFTEYGYSATGI
NRQGNTLDWYTRVLDAIAADEDARKISYMLTWANFGWPNNMYVPYRDIHN
ELGGDHELLPDFEAFHADDYTAFRDEIKGKIYNTGKEYTVSPHEPFMYVI
SPITGSTVTSETVTIQAKVANDEHARVTFRVDGSSLEEEMVFNDDTLYYT
GSFTPDAAVNGGAVDVIVAYYSSGEKVQEETIRLFVKIPEMSLLTLTFDD
DINGIKSNGTWPEDGVTSEIDHAIVDGDGKLMFSVQGMSPTETWQELKLE
LTELSDVNIDAVKKMKFDALIPAGSEEGSVQGIVQLPPDWETKYGMNETT
KSIKDLETVTVNGSDYKRLEVTVSIDNQGGATGIALSLVGSQLDLLEPVY
IDNIELLNSFEAPPADSFLVDDFEGYFGDDTLLHRNYSSNGDPITLSLTS
EFKNNGEFGLKYDYSIGSMGYAGRQTSLGPVDWSGANAFEFWMKHGQLEG
NHLTVQIRIGDVSFEKNLELMDAHEGVVTIPFSEFAPAAWENKPGVIIDE
QKLKRVSQFALYTGGARQSGTIYFDDLRAVYDESLPSVPVPKEEEEEKE.

The beta form of Bsp Man4 comprises residues 1-669 of SEQ ID NO:7. The amino acid sequence of the beta form of Bsp Man4 (MW˜70 kDa by SDS-PAGE, or MW˜74 kDa by mass spectroscopy) is set forth as SEQ ID NO:13.

AGKSQEGRQLNMADEDASKYTKELFAFLQDVSGSQVLFGQQHATDEGLTL
TNPAPRTGSTQSEVFNAVGDYPAVFGWDTNSLDGREKPGIAGNVEQSIKN
TAQSMKVAHDLGGIITLSMHPDNFVTGGPYGDTTGNVVKEILPGGSKHAE
FNAWLDNIAALAHELKDENGEPIPMIFRPFHEQTGSWFWWGASTTSPEQY
KAIFRYTVEYLRDVKGVNNILYGFSPGAGPAGDVNRYLETYPGDDYVDIF
GIDNYDNKDNAGSEAWLSGMVKDLAMISRLAEQKEKVAAFTEYGYSATGI
NRQGNTLDWYTRVLDAIAADEDARKISYMLTWANFGWPNNMYVPYRDIHN
ELGGDHELLPDFEAFHADDYTAFRDEIKGKIYNTGKEYTVSPHEPFMYVI
SPITGSTVTSETVTIQAKVANDEHARVTFRVDGSSLEEEMVFNDDTLYYT
GSFTPDAAVNGGAVDVIVAYYSSGEKVQEETIRLFVKIPEMSLLTLTFDD
DINGIKSNGTWPEDGVTSEIDHAIVDGDGKLMFSVQGMSPTETWQELKLE
LTELSDVNIDAVKKMKFDALIPAGSEEGSVQGIVQLPPDWETKYGMNETT
KSIKDLETVTVNGSDYKRLEVTVSIDNQGGATGIALSLVGSQLDLLEPVY
IDNIELLNSFEAPPADSFL.

The gamma form of Bsp Man4 comprises residues 1-494 of SEQ ID NO:7. The amino acid sequence of the gamma form of Bsp Man4 (MW˜50 kDa by SDS-PAGE, or MW˜54 kDa by mass spectroscopy) is set forth as SEQ ID NO:14.

AGKSQEGRQLNMADEDASKYTKELFAFLQDVSGSQVLFGQQHATDEGLTL
TNPAPRTGSTQSEVFNAVGDYPAVFGWDTNSLDGREKPGIAGNVEQSIKN
TAQSMKVAHDLGGIITLSMHPDNFVTGGPYGDTTGNVVKEILPGGSKHAE
FNAWLDNIAALAHELKDENGEPIPMIFRPFHEQTGSWFWWGASTTSPEQY
KAIFRYTVEYLRDVKGVNNILYGFSPGAGPAGDVNRYLETYPGDDYVDIF
GIDNYDNKDNAGSEAWLSGMVKDLAMISRLAEQKEKVAAFTEYGYSATGI
NRQGNTLDWYTRVLDAIAADEDARKISYMLTWANFGWPNNMYVPYRDIHN
ELGGDHELLPDFEAFHADDYTAFRDEIKGKIYNTGKEYTVSPHEPFMYVI
SPITGSTVTSETVTIQAKVANDEHARVTFRVDGSSLEEEMVFNDDTLYYT
GSFTPDAAVNGGAVDVIVAYYSSGEKVQEETIRLFVKIPEMS.

In further embodiments, additional truncated forms of Bsp Man4 are provided. One form comprises residues 1 to 350 of SEQ ID NO:8, another form comprises residues 1 to 475 of SEQ ID NO:8, another form comprises residues 1 to 675 of SEQ ID NO:8, and yet another form comprises residues 1 to 850 of SEQ ID NO:8 (as described below).

### Example 9

### Cloning of the Bacillus sp. Mannanase Bsp Man4 Variants

Different length of Bacillus sp. mannanase Bsp Man4 variants were obtained by PCR from Bacillus sp. mannanase Bsp Man4 wild type plasmid DNA pZQ186 (aprE-Bsp Man4). Primers were designed based on Bacillus sp. mannanase Bsp Man4 full-length gene sequences and Bsp Man4 Pfam domain structures (The Pfam protein families database: M. Punta, P. C. Coggill, R. Y. Eberhardt, J. Mistry, J. Tate, C. Boursnell, N. Pang, K. Forslund, G. Ceric, J. Clements, A. Heger, L. Holm, E. L. L. Sonnhammer, S. R. Eddy, A. Bateman, R. D. Finn, Nucleic Acids Research (2012) Database Issue 40:D290-D301). The diagrams of the truncations can be found in FIG. 10. Primers used in this study are: For: 5′-ACTAGCCGACTAGTTCACAAGAAGGGCGTCAACTTAAC-3′ (SEQ ID NO:25), v1_Rev: 5′-CTTACGGG CTCGAGTTAACCTAATTCATTGTGGATATCACG-3′ (SEQ ID NO:26, v2_Rev: 5′-CTTACGGG CTCGAGTTATTGGACTTTTTCTCCACTAGAATAATAAG-3′ (SEQ ID NO:27, v3_Rev: 5′-CTTACGGG CTCGAGTTACCCAAAATAACCTTCAAAATCATC-3′ (SEQ ID NO:28, v4 Rev: 5′-CTTACGGGCTCGAGTTAAATAGGAGCGACCTCTTTTTCCTCTTC-3′ (SEQ ID NO:29). The PCR primers contain Spe I restriction enzyme sites and Xho I restriction enzyme sites for cloning purpose. PCR was performed using a thermocycler with KOD-plus polymerase (TOYOBA) according to the instructions of the manufacturer (annealing temperature of 58° C.). The nucleic acid sequences of PCR products are confirmed by sequencing analysis.

The PCR products were digested with Spe I and Xho I (New England Biolabs) and then ligated into expression vector p2JM. The ligation mixture was transformed into E. coli TOP10 chemical competent cells following manufacture's protocol (Life Technology). Transformed cells were then plated on Luria Broth agar plates and selected by 50 ppm ampicillin antibiotics, incubated at 37 degree over night. Positive clones containing the correct inserts were confirmed by sequencing analysis.

The nucleotide sequence of the Bacillus sp. mannanase Bsp Man4v1 gene is set forth as SEQ ID NO: 30.

(SEQ ID NO: 30)
TCACAAGAAGGGCGTCAACTTAACATGGCAGATGAGGATGCTTCAAAGTA
TACGAAGGAGTTATTTGCTTTTCTTCAAGATGTAAGTGGTTCACAAGTGT
TATTTGGACAACAGCATGCAACAGATGAAGGATTAACTTTAACAAATCCA
GCTCCAAGAACAGGTTCCACTCAATCTGAAGTTTTCAATGCAGTTGGGGA
TTATCCAGCTGTGTTTGGATGGGACACGAATAGCCTAGATGGTCGTGAAA
AGCCTGGCATTGCAGGTAATGTAGAACAAAGTATAAAAAATACGGCTCAG
TCCATGAAAGTGGCTCATGATTTAGGAGGGATTATTACACTAAGCATGCA
CCCAGATAATTTTGTAACAGGGGGTCCTTATGGTGATACAACAGGGAATG
TTGTAAAAGAAATTCTTCCAGGTGGATCAAAACATGCAGAGTTTAACGCG
TGGTTGGACAATATTGCTGCGCTTGCTCACGAGCTGAAAGATGAGAATGG
TGAACCTATTCCGATGATTTTTCGGCCATTCCATGAACAAACAGGATCTT
GGTTTTGGTGGGGAGCAAGCACAACTTCACCCGAACAATATAAAGCGATT
TTTCGTTATACAGTAGAATATTTGCGAGATGTTAAAGGCGTAAATAATAT
TTTATATGGCTTTTCACCTGGGGCGGGACCTGCTGGAGATGTAAATCGCT
ATTTAGAAACATATCCAGGGGATGATTACGTTGATATTTTCGGTATTGAC
AATTATGACAATAAAGACAATGCAGGGTCAGAAGCTTGGTTAAGTGGTAT
GGTCAAAGACTTGGCGATGATTAGCCGATTAGCTGAACAAAAAGAAAAAG
TAGCGGCTTTTACTGAGTATGGGTACAGTGCAACCGGAATTAATCGTCAA
GGGAATACATTAGACTGGTACACACGTGTATTAGATGCGATTGCTGCTGA
TGAAGACGCACGTAAAATATCATACATGTTGACATGGGCGAACTTTGGTT
GGCCGAATAATATGTATGTTCCTTATCGTGATATCCACAATGAATTAGGT
TAA

The amino acid sequence of the Bacillus sp. mannanase Bsp Man4v1 protein is set forth as SEQ ID NO: 31. The signal peptide is shown in italics and lowercase. There is a restriction enzyme site introduced between signal peptide and first codon of Bacillus sp. mannanase Bsp Man4v1, which is shown in lowercase and underline.

(SEQ ID NO: 31)
mrskklwisllfaltliftmafsnmsaqatsSQEGRQLNMADEDASKYTK
ELFAFLQDVSGSQVLFGQQHATDEGLTLTNPAPRTGSTQSEVFNAVGDYP
AVFGWDTNSLDGREKPGIAGNVEQSIKNTAQSMKVAHDLGGIITLSMHPD
NFVTGGPYGDTTGNVVKEILPGGSKHAEFNAWLDNIAALAHELKDENGEP
IPMIFRPFHEQTGSWFWWGASTTSPEQYKAIFRYTVEYLRDVKGVNNILY
GFSPGAGPAGDVNRYLETYPGDDYVDIFGIDNYDNKDNAGSEAWLSGMVK
DLAMISRLAEQKEKVAAFTEYGYSATGINRQGNTLDWYTRVLDAIAADED
ARKISYMLTWANFGWPNNMYVPYRDIHNELG

The amino acid sequence of the mature form of Bsp Man4v1 is set forth as SEQ ID NO: 32.

(SEQ ID NO: 32)
SQEGRQLNMADEDASKYTKELFAFLQDVSGSQVLFGQQHATDEGLTLTNP
APRTGSTQSEVFNAVGDYPAVFGWDTNSLDGREKPGIAGNVEQSIKNTAQ
SMKVAHDLGGIITLSMHPDNFVTGGPYGDTTGNVVKEILPGGSKHAEFNA
WLDNIAALAHELKDENGEPIPMIFRPFHEQTGSWFWWGASTTSPEQYKAI
FRYTVEYLRDVKGVNNILYGFSPGAGPAGDVNRYLETYPGDDYVDIFGID
NYDNKDNAGSEAWLSGMVKDLAMISRLAEQKEKVAAFTEYGYSATGINRQ
GNTLDWYTRVLDAIAADEDARKISYMLTWANFGWPNNMYVPYRDIHNELG

The nucleotide sequence of the Bacillus sp. mannanase Bsp Man4v2 gene is set forth as SEQ ID NO: 33.

(SEQ ID NO: 33)
TCACAAGAAGGGCGTCAACTTAACATGGCAGATGAGGATGCTTCAAAGTA
TACGAAGGAGTTATTTGCTTTTCTTCAAGATGTAAGTGGTTCACAAGTGT
TATTTGGACAACAGCATGCAACAGATGAAGGATTAACTTTAACAAATCCA
GCTCCAAGAACAGGTTCCACTCAATCTGAAGTTTTCAATGCAGTTGGGGA
TTATCCAGCTGTGTTTGGATGGGACACGAATAGCCTAGATGGTCGTGAAA
AGCCTGGCATTGCAGGTAATGTAGAACAAAGTATAAAAAATACGGCTCAG
TCCATGAAAGTGGCTCATGATTTAGGAGGGATTATTACACTAAGCATGCA
CCCAGATAATTTTGTAACAGGGGGTCCTTATGGTGATACAACAGGGAATG
TTGTAAAAGAAATTCTTCCAGGTGGATCAAAACATGCAGAGTTTAACGCG
TGGTTGGACAATATTGCTGCGCTTGCTCACGAGCTGAAAGATGAGAATGG
TGAACCTATTCCGATGATTTTTCGGCCATTCCATGAACAAACAGGATCTT
GGTTTTGGTGGGGAGCAAGCACAACTTCACCCGAACAATATAAAGCGATT
TTTCGTTATACAGTAGAATATTTGCGAGATGTTAAAGGCGTAAATAATAT
TTTATATGGCTTTTCACCTGGGGCGGGACCTGCTGGAGATGTAAATCGCT
ATTTAGAAACATATCCAGGGGATGATTACGTTGATATTTTCGGTATTGAC
AATTATGACAATAAAGACAATGCAGGGTCAGAAGCTTGGTTAAGTGGTAT
GGTCAAAGACTTGGCGATGATTAGCCGATTAGCTGAACAAAAAGAAAAAG
TAGCGGCTTTTACTGAGTATGGGTACAGTGCAACCGGAATTAATCGTCAA
GGGAATACATTAGACTGGTACACACGTGTATTAGATGCGATTGCTGCTGA
TGAAGACGCACGTAAAATATCATACATGTTGACATGGGCGAACTTTGGTT
GGCCGAATAATATGTATGTTCCTTATCGTGATATCCACAATGAATTAGGT
GGAGACCATGAGTTATTACCGGACTTTGAAGCTTTCCATGCGGATGACTA
CACAGCATTTCGAGATGAGATAAAAGGAAAGATATATAATACTGGAAAGG
AATATACCGTTTCTCCTCATGAGCCGTTTATGTATGTTATATCTCCGATT
ACAGGTTCTACAGTGACAAGCGAAACGGTAACAATCCAAGCAAAAGTAGC
GAATGACGAACACGCAAGAGTCACTTTCAGGGTCGATGGTTCTAGTTTGG
AAGAAGAAATGGTTTTCAATGATGACACTTTATATTATACAGGTTCTTTT
ACACCAGATGCAGCAGTGAATGGCGGAGCTGTTGATGTGATTGTAGCTTA
TTATTCTAGTGGAGAAAAAGTCCAATAA

The amino acid sequence of the Bacillus sp. mannanase Bsp Man4v2 protein is set forth as SEQ ID NO: 34. The signal peptide is shown in italics and lowercase. There is a restriction enzyme site introduced between signal peptide and first codon of Bacillus sp. mannanase Bsp Man4v2, which is shown in lowercase and underline.

(SEQ ID NO: 34)
mrskklwisllfaltliftmafsnmsaqatsSQEGRQLNMADEDASKYTK
ELFAFLQDVSGSQVLFGQQHATDEGLTLTNPAPRTGSTQSEVFNAVGDYP
AVFGWDTNSLDGREKPGIAGNVEQSIKNTAQSMKVAHDLGGIITLSMHPD
NFVTGGPYGDTTGNVVKEILPGGSKHAEFNAWLDNIAALAHELKDENGEP
IPMIFRPFHEQTGSWFWWGASTTSPEQYKAIFRYTVEYLRDVKGVNNILY
GFSPGAGPAGDVNRYLETYPGDDYVDIFGIDNYDNKDNAGSEAWLSGMVK
DLAMISRLAEQKEKVAAFTEYGYSATGINRQGNTLDWYTRVLDAIAADED
ARKISYMLTWANFGWPNNMYVPYRDIHNELGGDHELLPDFEAFHADDYTA
FRDEIKGKIYNTGKEYTVSPHEPFMYVISPITGSTVTSETVTIQAKVAND
EHARVTFRVDGSSLEEEMVFNDDTLYYTGSFTPDAAVNGGAVDVIVAYYS
SGEKVQ

The amino acid sequence of the mature form of Bsp Man4v2 is set forth as SEQ ID NO:35.

(SEQ ID NO: 35)
SQEGRQLNMADEDASKYTKELFAFLQDVSGSQVLFGQQHATDEGLTLTNP
APRTGSTQSEVFNAVGDYPAVFGWDTNSLDGREKPGIAGNVEQSIKNTAQ
SMKVAHDLGGIITLSMHPDNFVTGGPYGDTTGNVVKEILPGGSKHAEFNA
WLDNIAALAHELKDENGEPIPMIFRPFHEQTGSWFWWGASTTSPEQYKAI
FRYTVEYLRDVKGVNNILYGFSPGAGPAGDVNRYLETYPGDDYVDIFGID
NYDNKDNAGSEAWLSGMVKDLAMISRLAEQKEKVAAFTEYGYSATGINRQ
GNTLDWYTRVLDAIAADEDARKISYMLTWANFGWPNNMYVPYRDIHNELG
GDHELLPDFEAFHADDYTAFRDEIKGKIYNTGKEYTVSPHEPFMYVISPI
TGSTVTSETVTIQAKVANDEHARVTFRVDGSSLEEEMVFNDDTLYYTGSF
TPDAAVNGGAVDVIVAYYSSGEKVQ

The nucleotide sequence of the Bacillus sp. mannanase Bsp Man4v3 gene is set forth as SEQ ID NO: 36.

(SEQ ID NO: 36)
TCACAAGAAGGGCGTCAACTTAACATGGCAGATGAGGATGCTTCAAAGTA
TACGAAGGAGTTATTTGCTTTTCTTCAAGATGTAAGTGGTTCACAAGTGT
TATTTGGACAACAGCATGCAACAGATGAAGGATTAACTTTAACAAATCCA
GCTCCAAGAACAGGTTCCACTCAATCTGAAGTTTTCAATGCAGTTGGGGA
TTATCCAGCTGTGTTTGGATGGGACACGAATAGCCTAGATGGTCGTGAAA
AGCCTGGCATTGCAGGTAATGTAGAACAAAGTATAAAAAATACGGCTCAG
TCCATGAAAGTGGCTCATGATTTAGGAGGGATTATTACACTAAGCATGCA
CCCAGATAATTTTGTAACAGGGGGTCCTTATGGTGATACAACAGGGAATG
TTGTAAAAGAAATTCTTCCAGGTGGATCAAAACATGCAGAGTTTAACGCG
TGGTTGGACAATATTGCTGCGCTTGCTCACGAGCTGAAAGATGAGAATGG
TGAACCTATTCCGATGATTTTTCGGCCATTCCATGAACAAACAGGATCTT
GGTTTTGGTGGGGAGCAAGCACAACTTCACCCGAACAATATAAAGCGATT
TTTCGTTATACAGTAGAATATTTGCGAGATGTTAAAGGCGTAAATAATAT
TTTATATGGCTTTTCACCTGGGGCGGGACCTGCTGGAGATGTAAATCGCT
ATTTAGAAACATATCCAGGGGATGATTACGTTGATATTTTCGGTATTGAC
AATTATGACAATAAAGACAATGCAGGGTCAGAAGCTTGGTTAAGTGGTAT
GGTCAAAGACTTGGCGATGATTAGCCGATTAGCTGAACAAAAAGAAAAAG
TAGCGGCTTTTACTGAGTATGGGTACAGTGCAACCGGAATTAATCGTCAA
GGGAATACATTAGACTGGTACACACGTGTATTAGATGCGATTGCTGCTGA
TGAAGACGCACGTAAAATATCATACATGTTGACATGGGCGAACTTTGGTT
GGCCGAATAATATGTATGTTCCTTATCGTGATATCCACAATGAATTAGGT
GGAGACCATGAGTTATTACCGGACTTTGAAGCTTTCCATGCGGATGACTA
CACAGCATTTCGAGATGAGATAAAAGGAAAGATATATAATACTGGAAAGG
AATATACCGTTTCTCCTCATGAGCCGTTTATGTATGTTATATCTCCGATT
ACAGGTTCTACAGTGACAAGCGAAACGGTAACAATCCAAGCAAAAGTAGC
GAATGACGAACACGCAAGAGTCACTTTCAGGGTCGATGGTTCTAGTTTGG
AAGAAGAAATGGTTTTCAATGATGACACTTTATATTATACAGGTTCTTTT
ACACCAGATGCAGCAGTGAATGGCGGAGCTGTTGATGTGATTGTAGCTTA
TTATTCTAGTGGAGAAAAAGTCCAAGAAGAAACAATTCGTTTATTTGTAA
AAATTCCTGAAATGTCTTTGTTAACATTAACGTTTGATGATGATATAAAC
GGAATCAAAAGCAATGGAACATGGCCTGAAGATGGTGTAACATCTGAAAT
TGACCACGCTATTGTAGATGGAGACGGCAAGTTGATGTTCTCTGTTCAAG
GAATGTCACCTACTGAAACATGGCAAGAGCTCAAGTTAGAATTAACAGAA
CTATCAGATGTGAACATTGATGCGGTTAAGAAAATGAAGTTTGACGCGCT
TATCCCAGCAGGTAGTGAAGAAGGTTCAGTCCAAGGAATCGTACAACTTC
CACCGGATTGGGAGACGAAATATGGGATGAATGAAACAACGAAGTCAATA
AAAGACTTAGAGACTGTTACTGTTAATGGAAGCGATTATAAACGGTTGGA
AGTGACTGTTTCTATCGACAATCAAGGAGGAGCTACAGGAATCGCTTTAT
CATTAGTAGGATCCCAACTCGATTTGTTAGAACCTGTCTACATCGATAAT
ATTGAACTTCTAAATTCCTTTGAAGCACCACCAGCAGATTCTTTTCTTGT
TGATGATTTTGAAGGTTATTTTGGGTAA

The amino acid sequence of the Bacillus sp. mannanase Bsp Man4v3 protein is set forth as SEQ ID NO: 37. The signal peptide is shown in italics and lowercase. There is a restriction enzyme site introduced between signal peptide and first codon of Bacillus sp. mannanase Bsp Man4v3, which is shown in lowercase and underline.

(SEQ ID NO: 37)
MrskklwisllfaltliftmafsnmsaqatsSQEGRQLNMADEDASKYTKELFAFLQDVSGSQVLFGQ
QHATDEGLTLTNPAPRTGSTQSEVFNAVGDYPAVFGWDTNSLDGREKPGIAGNVEQ
SIKNTAQSMKVAHDLGGIITLSMHPDNFVTGGPYGDTTGNVVKEILPGGSKHAEFNA
WLDNIAALAHELKDENGEPIPMIFRPFHEQTGSWFWWGASTTSPEQYKAIFRYTVEY
LRDVKGVNNILYGFSPGAGPAGDVNRYLETYPGDDYVDIFGIDNYDNKDNAGSEAW
LSGMVKDLAMISRLAEQKEKVAAFTEYGYSATGINRQGNTLDWYTRVLDAIAADED
ARKISYMLTWANFGWPNNMYVPYRDIHNELGGDHELLPDFEAFHADDYTAFRDEIK
GKIYNTGKEYTVSPHEPFMYVISPITGSTVTSETVTIQAKVANDEHARVTFRVDGSSL
EEEMVFNDDTLYYTGSFTPDAAVNGGAVDVIVAYYSSGEKVQEETIRLFVKIPEMSL
LTLTFDDDINGIKSNGTWPEDGVTSEIDHAIVDGDGKLMFSVQGMSPTETWQELKLE
LTELSDVNIDAVKKMKFDALIPAGSEEGSVOGIVOLPPDWETKYGMNETTKSIKDLE
TVTVNGSDYKRLEVTVSIDNQGGATGIALSLVGSQLDLLEPVYIDNIELLNSFEAPPA
DSFLVDDFEGYFG

The amino acid sequence of the mature form of Bsp Man4v3 is set forth as SEQ ID NO: 38.

(SEQ ID NO: 38)
SQEGRQLNMADEDASKYTKELFAFLQDVSGSQVLFGQQHATDEGLTLTNP
APRTGSTQSEVFNAVGDYPAVFGWDTNSLDGREKPGIAGNVEQSIKNTAQ
SMKVAHDLGGIITLSMHPDNFVTGGPYGDTTGNVVKEILPGGSKHAEFNA
WLDNIAALAHELKDENGEPIPMIFRPFHEQTGSWFWWGASTTSPEQYKAI
FRYTVEYLRDVKGVNNILYGFSPGAGPAGDVNRYLETYPGDDYVDIFGID
NYDNKDNAGSEAWLSGMVKDLAMISRLAEQKEKVAAFTEYGYSATGINRQ
GNTLDWYTRVLDAIAADEDARKISYMLTWANFGWPNNMYVPYRDIHNELG
GDHELLPDFEAFHADDYTAFRDEIKGKIYNTGKEYTVSPHEPFMYVISPI
TGSTVTSETVTIQAKVANDEHARVTFRVDGSSLEEEMVFNDDTLYYTGSF
TPDAAVNGGAVDVIVAYYSSGEKVQEETIRLFVKIPEMSLLTLTFDDDIN
GIKSNGTWPEDGVTSEIDHAIVDGDGKLMFSVQGMSPTETWQELKLELTE
LSDVNIDAVKKMKFDALIPAGSEEGSVQGIVQLPPDWETKYGMNETTKSI
KDLETVTVNGSDYKRLEVTVSIDNQGGATGIALSLVGSQLDLLEPVYIDN
IELLNSFEAPPADSFLVDDFEGYFG

The nucleotide sequence of the Bacillus sp. mannanase Bsp Man4v4 gene is set forth as SEQ ID NO: 39.

(SEQ ID NO: 39)
TCACAAGAAGGGCGTCAACTTAACATGGCAGATGAGGATGCTTCAAAGTA
TACGAAGGAGTTATTTGCTTTTCTTCAAGATGTAAGTGGTTCACAAGTGT
TATTTGGACAACAGCATGCAACAGATGAAGGATTAACTTTAACAAATCCA
GCTCCAAGAACAGGTTCCACTCAATCTGAAGTTTTCAATGCAGTTGGGGA
TTATCCAGCTGTGTTTGGATGGGACACGAATAGCCTAGATGGTCGTGAAA
AGCCTGGCATTGCAGGTAATGTAGAACAAAGTATAAAAAATACGGCTCAG
TCCATGAAAGTGGCTCATGATTTAGGAGGGATTATTACACTAAGCATGCA
CCCAGATAATTTTGTAACAGGGGGTCCTTATGGTGATACAACAGGGAATG
TTGTAAAAGAAATTCTTCCAGGTGGATCAAAACATGCAGAGTTTAACGCG
TGGTTGGACAATATTGCTGCGCTTGCTCACGAGCTGAAAGATGAGAATGG
TGAACCTATTCCGATGATTTTTCGGCCATTCCATGAACAAACAGGATCTT
GGTTTTGGTGGGGAGCAAGCACAACTTCACCCGAACAATATAAAGCGATT
TTTCGTTATACAGTAGAATATTTGCGAGATGTTAAAGGCGTAAATAATAT
TTTATATGGCTTTTCACCTGGGGCGGGACCTGCTGGAGATGTAAATCGCT
ATTTAGAAACATATCCAGGGGATGATTACGTTGATATTTTCGGTATTGAC
AATTATGACAATAAAGACAATGCAGGGTCAGAAGCTTGGTTAAGTGGTAT
GGTCAAAGACTTGGCGATGATTAGCCGATTAGCTGAACAAAAAGAAAAAG
TAGCGGCTTTTACTGAGTATGGGTACAGTGCAACCGGAATTAATCGTCAA
GGGAATACATTAGACTGGTACACACGTGTATTAGATGCGATTGCTGCTGA
TGAAGACGCACGTAAAATATCATACATGTTGACATGGGCGAACTTTGGTT
GGCCGAATAATATGTATGTTCCTTATCGTGATATCCACAATGAATTAGGT
GGAGACCATGAGTTATTACCGGACTTTGAAGCTTTCCATGCGGATGACTA
CACAGCATTTCGAGATGAGATAAAAGGAAAGATATATAATACTGGAAAGG
AATATACCGTTTCTCCTCATGAGCCGTTTATGTATGTTATATCTCCGATT
ACAGGTTCTACAGTGACAAGCGAAACGGTAACAATCCAAGCAAAAGTAGC
GAATGACGAACACGCAAGAGTCACTTTCAGGGTCGATGGTTCTAGTTTGG
AAGAAGAAATGGTTTTCAATGATGACACTTTATATTATACAGGTTCTTTT
ACACCAGATGCAGCAGTGAATGGCGGAGCTGTTGATGTGATTGTAGCTTA
TTATTCTAGTGGAGAAAAAGTCCAAGAAGAAACAATTCGTTTATTTGTAA
AAATTCCTGAAATGTCTTTGTTAACATTAACGTTTGATGATGATATAAAC
GGAATCAAAAGCAATGGAACATGGCCTGAAGATGGTGTAACATCTGAAAT
TGACCACGCTATTGTAGATGGAGACGGCAAGTTGATGTTCTCTGTTCAAG
GAATGTCACCTACTGAAACATGGCAAGAGCTCAAGTTAGAATTAACAGAA
CTATCAGATGTGAACATTGATGCGGTTAAGAAAATGAAGTTTGACGCGCT
TATCCCAGCAGGTAGTGAAGAAGGTTCAGTCCAAGGAATCGTACAACTTC
CACCGGATTGGGAGACGAAATATGGGATGAATGAAACAACGAAGTCAATA
AAAGACTTAGAGACTGTTACTGTTAATGGAAGCGATTATAAACGGTTGGA
AGTGACTGTTTCTATCGACAATCAAGGAGGAGCTACAGGAATCGCTTTAT
CATTAGTAGGATCCCAACTCGATTTGTTAGAACCTGTCTACATCGATAAT
ATTGAACTTCTAAATTCCTTTGAAGCACCACCAGCAGATTCTTTTCTTGT
TGATGATTTTGAAGGTTATTTTGGGGATGACACGTTGTTACATCGCAATT
ATTCTAGCAATGGAGATCCAATTACACTATCGTTAACAAGTGAGTTTAAA
AATAATGGAGAATTTGGATTGAAGTATGATTATTCGATTGGCTCGATGGG
TTATGCAGGGAGGCAAACATCACTAGGACCTGTCGATTGGAGCGGAGCTA
ATGCTTTTGAATTTTGGATGAAACATGGACAACTTGAAGGGAATCATTTA
ACTGTACAAATTCGAATAGGTGATGTTAGCTTTGAAAAAAATCTTGAATT
AATGGATGCTCATGAAGGTGTAGTGACAATCCCGTTTTCTGAATTTGCTC
CAGCTGCTTGGGAAAATAAGCCTGGCGTTATCATTGACGAACAAAAATTG
AAAAGAGTGAGTCAATTTGCTCTTTACACAGGCGGGGCTAGACAATCTGG
AACAATCTACTTTGATGATTTACGAGCGGTATATGATGAAAGTTTACCAT
CAGTTCCAGTTCCGAAAGAGGAGGAAGAGGAAAAAGAGGTCGCTCCTATT
TAA

The amino acid sequence of the Bacillus sp. mannanase Bsp Man4v4 protein is set forth as SEQ ID NO.40. The signal peptide is shown in italics and lowercase. There is a restriction enzyme site introduced between signal peptide and first codon of Bacillus sp. mannanase Bsp Man4v4, which is shown in lowercase and underline.

(SEQ ID NO: 40)
MrskklwisllfaltliftmafsnmsaqatsSQEGRQLNMADEDASKYTKELFAFLQDVSGSQVLFGQ
QHATDEGLTLTNPAPRTGSTQSEVFNAVGDYPAVFGWDTNSLDGREKPGIAGNVEQ
SIKNTAQSMKVAHDLGGIITLSMHPDNFVTGGPYGDTTGNVVKEILPGGSKHAEFNA
WLDNIAALAHELKDENGEPIPMIFRPFHEQTGSWFWWGASTTSPEQYKAIFRYTVEY
LRDVKGVNNILYGFSPGAGPAGDVNRYLETYPGDDYVDIFGIDNYDNKDNAGSEAW
LSGMVKDLAMISRLAEQKEKVAAFTEYGYSATGINRQGNTLDWYTRVLDAIAADED
ARKISYMLTWANFGWPNNMYVPYRDIHNELGGDHELLPDFEAFHADDYTAFRDEIK
GKIYNTGKEYTVSPHEPFMYVISPITGSTVTSETVTIQAKVANDEHARVTFRVDGSSL
EEEMVFNDDTLYYTGSFTPDAAVNGGAVDVIVAYYSSGEKVQEETIRLFVKIPEMSL
LTLTFDDDINGIKSNGTWPEDGVTSEIDHAIVDGDGKLMFSVQGMSPTETWQELKLE
LTELSDVNIDAVKKMKFDALIPAGSEEGSVQGIVQLPPDWETKYGMNETTKSIKDLE
TVTVNGSDYKRLEVTVSIDNQGGATGIALSLVGSQLDLLEPVYIDNIELLNSFEAPPA
DSFLVDDFEGYFGDDTLLHRNYSSNGDPITLSLTSEFKNNGEFGLKYDYSIGSMGYA
GRQTSLGPVDWSGANAFEFWMKHGQLEGNHLTVQIRIGDVSFEKNLELMDAHEGV
VTIPFSEFAPAAWENKPGVIIDEQKLKRVSQFALYTGGARQSGTIYFDDLRAVYDESL
PSVPVPKEEEEEKEVAPI

The amino acid sequence of the mature form of Bsp Man4v4 is set forth as SEQ ID NO: 41.

(SEQ ID NO: 41)
SQEGRQLNMADEDASKYTKELFAFLQDVSGSQVLFGQQHATDEGLTLTNP
APRTGSTQSEVFNAVGDYPAVFGWDTNSLDGREKPGIAGNVEQSIKNTAQ
SMKVAHDLGGIITLSMHPDNFVTGGPYGDTTGNVVKEILPGGSKHAEFNA
WLDNIAALAHELKDENGEPIPMIFRPFHEQTGSWFWWGASTTSPEQYKAI
FRYTVEYLRDVKGVNNILYGFSPGAGPAGDVNRYLETYPGDDYVDIFGID
NYDNKDNAGSEAWLSGMVKDLAMISRLAEQKEKVAAFTEYGYSATGINRQ
GNTLDWYTRVLDAIAADEDARKISYMLTWANFGWPNNMYVPYRDIHNELG
GDHELLPDFEAFHADDYTAFRDEIKGKIYNTGKEYTVSPHEPFMYVISPI
TGSTVTSETVTIQAKVANDEHARVTFRVDGSSLEEEMVFNDDTLYYTGSF
TPDAAVNGGAVDVIVAYYSSGEKVQEETIRLFVKIPEMSLLTLTFDDDIN
GIKSNGTWPEDGVTSEIDHAIVDGDGKLMFSVQGMSPTETWQELKLELTE
LSDVNIDAVKKMKFDALIPAGSEEGSVQGIVQLPPDWETKYGMNETTKSI
KDLETVTVNGSDYKRLEVTVSIDNQGGATGIALSLVGSQLDLLEPVYIDN
IELLNSFEAPPADSFLVDDFEGYFGDDTLLHRNYSSNGDPITLSLTSEFK
NNGEFGLKYDYSIGSMGYAGRQTSLGPVDWSGANAFEFWMKHGQLEGNHL
TVQIRIGDVSFEKNLELMDAHEGVVTIPFSEFAPAAWENKPGVIIDEQKL
KRVSQFALYTGGARQSGTIYFDDLRAVYDESLPSVPVPKEEEEEKEVAPI

### Example 10

### Expression of Bsp Man4 Deletion Variants

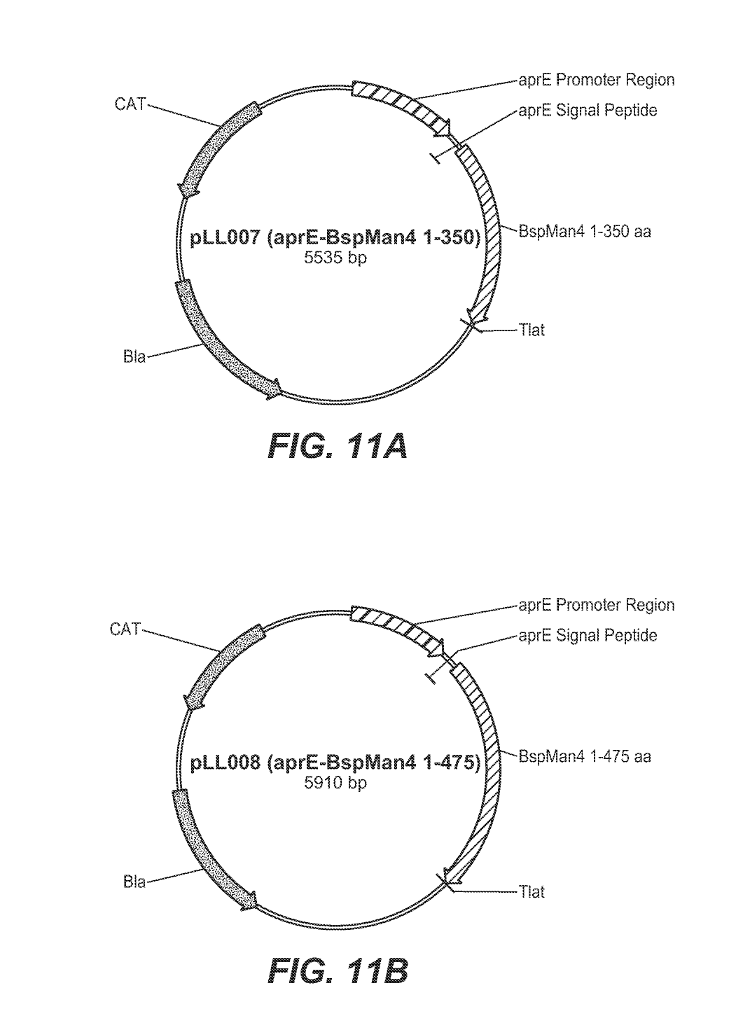

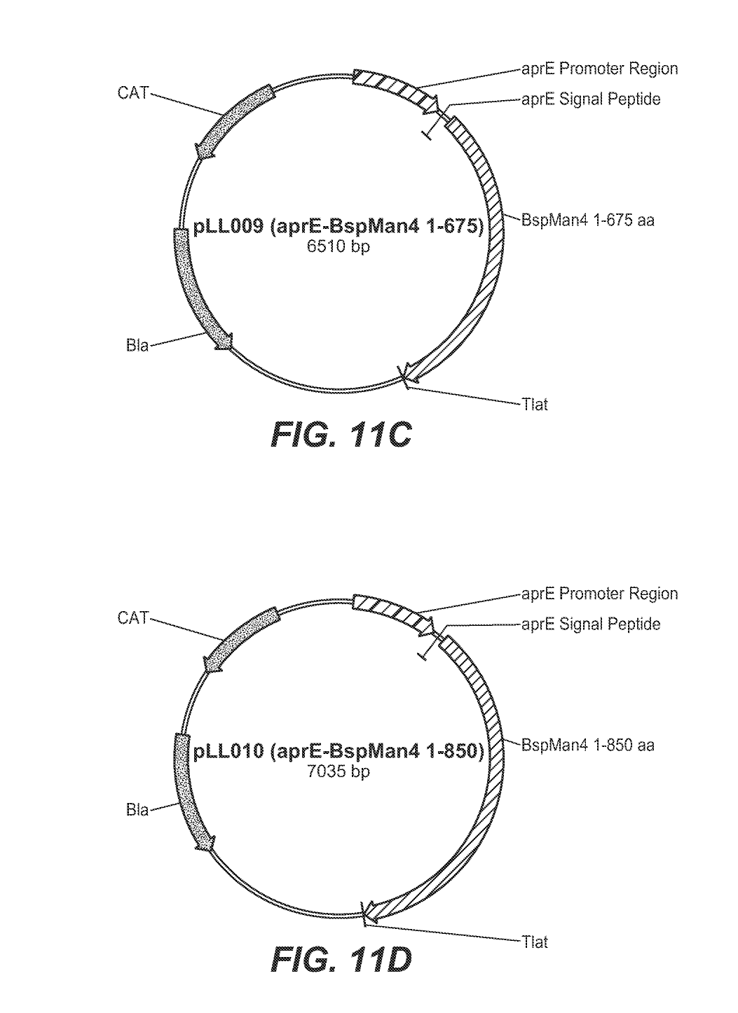

Bsp Man4v1, Bsp Man4v2, Bsp Man4v3 and Bsp Man4v4 PCR products were cloned into p2JM expression vector and the resulting plasmid were labeled as pLL007 (aprE-Bsp Man4 1-350), pLL008 (aprE-Bsp Man4 1-475), pLL009 (aprE-Bsp Man4 1-675) and pLL010 (aprE-Bsp Man4 1-850). Plasmid maps are provided in FIG. 11. The sequence of the deletion version of genes was confirmed by DNA sequencing.

The plasmid pLL007 (aprE-Bsp Man4 1-350), pLL008 (aprE-Bsp Man4 1-475), pLL009 (aprE-Bsp Man4 1-675) and pLL010 (aprE-Bsp Man4 1-850) are amplified using rolling circle kit (GE Healthcare Life Sciences, NJ) before transformations. Bacillus subtilis (degUHy32, AnprB, Avpr, Aepr, AscoC, AwprA, Ampr, AispA, Abpr) were transformed with the amplified plasmid. The transformed cells were then plated on Luria Agar plates supplemented with 10 ppm kanamycin. Single colony were picked and cultured in shake flasks.

The nucleotide sequence of Bsp Man4 v1 gene from expression plasmid pLL007 (aprE-Bsp Man4 1-350) is set forth as SEQ ID NO:42. The signal sequence is shown in bold.

(SEQ ID NO: 42)
GTGAGAAGCAAAAAATTGTGGATCAGCTTGTTGTTTGCGTTAACGTTAAT
CTTTACGATGGCGTTCAGCAACATGAGCGCGCAGGCAGCTGGTAAAACTA
GTTCACAAGAAGGGCGTCAACTTAACATGGCAGATGAGGATGCTTCAAAG
TATACGAAGGAGTTATTTGCTTTTCTTCAAGATGTAAGTGGTTCACAAGT
GTTATTTGGACAACAGCATGCAACAGATGAAGGATTAACTTTAACAAATC
CAGCTCCAAGAACAGGTTCCACTCAATCTGAAGTTTTCAATGCAGTTGGG
GATTATCCAGCTGTGTTTGGATGGGACACGAATAGCCTAGATGGTCGTGA
AAAGCCTGGCATTGCAGGTAATGTAGAACAAAGTATAAAAAATACGGCTC
AGTCCATGAAAGTGGCTCATGATTTAGGAGGGATTATTACACTAAGCATG
CACCCAGATAATTTTGTAACAGGGGGTCCTTATGGTGATACAACAGGGAA
TGTTGTAAAAGAAATTCTTCCAGGTGGATCAAAACATGCAGAGTTTAACG
CGTGGTTGGACAATATTGCTGCGCTTGCTCACGAGCTGAAAGATGAGAAT
GGTGAACCTATTCCGATGATTTTTCGGCCATTCCATGAACAAACAGGATC
TTGGTTTTGGTGGGGAGCAAGCACAACTTCACCCGAACAATATAAAGCGA
TTTTTCGTTATACAGTAGAATATTTGCGAGATGTTAAAGGCGTAAATAAT
ATTTTATATGGCTTTTCACCTGGGGCGGGACCTGCTGGAGATGTAAATCG
CTATTTAGAAACATATCCAGGGGATGATTACGTTGATATTTTCGGTATTG
ACAATTATGACAATAAAGACAATGCAGGGTCAGAAGCTTGGTTAAGTGGT
ATGGTCAAAGACTTGGCGATGATTAGCCGATTAGCTGAACAAAAAGAAAA
AGTAGCGGCTTTTACTGAGTATGGGTACAGTGCAACCGGAATTAATCGTC
AAGGGAATACATTAGACTGGTACACACGTGTATTAGATGCGATTGCTGCT
GATGAAGACGCACGTAAAATATCATACATGTTGACATGGGCGAACTTTGG
TTGGCCGAATAATATGTATGTTCCTTATCGTGATATCCACAATGAATTAG
GTTAA

The amino acid of Bsp Man4 v1 protein from expression plasmid pLL007 (aprE-Bsp Man4 1-350) is set forth as SEQ ID NO:43. The signal sequence is shown in bold.

(SEQ ID NO: 43)
vrskklwisllfaltliftmafsnmsaqaAGKTSSQEGRQLNMADEDASKYTKELFAFLQDVSGSQV
LFGQQHATDEGLTLTNPAPRTGSTQSEVFNAVGDYPAVFGWDTNSLDGREKPGIAG
NVEQSIKNTAQSMKVAHDLGGIITLSMHPDNFVTGGPYGDTTGNVVKEILPGGSKHA
EFNAWLDNIAALAHELKDENGEPIPMIFRPFHEQTGSWFWWGASTTSPEQYKAIFRY
TVEYLRDVKGVNNILYGFSPGAGPAGDVNRYLETYPGDDYVDIFGIDNYDNKDNAG
SEAWLSGMVKDLAMISRLAEQKEKVAAFTEYGYSATGINRQGNTLDWYTRVLDAIA
ADEDARKISYMLTWANFGWPNNMYVPYRDIHNELG

The nucleotide sequence of Bsp Man4 v2 gene from expression plasmid pLL008 (aprE-Bsp Man4 1-475) is set forth as SEQ ID NO:44. The signal sequence is shown in bold.

(SEQ ID NO: 44)
GTGAGAAGCAAAAAATTGTGGATCAGCTTGTTGTTTGCGTTAACGTTAAT
CTTTACGATGGCGTTCAGCAACATGAGCGCGCAGGCAGCTGGTAAAACTA
GTTCACAAGAAGGGCGTCAACTTAACATGGCAGATGAGGATGCTTCAAAG
TATACGAAGGAGTTATTTGCTTTTCTTCAAGATGTAAGTGGTTCACAAGT
GTTATTTGGACAACAGCATGCAACAGATGAAGGATTAACTTTAACAAATC
CAGCTCCAAGAACAGGTTCCACTCAATCTGAAGTTTTCAATGCAGTTGGG
GATTATCCAGCTGTGTTTGGATGGGACACGAATAGCCTAGATGGTCGTGA
AAAGCCTGGCATTGCAGGTAATGTAGAACAAAGTATAAAAAATACGGCTC
AGTCCATGAAAGTGGCTCATGATTTAGGAGGGATTATTACACTAAGCATG
CACCCAGATAATTTTGTAACAGGGGGTCCTTATGGTGATACAACAGGGAA
TGTTGTAAAAGAAATTCTTCCAGGTGGATCAAAACATGCAGAGTTTAACG
CGTGGTTGGACAATATTGCTGCGCTTGCTCACGAGCTGAAAGATGAGAAT
GGTGAACCTATTCCGATGATTTTTCGGCCATTCCATGAACAAACAGGATC
TTGGTTTTGGTGGGGAGCAAGCACAACTTCACCCGAACAATATAAAGCGA
TTTTTCGTTATACAGTAGAATATTTGCGAGATGTTAAAGGCGTAAATAAT
ATTTTATATGGCTTTTCACCTGGGGCGGGACCTGCTGGAGATGTAAATCG
CTATTTAGAAACATATCCAGGGGATGATTACGTTGATATTTTCGGTATTG
ACAATTATGACAATAAAGACAATGCAGGGTCAGAAGCTTGGTTAAGTGGT
ATGGTCAAAGACTTGGCGATGATTAGCCGATTAGCTGAACAAAAAGAAAA
AGTAGCGGCTTTTACTGAGTATGGGTACAGTGCAACCGGAATTAATCGTC
AAGGGAATACATTAGACTGGTACACACGTGTATTAGATGCGATTGCTGCT
GATGAAGACGCACGTAAAATATCATACATGTTGACATGGGCGAACTTTGG
TTGGCCGAATAATATGTATGTTCCTTATCGTGATATCCACAATGAATTAG
GTGGAGACCATGAGTTATTACCGGACTTTGAAGCTTTCCATGCGGATGAC
TACACAGCATTTCGAGATGAGATAAAAGGAAAGATATATAATACTGGAAA
GGAATATACCGTTTCTCCTCATGAGCCGTTTATGTATGTTATATCTCCGA
TTACAGGTTCTACAGTGACAAGCGAAACGGTAACAATCCAAGCAAAAGTA
GCGAATGACGAACACGCAAGAGTCACTTTCAGGGTCGATGGTTCTAGTTT
GGAAGAAGAAATGGTTTTCAATGATGACACTTTATATTATACAGGTTCTT
TTACACCAGATGCAGCAGTGAATGGCGGAGCTGTTGATGTGATTGTAGCT
TATTATTCTAGTGGAGAAAAAGTCCAATAA

The amino acid of Bsp Man4 v2 protein from expression plasmid pLL008 (aprE-Bsp Man4 1-475) is set forth as SEQ ID NO:45. The signal sequence is shown in bold.

(SEQ ID NO: 45)
vrskklwisllfaltliftmafsnmsaqaAGKTSSQEGRQLNMADEDASKYTKELFAFLQDVSGSQV
LFGQQHATDEGLTLTNPAPRTGSTQSEVFNAVGDYPAVFGWDTNSLDGREKPGIAG
NVEQSIKNTAQSMKVAHDLGGIITLSMHPDNFVTGGPYGDTTGNVVKEILPGGSKHA
EFNAWLDNIAALAHELKDENGEPIPMIFRPFHEQTGSWFWWGASTTSPEQYKAIFRY
TVEYLRDVKGVNNILYGFSPGAGPAGDVNRYLETYPGDDYVDIFGIDNYDNKDNAG
SEAWLSGMVKDLAMISRLAEQKEKVAAFTEYGYSATGINRQGNTLDWYTRVLDAIA
ADEDARKISYMLTWANFGWPNNMYVPYRDIHNELGGDHELLPDFEAFHADDYTAF
RDEIKGKIYNTGKEYTVSPHEPFMYVISPITGSTVTSETVTIQAKVANDEHARVTFRV
DGSSLEEEMVFNDDTLYYTGSFTPDAAVNGGAVDVIVAYYSSGEKVQ

The nucleotide sequence of Bsp Man4 v3 gene from expression plasmid pLL009 (aprE-Bsp Man4 1-675) is set forth as SEQ ID NO:46. The signal sequence is shown in bold.

(SEQ ID NO: 46)
GTGAGAAGCAAAAAATTGTGGATCAGCTTGTTGTTTGCGTTAACGTTAAT
CTTTACGATGGCGTTCAGCAACATGAGCGCGCAGGCAGCTGGTAAAACTA
GTTCACAAGAAGGGCGTCAACTTAACATGGCAGATGAGGATGCTTCAAAG
TATACGAAGGAGTTATTTGCTTTTCTTCAAGATGTAAGTGGTTCACAAGT
GTTATTTGGACAACAGCATGCAACAGATGAAGGATTAACTTTAACAAATC
CAGCTCCAAGAACAGGTTCCACTCAATCTGAAGTTTTCAATGCAGTTGGG
GATTATCCAGCTGTGTTTGGATGGGACACGAATAGCCTAGATGGTCGTGA
AAAGCCTGGCATTGCAGGTAATGTAGAACAAAGTATAAAAAATACGGCTC
AGTCCATGAAAGTGGCTCATGATTTAGGAGGGATTATTACACTAAGCATG
CACCCAGATAATTTTGTAACAGGGGGTCCTTATGGTGATACAACAGGGAA
TGTTGTAAAAGAAATTCTTCCAGGTGGATCAAAACATGCAGAGTTTAACG
CGTGGTTGGACAATATTGCTGCGCTTGCTCACGAGCTGAAAGATGAGAAT
GGTGAACCTATTCCGATGATTTTTCGGCCATTCCATGAACAAACAGGATC
TTGGTTTTGGTGGGGAGCAAGCACAACTTCACCCGAACAATATAAAGCGA
TTTTTCGTTATACAGTAGAATATTTGCGAGATGTTAAAGGCGTAAATAAT
ATTTTATATGGCTTTTCACCTGGGGCGGGACCTGCTGGAGATGTAAATCG
CTATTTAGAAACATATCCAGGGGATGATTACGTTGATATTTTCGGTATTG
ACAATTATGACAATAAAGACAATGCAGGGTCAGAAGCTTGGTTAAGTGGT
ATGGTCAAAGACTTGGCGATGATTAGCCGATTAGCTGAACAAAAAGAAAA
AGTAGCGGCTTTTACTGAGTATGGGTACAGTGCAACCGGAATTAATCGTC
AAGGGAATACATTAGACTGGTACACACGTGTATTAGATGCGATTGCTGCT
GATGAAGACGCACGTAAAATATCATACATGTTGACATGGGCGAACTTTGG
TTGGCCGAATAATATGTATGTTCCTTATCGTGATATCCACAATGAATTAG
GTGGAGACCATGAGTTATTACCGGACTTTGAAGCTTTCCATGCGGATGAC
TACACAGCATTTCGAGATGAGATAAAAGGAAAGATATATAATACTGGAAA
GGAATATACCGTTTCTCCTCATGAGCCGTTTATGTATGTTATATCTCCGA
TTACAGGTTCTACAGTGACAAGCGAAACGGTAACAATCCAAGCAAAAGTA
GCGAATGACGAACACGCAAGAGTCACTTTCAGGGTCGATGGTTCTAGTTT
GGAAGAAGAAATGGTTTTCAATGATGACACTTTATATTATACAGGTTCTT
TTACACCAGATGCAGCAGTGAATGGCGGAGCTGTTGATGTGATTGTAGCT
TATTATTCTAGTGGAGAAAAAGTCCAAGAAGAAACAATTCGTTTATTTGT
AAAAATTCCTGAAATGTCTTTGTTAACATTAACGTTTGATGATGATATAA
ACGGAATCAAAAGCAATGGAACATGGCCTGAAGATGGTGTAACATCTGAA
ATTGACCACGCTATTGTAGATGGAGACGGCAAGTTGATGTTCTCTGTTCA
AGGAATGTCACCTACTGAAACATGGCAAGAGCTCAAGTTAGAATTAACAG
AACTATCAGATGTGAACATTGATGCGGTTAAGAAAATGAAGTTTGACGCG
CTTATCCCAGCAGGTAGTGAAGAAGGTTCAGTCCAAGGAATCGTACAACT
TCCACCGGATTGGGAGACGAAATATGGGATGAATGAAACAACGAAGTCAA
TAAAAGACTTAGAGACTGTTACTGTTAATGGAAGCGATTATAAACGGTTG
GAAGTGACTGTTTCTATCGACAATCAAGGAGGAGCTACAGGAATCGCTTT
ATCATTAGTAGGATCCCAACTCGATTTGTTAGAACCTGTCTACATCGATA
ATATTGAACTTCTAAATTCCTTTGAAGCACCACCAGCAGATTCTTTTCTT
GTTGATGATTTTGAAGGTTATTTTGGGTAA

The amino acid of Bsp Man4 v3 protein from expression plasmid pLL009 (aprE-Bsp Man4 1-675) is set forth as SEQ ID NO:47. The signal sequence is shown in bold.

(SEQ ID NO: 47)
vrskklwisllfaltliftmafsnmsaqaAGKTSSQEGRQLNMADEDASKYTKELFAFLQDVSGSQV
LFGQQHATDEGLTLTNPAPRTGSTQSEVFNAVGDYPAVFGWDTNSLDGREKPGIAG
NVEQSIKNTAQSMKVAHDLGGIITLSMHPDNFVTGGPYGDTTGNVVKEILPGGSKHA
EFNAWLDNIAALAHELKDENGEPIPMIFRPFHEQTGSWFWWGASTTSPEQYKAIFRY
TVEYLRDVKGVNNILYGFSPGAGPAGDVNRYLETYPGDDYVDIFGIDNYDNKDNAG
SEAWLSGMVKDLAMISRLAEQKEKVAAFTEYGYSATGINRQGNTLDWYTRVLDAIA
ADEDARKISYMLTWANFGWPNNMYVPYRDIHNELGGDHELLPDFEAFHADDYTAF
RDEIKGKIYNTGKEYTVSPHEPFMYVISPITGSTVTSETVTIQAKVANDEHARVTFRV
DGSSLEEEMVFNDDTLYYTGSFTPDAAVNGGAVDVIVAYYSSGEKVQEETIRLFVKI
PEMSLLTLTFDDDINGIKSNGTWPEDGVTSEIDHAIVDGDGKLMFSVQGMSPTETWQ
ELKLELTELSDVNIDAVKKMKFDALIPAGSEEGSVQGIVQLPPDWETKYGMNETTKS
IKDLETVTVNGSDYKRLEVTVSIDNQGGATGIALSLVGSQLDLLEPVYIDNIELLNSFE
APPADSFLVDDFEGYFG

The nucleotide sequence of Bsp Man4 v4 gene from expression plasmid pLL010 (aprE-Bsp Man4 1-850) is set forth as SEQ ID NO:48. The signal sequence is shown in bold.

(SEQ ID NO: 48)
GTGAGAAGCAAAAAATTGTGGATCAGCTTGTTGTTTGCGTTAACGTTAAT
CTTTACGATGGCGTTCAGCAACATGAGCGCGCAGGCAGCTGGTAAAACTA
GTTCACAAGAAGGGCGTCAACTTAACATGGCAGATGAGGATGCTTCAAAG
TATACGAAGGAGTTATTTGCTTTTCTTCAAGATGTAAGTGGTTCACAAGT
GTTATTTGGACAACAGCATGCAACAGATGAAGGATTAACTTTAACAAATC
CAGCTCCAAGAACAGGTTCCACTCAATCTGAAGTTTTCAATGCAGTTGGG
GATTATCCAGCTGTGTTTGGATGGGACACGAATAGCCTAGATGGTCGTGA
AAAGCCTGGCATTGCAGGTAATGTAGAACAAAGTATAAAAAATACGGCTC
AGTCCATGAAAGTGGCTCATGATTTAGGAGGGATTATTACACTAAGCATG
CACCCAGATAATTTTGTAACAGGGGGTCCTTATGGTGATACAACAGGGAA
TGTTGTAAAAGAAATTCTTCCAGGTGGATCAAAACATGCAGAGTTTAACG
CGTGGTTGGACAATATTGCTGCGCTTGCTCACGAGCTGAAAGATGAGAAT
GGTGAACCTATTCCGATGATTTTTCGGCCATTCCATGAACAAACAGGATC
TTGGTTTTGGTGGGGAGCAAGCACAACTTCACCCGAACAATATAAAGCGA
TTTTTCGTTATACAGTAGAATATTTGCGAGATGTTAAAGGCGTAAATAAT
ATTTTATATGGCTTTTCACCTGGGGCGGGACCTGCTGGAGATGTAAATCG
CTATTTAGAAACATATCCAGGGGATGATTACGTTGATATTTTCGGTATTG
ACAATTATGACAATAAAGACAATGCAGGGTCAGAAGCTTGGTTAAGTGGT
ATGGTCAAAGACTTGGCGATGATTAGCCGATTAGCTGAACAAAAAGAAAA
AGTAGCGGCTTTTACTGAGTATGGGTACAGTGCAACCGGAATTAATCGTC
AAGGGAATACATTAGACTGGTACACACGTGTATTAGATGCGATTGCTGCT
GATGAAGACGCACGTAAAATATCATACATGTTGACATGGGCGAACTTTGG
TTGGCCGAATAATATGTATGTTCCTTATCGTGATATCCACAATGAATTAG
GTGGAGACCATGAGTTATTACCGGACTTTGAAGCTTTCCATGCGGATGAC
TACACAGCATTTCGAGATGAGATAAAAGGAAAGATATATAATACTGGAAA
GGAATATACCGTTTCTCCTCATGAGCCGTTTATGTATGTTATATCTCCGA
TTACAGGTTCTACAGTGACAAGCGAAACGGTAACAATCCAAGCAAAAGTA
GCGAATGACGAACACGCAAGAGTCACTTTCAGGGTCGATGGTTCTAGTTT
GGAAGAAGAAATGGTTTTCAATGATGACACTTTATATTATACAGGTTCTT
TTACACCAGATGCAGCAGTGAATGGCGGAGCTGTTGATGTGATTGTAGCT
TATTATTCTAGTGGAGAAAAAGTCCAAGAAGAAACAATTCGTTTATTTGT
AAAAATTCCTGAAATGTCTTTGTTAACATTAACGTTTGATGATGATATAA
ACGGAATCAAAAGCAATGGAACATGGCCTGAAGATGGTGTAACATCTGAA
ATTGACCACGCTATTGTAGATGGAGACGGCAAGTTGATGTTCTCTGTTCA
AGGAATGTCACCTACTGAAACATGGCAAGAGCTCAAGTTAGAATTAACAG
AACTATCAGATGTGAACATTGATGCGGTTAAGAAAATGAAGTTTGACGCG
CTTATCCCAGCAGGTAGTGAAGAAGGTTCAGTCCAAGGAATCGTACAACT
TCCACCGGATTGGGAGACGAAATATGGGATGAATGAAACAACGAAGTCAA
TAAAAGACTTAGAGACTGTTACTGTTAATGGAAGCGATTATAAACGGTTG
GAAGTGACTGTTTCTATCGACAATCAAGGAGGAGCTACAGGAATCGCTTT
ATCATTAGTAGGATCCCAACTCGATTTGTTAGAACCTGTCTACATCGATA
ATATTGAACTTCTAAATTCCTTTGAAGCACCACCAGCAGATTCTTTTCTT
GTTGATGATTTTGAAGGTTATTTTGGGGATGACACGTTGTTACATCGCAA
TTATTCTAGCAATGGAGATCCAATTACACTATCGTTAACAAGTGAGTTTA
AAAATAATGGAGAATTTGGATTGAAGTATGATTATTCGATTGGCTCGATG
GGTTATGCAGGGAGGCAAACATCACTAGGACCTGTCGATTGGAGCGGAGC
TAATGCTTTTGAATTTTGGATGAAACATGGACAACTTGAAGGGAATCATT
TAACTGTACAAATTCGAATAGGTGATGTTAGCTTTGAAAAAAATCTTGAA
TTAATGGATGCTCATGAAGGTGTAGTGACAATCCCGTTTTCTGAATTTGC
TCCAGCTGCTTGGGAAAATAAGCCTGGCGTTATCATTGACGAACAAAAAT
TGAAAAGAGTGAGTCAATTTGCTCTTTACACAGGCGGGGCTAGACAATCT
GGAACAATCTACTTTGATGATTTACGAGCGGTATATGATGAAAGTTTACC
ATCAGTTCCAGTTCCGAAAGAGGAGGAAGAGGAAAAAGAGGTCGCTCCTA
TTTAA

The amino acid of Bsp Man4 v4 protein from expression plasmid pLL009 (aprE-Bsp Man4 1-850) is set forth as SEQ ID NO:49. The signal sequence is shown in bold.

(SEQ ID NO: 49)
vrskklwisllfaltliftmafsnmsaqaAGKTSSQEGRQLNMADEDASKYTKELFAFLQDVSGSQV
LFGQQHATDEGLTLTNPAPRTGSTQSEVFNAVGDYPAVFGWDTNSLDGREKPGIAG
NVEQSIKNTAQSMKVAHDLGGIITLSMHPDNFVTGGPYGDTTGNVVKEILPGGSKHA
EFNAWLDNIAALAHELKDENGEPIPMIFRPFHEQTGSWFWWGASTTSPEQYKAIFRY
TVEYLRDVKGVNNILYGFSPGAGPAGDVNRYLETYPGDDYVDIFGIDNYDNKDNAG
SEAWLSGMVKDLAMISRLAEQKEKVAAFTEYGYSATGINRQGNTLDWYTRVLDAIA
ADEDARKISYMLTWANFGWPNNMYVPYRDIHNELGGDHELLPDFEAFHADDYTAF
RDEIKGKIYNTGKEYTVSPHEPFMYVISPITGSTVTSETVTIQAKVANDEHARVTFRV
DGSSLEEEMVFNDDTLYYTGSFTPDAAVNGGAVDVIVAYYSSGEKVQEETIRLFVKI
PEMSLLTLTFDDDINGIKSNGTWPEDGVTSEIDHAIVDGDGKLMFSVQGMSPTETWQ
ELKLELTELSDVNIDAVKKMKFDALIPAGSEEGSVQGIVQLPPDWETKYGMNETTKS
IKDLETVTVNGSDYKRLEVTVSIDNQGGATGIALSLVGSQLDLLEPVYIDNIELLNSFE
APPADSFLVDDFEGYFGDDTLLHRNYSSNGDPITLSLTSEFKNNGEFGLKYDYSIGSM
GYAGRQTSLGPVDWSGANAFEFWMKHGQLEGNHLTVQIRIGDVSFEKNLELMDAH
EGVVTIPFSEFAPAAWENKPGVIIDEQKLKRVSQFALYTGGARQSGTIYFDDLRAVYD
ESLPSVPVPKEEEEEKEVAPI

### Example 11

### Purification of Bsp Man4v2

Bsp Man4v2 was purified via the hydrophobic interaction and anion-exchange chromatography. Ammonium sulphate was added to 700 mL crude broth from the shake flask to a final concentration of 1 M. The solution was then loaded onto a 150 mL phenyl-sepharose FF (high sub) XK 26/20 column pre-equilibrated with 20 mM sodium phosphate pH 6.0 (buffer A) with 1 M ammonium sulphate (buffer B). The target protein was eluted with 100%-0 buffer B in 3 CV, followed by 3 CV of MilliQ H₂O. The fractions containing target protein were pooled and desalted by ultrafiltration. The desalted sample was then loaded onto a 150 ml Q-Sepharose FF XK26/20 column pre-equilibrated with 20 mM Tris-HCl, pH 7.5 (buffer C). After sample loading, the column was washed with the same buffer for 2 column volumes, followed by a gradient of 0-80% buffer C with 1 M NaCl (buffer D) in 8 CVs. The fractions containing target protein were pooled and concentrated using 10K Amicon Ultra-15 devices. The sample was above 95% pure and stored in 40% glycerol at −80° C. until usage.

### Example 12

### Mannanase Activity of Bsp Man4 and Bsp Man4v2

The beta 1-4 mannanase activity of Bsp Man4 and Bsp Man4v2 was measured using 1% Galactomannan (Carob; Low Viscosity) (P-GALML; Lot 10501) purchased from Megazyme International Ireland (Bray, Ireland). The assay was performed in 50 mM sodium acetate pH 5.0, 0.005% Tween-80 buffer at 50° C. for 10 minutes or in 50 mM HEPES pH 8.2, 0.005% Tween-80 buffer at 30° C. for 30 minutes. The released reducing sugar was quantified in a PAHBAH (p-Hydroxy benzoic acid hydrazide) assay (Lever, Anal. Biochem. 47:248, 1972). A standard curve using mannose was generated and used to calculate enzyme activity units. In this assay, one mannanase unit is defined as the amount of enzyme required to generate 1 micromole of mannose reducing sugar equivalents per minute under the conditions of the assay. The specific activity of purified Bsp Man4 at pH 5.0 was determined to be 67 units/mg, whereas Bsp Man4v2 was determined to be 156 units/mg towards low viscosity carob galactomannan using the above method.

### Example 13

### pH Profiles of Bsp Man4v2

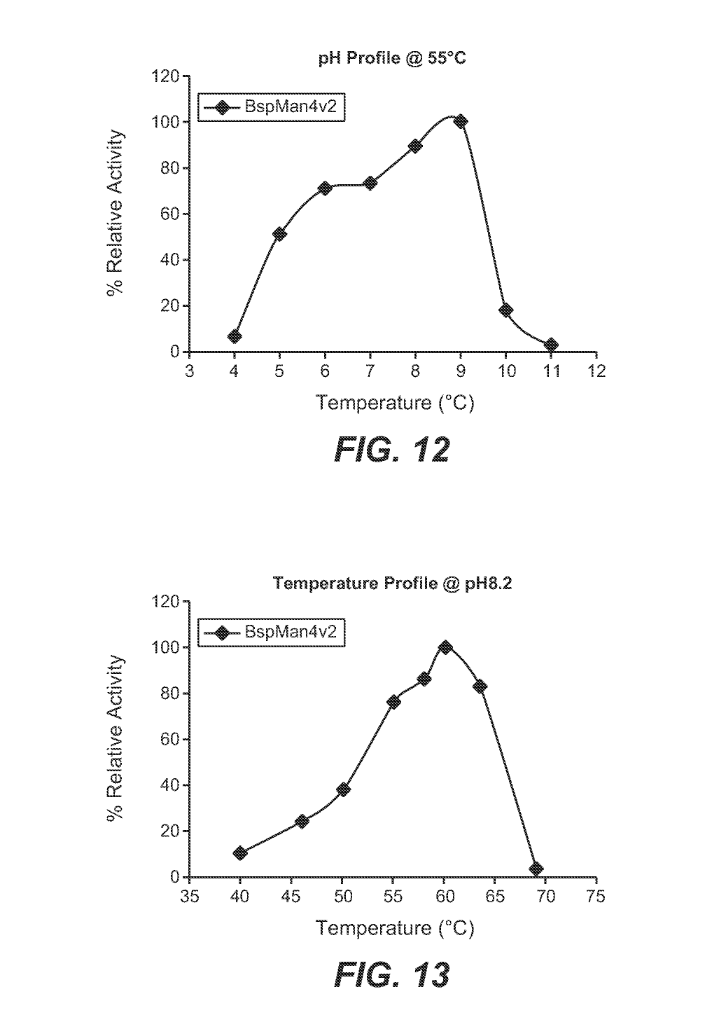

The pH profiles of Bsp Man4v2 were determined using the beta-mannazyme tablet assay from Megazyme (Tmnz 1/02; Azurine-crosslinked carob galactomannan) with minor modifications to the suggested protocol. The assay was performed in 50 mm Acetate/Bis-Tris/HEPES/CHES buffer adjusted to pH values between 4 and 11. The enzyme solution was diluted into the assay buffer and 500 μl of the enzyme solution was equilibrated at 55° C. before adding one substrate tablet. After 10 minutes, the reaction was stopped by adding 10 ml 2% Tris pH 12. The tubes were left at room temperature for 5 minutes, stirred and the liquid filtered through a Whatman No.1 paper filter. Release of blue dye from the substrate was quantified by measuring the optical density at 590 nm. Enzyme activity at each pH was reported as relative activity where the activity at the pH optimum was set to 100%. The pH profile of Bsp Man4v2 is shown in FIG. 12.

### Example 14

Temperature Profiles of Bsp Man4 and Bsp Man4v2 The temperature optimum of purified Bsp Man4v2 was determined by assaying for mannanase activity using the beta-mannazyme tablet assay from Megazyme (Tmnz 1/02; Azurine-crosslinked carob galactomannan) with minor modifications to the suggested protocol. The assay was performed at temperatures varying between 40° C. and 69° C. for 10 minutes in 50 mM HEPES buffer at pH 8.2. The activity was reported as relative activity where the activity at the temperature optimum was set to 100%. The temperature profile of Bsp Man4v2 is shown in FIG. 13.

### Example 15

### Thermostability of Bsp Man4 and Bsp Man4v2

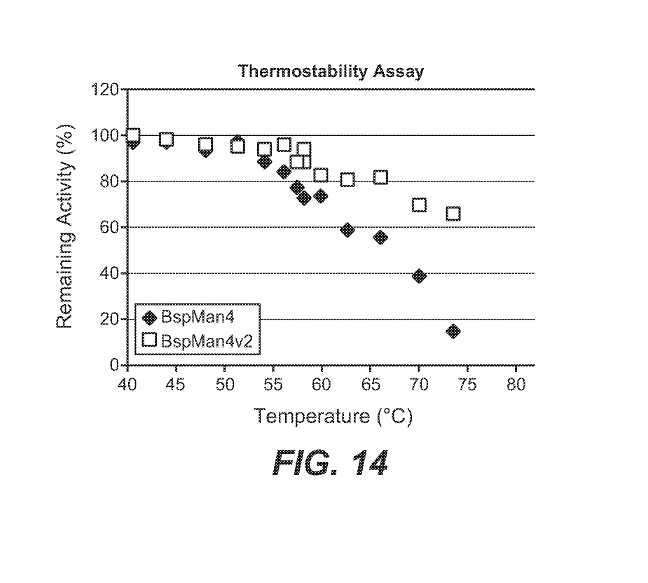

The thermostability of Bsp Man4 and Bsp Man4v2 was determined in 50 mM HEPES buffer pH 8.2. The enzymes were incubated at desired temperature for 2 hours in the Bio-Rad PCR machine. The remaining activity of the samples was measured using the Azo-Carob Galactomannan assay from Megazyme (ACGLM 03/07; Remazolbrilliant Blue R dyed carob galactomannan) with minor modifications to the suggested protocol. The activity of the sample kept on ice was defined as 100% activity. The thermostability results for Bsp Man4 and Bsp Man4v2 are shown in FIG. 14. At temperatures lower than 55° C., no activity loss was detected for either Bsp Man4 or Bsp Man4v2 during a 2-hour incubation. Bsp Man4v2 retains more activity than Bsp Man4 at the elevated temperatures.

### Example 16

### Liquid Laundry Detergent Compositions Comprising Bsp Man4

In this example, various formulations for liquid laundry detergent compositions are provided. In each of these formulations, Bsp Man4 is included at a concentration of from about 0.0001 to about 10 weight percent. In some alternative embodiments, other concentrations will find use, as determined by the formulator, based on their needs.

TABLE 16-1
Liquid Laundry Detergent Compositions

| | Formulations | | | | |
|---|---|---|---|---|---|
| Compound | I | II | III | IV | V |
| LAS | 24.0 | 32.0 | 6.0 | 3.0 | 6.0 |
| NaC₁₆-C₁₇HSAS | — | — | — | 5.0 | — |
| C₁₂-C₁₅AE_{1.8}S | — | — | 8.0 | 7.0 | 5.0 |
| C₈-C₁₀ propyl | 2.0 | 2.0 | 2.0 | 2.0 | 1.0 |
| dimethyl amine | | | | | |
| C₁₂-C₁₄ alkyl dimethyl | — | — | — | — | 2.0 |
| amine oxide | | | | | |
| C₁₂-C₁₅AS | — | — | 17.0 | — | 8.0 |
| CFAA | — | 5.0 | 4.0 | 4.0 | 3.0 |
| C₁₂-C₁₄ Fatty alcohol | 12.0 | 6.0 | 1.0 | 1.0 | 1.0 |
| ethoxylate | | | | | |
| C₁₂-C₁₈ Fatty acid | 3.0 | — | 4.0 | 2.0 | 3.0 |
| Citric acid (anhydrous) | 4.5 | 5.0 | 3.0 | 2.0 | 1.0 |
| DETPMP | — | — | 1.0 | 1.0 | 0.5 |
| Monoethanolamine | 5.0 | 5.0 | 5.0 | 5.0 | 2.0 |
| Sodium hydroxide | — | — | 2.5 | 1.0 | 1.5 |
| 1N HCl aqueous solution | #1 | #1 | — | — | — |
| Propanediol | 12.7 | 14.5 | 13.1 | 10. | 8.0 |
| Ethanol | 1.8 | 2.4 | 4.7 | 5.4 | 1.0 |
| DTPA | 0.5 | 0.4 | 0.3 | 0.4 | 0.5 |
| Pectin Lyase | — | — | — | 0.005 | — |
| Amylase | 0.001 | 0.002 | — | | — |
| Cellulase | — | — | 0.0002 | | 0.0001 |
| Lipase | 0.1 | — | 0.1 | — | 0.1 |
| NprE (optional) | 0.05 | 0.3 | — | 0.5 | 0.2 |
| PMN | — | — | 0.08 | — | — |
| Protease A (optional) | — | — | — | — | 0.1 |
| Aldose Oxidase | — | — | 0.3 | — | 0.003 |
| ZnCl2 | 0.1 | 0.05 | 0.05 | 0.05 | 0.02 |
| Ca formate | 0.05 | 0.07 | 0.05 | 0.06 | 0.07 |
| DETBCHD | — | — | 0.02 | 0.01 | — |
| SRP1 | 0.5 | 0.5 | — | 0.3 | 0.3 |
| Boric acid | — | — | — | — | 2.4 |
| Sodium xylene sulfonate | — | — | 3.0 | — | — |
| Sodium cumene | — | — | — | 0.3 | 0.5 |
| sulfonate | | | | | |
| DC 3225C | 1.0 | 1.0 | 1.0 | 1.0 | 1.0 |
| 2-butyl-octanol | 0.03 | 0.04 | 0.04 | 0.03 | 0.03 |
| Brightener 1 | 0.12 | 0.10 | 0.18 | 0.08 | 0.10 |

Balance to 100% perfume/dye and/or water
#1: Add 1N HCl aq. soln to adjust the neat pH of the formula in the range from about 3 to about 5. The pH of Examples 16(I)-(II) is about 5 to about 7, and of 16(III)-(V) is about 7.5 to about 8.5.

### Example 17

### Liquid Hand Dishwashing Detergent Compositions Comprising Bsp Man4

In this example, various hand dish liquid detergent formulations are provided. In each of these formulations, Bsp Man4 is included at a concentration of from about 0.0001 to about 10 weight percent. In some alternative embodiments, other concentrations will find use, as determined by the formulator, based on their needs.

TABLE 17-1
Liquid Hand Dishwashing Detergent Compositions

| | Formulations | | | | | |
|---|---|---|---|---|---|---|
| Compound | I | II | III | IV | V | VI |
| C₁₂-C₁₅AE_{1.8}S | 30.0 | 28.0 | 25.0 | — | 15.0 | 10.0 |
| LAS | — | — | — | 5.0 | 15.0 | 12.0 |
| Paraffin Sulfonate | — | — | — | 20.0 | — | — |
| C₁₀-C₁₈ Alkyl | 5.0 | 3.0 | 7.0 | — | — | — |
| Dimethyl Amine | | | | | | |
| Oxide | | | | | | |
| Betaine | 3.0 | — | 1.0 | 3.0 | 1.0 | — |
| C₁₂ poly-OH fatty | — | — | — | 3.0 | — | 1.0 |
| acid amide | | | | | | |
| C₁₄ poly-OH fatty | — | 1.5 | — | — | — | — |
| acid amide | | | | | | |
| C₁₁E₉ | 2.0 | — | 4.0 | — | — | 20.0 |
| DTPA | — | — | — | — | 0.2 | — |
| Tri-sodium Citrate | 0.25 | — | — | 0.7 | — | — |
| dehydrate | | | | | | |
| Diamine | 1.0 | 5.0 | 7.0 | 1.0 | 5.0 | 7.0 |
| MgCl₂ | 0.25 | — | — | 1.0 | — | — |
| nprE (optional) | 0.02 | 0.01 | — | 0.01 | — | 0.05 |
| PMN | — | — | 0.03 | — | 0.02 | — |
| Protease A (optional) | — | 0.01 | — | — | — | — |
| Amylase | 0.001 | — | — | 0.002 | — | 0.001 |
| Aldose Oxidase | 0.03 | — | 0.02 | — | 0.05 | — |
| Sodium Cumene | — | — | — | 2.0 | 1.5 | 3.0 |
| Sulphonate | | | | | | |
| PAAC | 0.01 | 0.01 | 0.02 | — | — | — |
| DETBCHD | — | — | — | 0.01 | 0.02 | 0.01 |

Balance to 100% perfume/dye and/or water
The pH of Examples 17(I)-(VI) is about 8 to about 11.

### Example 18

### Liquid Automatic Dishwashing Detergent Compositions Comprising Bsp Man4

In this example, various liquid automatic dishwashing detergent formulations are provided. In each of these formulations, Bsp Man4 polypeptide is included at a concentration of from about 0.0001 to about 10 weight percent. In some alternative embodiments, other concentrations will find use, as determined by the formulator, based on their needs.

TABLE 18-1
Liquid Automatic Dishwashing Detergent Compositions

| | Formulations | | | | |
|---|---|---|---|---|---|
| Compound | I | II | III | IV | V |
| STPP | 16 | 16 | 18 | 16 | 16 |
| Potassium Sulfate | — | 10 | 8 | — | 10 |
| 1,2 propanediol | 6.0 | 0.5 | 2.0 | 6.0 | 0.5 |
| Boric Acid | — | — | — | 4.0 | 3.0 |
| CaCl₂ dihydrate | 0.04 | 0.04 | 0.04 | 0.04 | 0.04 |
| Nonionic | 0.5 | 0.5 | 0.5 | 0.5 | 0.5 |
| nprE (optional) | 0.1 | 0.03 | — | 0.03 | — |
| PMN | — | — | 0.05 | — | 0.06 |
| Protease B (optional) | — | — | — | 0.01 | — |
| Amylase | 0.02 | — | 0.02 | 0.02 | — |
| Aldose Oxidase | — | 0.15 | 0.02 | — | 0.01 |
| Galactose Oxidase | — | — | 0.01 | — | 0.01 |
| PAAC | 0.01 | — | — | 0.01 | — |
| DETBCHD | — | 0.01 | — | — | 0.01 |

Balance to 100% perfume/dye and/or water

### Example 19

### Granular and/or Tablet Laundry Compositions Comprising Bsp Man4

This example provides various formulations for granular and/or tablet laundry detergents. In each of these formulations, Bsp Man4 is included at a concentration of from about 0.0001 to about 10 weight percent. In some alternative embodiments, other concentrations will find use, as determined by the formulator, based on their needs.

TABLE 19-1
Granular and/or Tablet Laundry Compositions

| | Formulations | | | | |
|---|---|---|---|---|---|
| Compound | I | II | III | IV | V |
| Base Product | | | | | |
| C₁₄-C₁₅AS or TAS | 8.0 | 5.0 | 3.0 | 3.0 | 3.0 |
| LAS | 8.0 | — | 8.0 | — | 7.0 |
| C₁₂-C₁₅AE₃S | 0.5 | 2.0 | 1.0 | — | — |
| C₁₂-C₁₅E₅ or E₃ | 2.0 | — | 5.0 | 2.0 | 2.0 |
| QAS | — | — | — | 1.0 | 1.0 |
| Zeolite A | 20.0 | 18.0 | 11.0 | — | 10.0 |
| SKS-6 (dry add) | — | — | 9.0 | — | — |
| MA/AA | 2.0 | 2.0 | 2.0 | — | — |
| AA | — | — | — | — | 4.0 |
| 3Na Citrate 2H₂O | — | 2.0 | — | — | — |
| Citric Acid | 2.0 | — | 1.5 | 2.0 | — |
| (Anhydrous) | | | | | |
| DTPA | 0.2 | 0.2 | — | — | — |
| EDDS | — | — | 0.5 | 0.1 | — |
| HEDP | — | — | 0.2 | 0.1 | — |
| PB1 | 3.0 | 4.8 | — | — | 4.0 |
| Percarbonate | — | — | 3.8 | 5.2 | — |
| NOBS | 1.9 | — | — | — | — |
| NACA OBS | — | — | 2.0 | — | — |
| TAED | 0.5 | 2.0 | 2.0 | 5.0 | 1.00 |
| BB1 | 0.06 | — | 0.34 | — | 0.14 |
| BB2 | — | 0.14 | — | 0.20 | — |
| Anhydrous Na | 15.0 | 18.0 | — | 15.0 | 15.0 |
| Carbonate | | | | | |
| Sulfate | 5.0 | 12.0 | 5.0 | 17.0 | 3.0 |
| Silicate | — | 1.0 | — | — | 8.0 |
| nprE (optional) | 0.03 | — | 0.1 | 0.06 | — |
| PMN | — | 0.05 | — | — | 0.1 |
| Protease B (optional) | — | 0.01 | — | — | — |
| Protease C (optional) | — | — | — | 0.01 | — |
| Lipase | — | 0.008 | — | — | — |
| Amylase | 0.001 | — | — | — | 0.001 |
| Cellulase | — | 0.0014 | — | — | — |
| Pectin Lyase | 0.001 | 0.001 | 0.001 | 0.001 | 0.001 |
| Aldose Oxidase | 0.03 | — | 0.05 | — | — |
| PAAC | — | 0.01 | — | — | 0.05 |

Balance to 100% Moisture and/or Minors*
*Perfume, dye, brightener/SRP1/Na carboxymethylcellulose/photobleach/MgSO₄/PVPVI/suds suppressor/high molecular PEG/clay.

### Example 20

### Additional Liquid Laundry Detergents Comprising Bsp Man4

This example provides further formulations for liquid laundry detergents. In each of these formulations, Bsp Man4 is included at a concentration of from about 0.0001 to about 10 weight percent. In some alternative embodiments, other concentrations will find use, as determined by the formulator, based on their needs.

TABLE 20-1
Liquid Laundry Detergents

| | Formulations | | | | | |
|---|---|---|---|---|---|---|
| Compound | IA | IB | II | III | IV | V |
| LAS | 11.5 | 11.5 | 9.0 | — | 4.0 | — |
| C₁₂-C₁₅AE_{2.85}S | — | — | 3.0 | 18.0 | — | 16.0 |
| C₁₄-C₁₅E_{2.5}S | 11.5 | 11.5 | 3.0 | — | 16.0 | — |
| C₁₂-C₁₃E₉ | — | — | 3.0 | 2.0 | 2.0 | 1.0 |
| C₁₂-C₁₃E₇ | 3.2 | 3.2 | — | — | — | — |
| CFAA | — | — | — | 5.0 | — | 3.0 |
| TPKFA | 2.0 | 2.0 | — | 2.0 | 0.5 | 2.0 |
| Citric Acid (Anhy.) | 3.2 | 3.2 | 0.5 | 1.2 | 2.0 | 1.2 |
| Ca formate | 0.1 | 0.1 | 0.06 | 0.1 | — | — |
| Na formate | 0.5 | 0.5 | 0.06 | 0.1 | 0.05 | 0.05 |
| ZnCl2 | 0.1 | 0.05 | 0.06 | 0.03 | 0.05 | 0.05 |
| Na Culmene Sulfonate | 4.0 | 4.0 | 1.0 | 3.0 | 1.2 | — |
| Borate | 0.6 | 0.6 | 1.5 | — | — | — |
| Na Hydroxide | 6.0 | 6.0 | 2.0 | 3.5 | 4.0 | 3.0 |
| Ethanol | 2.0 | 2.0 | 1.0 | 4.0 | 4.0 | 3.0 |
| 1,2 Propanediol | 3.0 | 3.0 | 2.0 | 8.0 | 8.0 | 5.0 |
| Monoethanoaminel | 3.0 | 3.0 | 1.5 | 1.0 | 2.5 | 1.0 |
| TEPAE | 2.0 | 2.0 | — | 1.0 | 1.0 | 1.0 |
| nprE (optional) | 0.03 | 0.05 | — | 0.03 | — | 0.02 |
| PMN | — | — | 0.01 | — | 0.08 | — |
| Protease A (optional) | — | — | 0.01 | — | — | — |
| Lipase | — | — | — | 0.002 | — | — |
| Amylase | — | — | — | — | 0.002 | — |
| Cellulase | — | — | — | — | — | 0.0001 |
| Pectin Lyase | 0.005 | 0.005 | — | | — | — |
| Aldose Oxidase | 0.05 | — | — | 0.05 | — | 0.02 |
| Galactose oxidase | — | 0.04 | | | | |
| PAAC | 0.03 | 0.03 | 0.02 | — | — | — |
| DETBCHD | — | — | — | 0.02 | 0.01 | — |
| SRP 1 | 0.2 | 0.2 | — | 0.1 | — | — |
| DTPA | — | — | — | 0.3 | — | — |
| PVNO | — | — | — | 0.3 | — | 0.2 |
| Brightener 1 | 0.2 | 0.2 | 0.07 | 0.1 | — | — |
| Silicone antifoam | 0.04 | 0.04 | 0.02 | 0.1 | 0.1 | 0.1 |

Balance to 100% perfume/dye and/or water

### Example 21

### High Density Dishwashing Detergents Comprising Bsp Man4

This example provides various formulations for high density dishwashing detergents. In each of these compact formulations, Bsp Man4 is included at a concentration of from about 0.0001 to about 10 weight percent. In some alternative embodiments, other concentrations will find use, as determined by the formulator, based on their needs.

TABLE 21-1
High Density Dishwashing Detergents

| | Formulations | | | | | |
|---|---|---|---|---|---|---|
| Compound | I | II | III | IV | V | VI |
| STPP | — | 45.0 | 45.0 | — | — | 40.0 |
| 3Na Citrate | 17.0 | — | — | 50.0 | 40.2 | — |
| 2H₂O | | | | | | |
| Na Carbonate | 17.5 | 14.0 | 20.0 | — | 8.0 | 33.6 |
| Bicarbonate | — | — | — | 26.0 | — | — |
| Silicate | 15.0 | 15.0 | 8.0 | — | 25.0 | 3.6 |
| Metasilicate | 2.5 | 4.5 | 4.5 | — | — | — |
| PB1 | — | — | 4.5 | — | — | — |
| PB4 | — | — | — | 5.0 | — | — |
| Percarbonate | — | — | — | — | — | 4.8 |
| BB1 | — | 0.1 | 0.1 | — | 0.5 | — |
| BB2 | 0.2 | 0.05 | — | 0.1 | — | 0.6 |
| Nonionic | 2.0 | 1.5 | 1.5 | 3.0 | 1.9 | 5.9 |
| HEDP | 1.0 | — | — | — | — | — |
| DETPMP | 0.6 | — | — | — | — | — |
| PAAC | 0.03 | 0.05 | 0.02 | — | — | — |
| Paraffin | 0.5 | 0.4 | 0.4 | 0.6 | — | — |
| nprE (optional) | 0.072 | 0.053 | — | 0.026 | — | 0.01 |
| PMN | — | — | 0.053 | — | 0.059 | — |
| Protease B | — | — | — | — | — | 0.01 |
| (optional) | | | | | | |
| Amylase | 0.012 | — | 0.012 | — | 0.021 | 0.006 |
| Lipase | — | 0.001 | — | 0.005 | — | — |
| Pectin Lyase | 0.001 | 0.001 | 0.001 | — | — | — |
| Aldose Oxidase | 0.05 | 0.05 | 0.03 | 0.01 | 0.02 | 0.01 |
| BTA | 0.3 | 0.2 | 0.2 | 0.3 | 0.3 | 0.3 |
| Polycarboxy- | 6.0 | — | — | — | 4.0 | 0.9 |
| late | | | | | | |
| Perfume | 0.2 | 0.1 | 0.1 | 0.2 | 0.2 | 0.2 |

Balance to 100% Moisture and/or Minors*
*Brightener/dye/SRP1/Na carboxymethylcellulose/photobleach/MgSO₄/PVPVI/suds suppressor/high molecular PEG/clay. The pH of Examples 21(I) through (VI) is from about 9.6 to about 11.3.

### Example 22

### Tablet Dishwashing Detergent Compositions Comprising Bsp Man4

This example provides various tablet dishwashing detergent formulations. The following tablet detergent compositions of the present disclosure are prepared by compression of a granular dishwashing detergent composition at a pressure of 13KN/cm² using a standard 12 head rotary press. In each of these formulations, Bsp Man4 is included at a concentration of from about 0.0001 to about 10 weight percent. In some alternative embodiments, other concentrations will find use, as determined by the formulator, based on their needs.

TABLE 22-1
Tablet Dishwashing Detergent Compositions

| | Formulations | | | | | | | |
|---|---|---|---|---|---|---|---|---|
| Compound | I | II | III | IV | V | VI | VII | VIII |
| STPP | — | 48.8 | 44.7 | 38.2 | — | 42.4 | 46.1 | 46.0 |
| 3Na Citrate 2H₂O | 20.0 | — | — | — | 35.9 | — | — | — |
| Na Carbonate | 20.0 | 5.0 | 14.0 | 15.4 | 8.0 | 23.0 | 20.0 | — |
| Silicate | 15.0 | 14.8 | 15.0 | 12.6 | 23.4 | 2.9 | 4.3 | 4.2 |
| Lipase | 0.001 | — | 0.01 | — | 0.02 | — | — | — |
| Protease B | 0.01 | — | — | — | — | — | — | — |
| (optional) | | | | | | | | |
| Protease C | — | — | — | — | — | 0.01 | — | — |
| (optional) | | | | | | | | |
| nprE (optional) | 0.01 | 0.08 | — | 0.04 | — | 0.023 | — | 0.05 |
| PMN | — | — | 0.05 | — | 0.052 | — | 0.023 | — |
| Amylase | 0.012 | 0.012 | 0.012 | — | 0.015 | — | 0.017 | 0.002 |
| Pectin Lyase | 0.005 | — | — | 0.002 | — | — | — | — |
| Aldose Oxidase | — | 0.03 | — | 0.02 | 0.02 | — | 0.03 | — |
| PB1 | — | — | 3.8 | — | 7.8 | — | — | 4.5 |
| Percarbonate | 6.0 | — | — | 6.0 | — | 5.0 | — | — |
| BB1 | 0.2 | — | 0.5 | — | 0.3 | 0.2 | — | — |
| BB2 | — | 0.2 | — | 0.5 | — | — | 0.1 | 0.2 |
| Nonionic | 1.5 | 2.0 | 2.0 | 2.2 | 1.0 | 4.2 | 4.0 | 6.5 |
| PAAC | 0.01 | 0.01 | 0.02 | — | — | — | — | — |
| DETBCHD | — | — | — | 0.02 | 0.02 | — | — | — |
| TAED | — | — | — | — | — | 2.1 | — | 1.6 |
| HEDP | 1.0 | — | — | 0.9 | — | 0.4 | 0.2 | — |
| DETPMP | 0.7 | — | — | — | — | — | — | — |
| Paraffin | 0.4 | 0.5 | 0.5 | 0.5 | — | — | 0.5 | — |
| BTA | 0.2 | 0.3 | 0.3 | 0.3 | 0.3 | 0.3 | 0.3 | — |
| Polycarboxylate | 4.0 | — | — | — | 4.9 | 0.6 | 0.8 | — |
| PEG 400-30,000 | — | — | — | — | — | 2.0 | — | 2.0 |
| Glycerol | — | — | — | — | — | 0.4 | — | 0.5 |
| Perfume | — | — | — | 0.05 | 0.2 | 0.2 | 0.2 | 0.2 |

Balance to 100% Moisture and/or Minors*
*Brightener/SRP1/Na carboxymethylcellulose/photobleach/MgSO₄/PVPVI/suds suppressor/high molecular PEG/clay. The pH of Examples 22(I) through 22(VII) is from about 10 to about 11.5; pH of 22(VIII) is from 8-10. The tablet weight of Examples 22(I) through 22(VIII) is from about 20 grams to about 30 grams.

### Example 23

### Liquid Hard Surface Cleaning Detergents Comprising Bsp Man4

This example provides various formulations for liquid hard surface cleaning detergents. In each of these formulations, Bsp Man4 is included at a concentration of from about 0.0001 to about 10 weight percent. In some alternative embodiments, other concentrations will find use, as determined by the formulator, based on their needs.

TABLE 23-1
Liquid Hard Surface Cleaning Detergents

| | Formulations | | | | | | |
|---|---|---|---|---|---|---|---|
| Compound | I | II | III | IV | V | VI | VII |
| C₉-C₁₁E₅ | 2.4 | 1.9 | 2.5 | 2.5 | 2.5 | 2.4 | 2.5 |
| C₁₂-C₁₄E₅ | 3.6 | 2.9 | 2.5 | 2.5 | 2.5 | 3.6 | 2.5 |
| C₇-C₉E₆ | — | — | — | — | 8.0 | — | — |
| C₁₂-C₁₄E₂₁ | 1.0 | 0.8 | 4.0 | 2.0 | 2.0 | 1.0 | 2.0 |
| LAS | — | — | — | 0.8 | 0.8 | — | 0.8 |
| Sodium culmene | 1.5 | 2.6 | — | 1.5 | 1.5 | 1.5 | 1.5 |
| sulfonate | | | | | | | |
| Isachem ® AS | 0.6 | 0.6 | — | — | — | 0.6 | — |
| Na₂CO₃ | 0.6 | 0.13 | 0.6 | 0.1 | 0.2 | 0.6 | 0.2 |
| 3Na Citrate 2H₂O | 0.5 | 0.56 | 0.5 | 0.6 | 0.75 | 0.5 | 0.75 |
| NaOH | 0.3 | 0.33 | 0.3 | 0.3 | 0.5 | 0.3 | 0.5 |
| Fatty Acid | 0.6 | 0.13 | 0.6 | 0.1 | 0.4 | 0.6 | 0.4 |
| 2-butyl octanol | 0.3 | 0.3 | — | 0.3 | 0.3 | 0.3 | 0.3 |
| PEG DME-2000 ® | 0.4 | — | 0.3 | 0.35 | 0.5 | — | — |
| PVP | 0.3 | 0.4 | 0.6 | 0.3 | 0.5 | — | — |
| MME PEG (2000) ® | — | — | — | — | — | 0.5 | 0.5 |
| Jeffamine ® ED-2001 | — | 0.4 | — | — | 0.5 | — | — |
| PAAC | — | — | — | 0.03 | 0.03 | 0.03 | — |
| DETBCHD | 0.03 | 0.05 | 0.05 | — | — | — | — |
| nprE (optional) | 0.07 | — | 0.08 | 0.03 | — | 0.01 | 0.04 |
| PMN | — | 0.05 | — | — | 0.06 | — | — |
| Protease B (optional) | — | — | — | — | — | 0.01 | — |
| Amylase | 0.12 | 0.01 | 0.01 | — | 0.02 | — | 0.01 |
| Lipase | — | 0.001 | — | 0.005 | — | 0.005 | — |
| Pectin Lyase | 0.001 | — | 0.001 | — | — | — | 0.002 |
| ZnCl2 | 0.02 | 0.01 | 0.03 | 0.05 | 0.1 | 0.05 | 0.02 |
| Calcium Formate | 0.03 | 0.03 | 0.01 | — | — | — | — |
| PB1 | — | 4.6 | — | 3.8 | — | — | — |
| Aldose Oxidase | 0.05 | — | 0.03 | — | 0.02 | 0.02 | 0.05 |

Balance to 100% perfume/dye and/or water
The pH of Examples 23(I) through (VII) is from about 7.4 to about 9.5.

1. A recombinant polypeptide comprising a catalytic domain of an endo-β-mannanase, wherein the catalytic domain is at least 85% identical to the amino acid sequence of SEQ ID NO:9 or a mature form of an endo-β-mannanase, wherein the mature form is at least 80% identical to the amino acid sequence of SEQ ID NO:8.

2. (canceled)

3. The recombinant polypeptide of claim 1, wherein the polypeptide has mannanase activity in the presence of detergent.

4. The recombinant polypeptide of claim 1, wherein the polypeptide has mannanase activity in the presence of a protease.

5. The recombinant polypeptide of claim 1, wherein the polypeptide retains greater than 70% mannanase activity at pH values of between 6 and 8.5.

6. The recombinant polypeptide of claim 1, wherein the polypeptide retains greater than 70% mannanase activity at a temperature range from 55° C. to 65° C.

7. The recombinant polypeptide of claim 1, wherein the polypeptide is capable of hydrolyzing a substrate selected from the group consisting of chocolate ice cream, guar gum, locust bean gum, and combinations thereof.

8. The recombinant polypeptide of claim 1, wherein the amino acid sequence is at least 95% identical to one of the group consisting of SEQ ID NOS:6-14 and 30-49.

9. (canceled)

10. The recombinant polypeptide of claim 1, further comprising a native or non-native signal peptide.

11. The recombinant polypeptide of claim 1, wherein the polypeptide does not further comprise a carbohydrate-binding module.

12. A detergent composition comprising the recombinant polypeptide of claim 1.

13. The detergent composition of claim 12, further comprising a surfactant.

14. The detergent composition of claim 13, wherein the surfactant is an ionic surfactant.

15. The detergent composition of claim 13, wherein the surfactant is selected from the group consisting of an anionic surfactant, a cationic surfactant, a zwitterionic surfactant, and a combination thereof.

16. The detergent composition of claim 12, further comprising an enzyme selected from the group consisting of proteases, peroxidases, cellulases, beta-glucanases, hemicellulases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-β-mannanases, exo-β-mannanases, pectin methylesterases, cellobiohydrolases, transglutaminases, and combinations thereof.

17. (canceled)

18. The detergent composition of claim 12, wherein the detergent is selected from the group consisting of a laundry detergent, a fabric softening detergent, a dishwashing detergent, and a hard-surface cleaning detergent.

19. The detergent composition of claim 12, wherein the detergent is in a form selected from the group consisting of a liquid, a powder, a granulated solid, and a tablet.

20. A method for hydrolyzing a mannan substrate present in a soil or stain on a surface, comprising: contacting the surface with the detergent composition of claim 12 to produce a clean surface.

21. A method of textile cleaning comprising: contacting a soiled textile with the detergent composition of claim 12 to produce a clean textile.

22-39. (canceled)

40. The detergent composition of claim 13, wherein the surfactant is a non-ionic surfactant.


SEQUENCE LISTING (49 sequences)

SEQ ID NO: 1 - DNA, 3273 bp, Bacillus sp. SWT81
atgggaacatggaaaaaggggtttgtgttatttattgtcctaatgttagtttttgatgtatcgatgttgggtgtaaatgtaagcgcttcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaagaagaaacaattcgtttatttgtaaaaattcctgaaatgtctttgttaacattaacgtttgatgatgatataaacggaatcaaaagcaatggaacatggcctgaagatggtgtaacatctgaaattgaccacgctattgtagatggagacggcaagttgatgttctctgttcaaggaatgtcacctactgaaacatggcaagagctcaagttagaattaacagaactatcagatgtgaacattgatgcggttaagaaaatgaagtttgacgcgcttatcccagcaggtagtgaagaaggttcagtccaaggaatcgtacaacttccaccggattgggagacgaaatatgggatgaatgaaacaacgaagtcaataaaagacttagagactgttactgttaatggaagcgattataaacggttggaagtgactgtttctatcgacaatcaaggaggagctacaggaatcgctttatcattagtaggatcccaactcgatttgttagaacctgtctacatcgataatattgaacttctaaattcctttgaagcaccaccagcagattcttttcttgttgatgattttgaaggttattttggggatgacacgttgttacatcgcaattattctagcaatggagatccaattacactatcgttaacaagtgagtttaaaaataatggagaatttggattgaagtatgattattcgattggctcgatgggttatgcagggaggcaaacatcactaggacctgtcgattggagcggagctaatgcttttgaattttggatgaaacatggacaacttgaagggaatcatttaactgtacaaattcgaataggtgatgttagctttgaaaaaaatcttgaattaatggatgctcatgaaggtgtagtgacaatcccgttttctgaatttgctccagctgcttgggaaaataagcctggcgttatcattgacgaacaaaaattgaaaagagtgagtcaatttgctctttacacaggcggggctagacaatctggaacaatctactttgatgatttacgagcggtatatgatgaaagtttaccatcagttccagttccgaaagaggaggaagaggaaaaagaggtcgctcctattatttatcattttgaatctggaattgataattgggaagggggacaagcaacacatagcaatgggcacctcaaagtaacggttcgtttaggtgaaggtcagcaaaccgaagtgaagaaaacatcaaattataatttaacagggtataattatatagtagctaatataaaacatgacgatacaggaatgtttggtagtgacccgcttcaagtgaaaatctttacgaaagcaggaggttgggtatgggctgattcaggaaatcaaccgatttactccgacgattatactcaagttgtgtatgatattactactttagctaacaaaaatgcagtccaagaaatcgggtttgaatttttggctccttcaggttcttcagggacgacgaatcctttcatagattcagtagcgattgttacgagtctcgatcaattgtctgagcagccagagcagccagaacaaccaggaacaccagatactgatgataataaagaggataaagatagaagaaatgtagaagtgaacgaggaaggacaaaaactacccaaaacagcaacgtcaatatttaattatttgctaattggttttgtttttgtagggattggatttagtctatttatttataaaagaagaaaaacagtg

SEQ ID NO: 2 - protein, 1091 aa, Bacillus sp. SWT81
MGTWKKGFVLFIVLMLVFDVSMLGVNVSASQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKEVAPIIYHFESGIDNWEGGQATHSNGHLKVTVRLGEGQQTEVKKTSNYNLTGYNYIVANIKHDDTGMFGSDPLQVKIFTKAGGWVWADSGNQPIYSDDYTQVVYDITTLANKNAVQEIGFEFLAPSGSSGTTNPFIDSVAIVTSLDQLSEQPEQPEQPGTPDTDDNKEDKDRRNVEVNEEGQKLPKTATSIFNYLLIGFVFVGIGFSLFIYKRRKTV

SEQ ID NO: 3 - DNA, 43 bp, Artificial Sequence
  synthetic primer
tgagcgcgcaggcagctggtaaatcacaagaagggcgtcaact

SEQ ID NO: 4 - DNA, 32 bp, Artificial Sequence
  synthetic primer
cgcctcgagttacactgtttttcttcttttat

SEQ ID NO: 5 - DNA, 3282 bp, Artificial Sequence
  synthetic BSP Man4 gene
gtgagaagcaaaaaattgtggatcagcttgttgtttgcgttaacgttaatctttacgatggcgttcagcaacatgagcgcgcaggcagctggtaaatcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaagaagaaacaattcgtttatttgtaaaaattcctgaaatgtctttgttaacattaacgtttgatgatgatataaacggaatcaaaagcaatggaacatggcctgaagatggtgtaacatctgaaattgaccacgctattgtagatggagacggcaagttgatgttctctgttcaaggaatgtcacctactgaaacatggcaagagctcaagttagaattaacagaactatcagatgtgaacattgatgcggttaagaaaatgaagtttgacgcgcttatcccagcaggtagtgaagaaggttcagtccaaggaatcgtacaacttccaccggattgggagacgaaatatgggatgaatgaaacaacgaagtcaataaaagacttagagactgttactgttaatggaagcgattataaacggttggaagtgactgtttctatcgacaatcaaggaggagctacaggaatcgctttatcattagtaggatcccaactcgatttgttagaacctgtctacatcgataatattgaacttctaaattcctttgaagcaccaccagcagattcttttcttgttgatgattttgaaggttattttggggatgacacgttgttacatcgcaattattctagcaatggagatccaattacactatcgttaacaagtgagtttaaaaataatggagaatttggattgaagtatgattattcgattggctcgatgggttatgcagggaggcaaacatcactaggacctgtcgattggagcggagctaatgcttttgaattttggatgaaacatggacaacttgaagggaatcatttaactgtacaaattcgaataggtgatgttagctttgaaaaaaatcttgaattaatggatgctcatgaaggtgtagtgacaatcccgttttctgaatttgctccagctgcttgggaaaataagcctggcgttatcattgacgaacaaaaattgaaaagagtgagtcaatttgctctttacacaggcggggctagacaatctggaacaatctactttgatgatttacgagcggtatatgatgaaagtttaccatcagttccagttccgaaagaggaggaagaggaaaaagaggtcgctcctattatttatcattttgaatctggaattgataattgggaagggggacaagcaacacatagcaatgggcacctcaaagtaacggttcgtttaggtgaaggtcagcaaaccgaagtgaagaaaacatcaaattataatttaacagggtataattatatagtagctaatataaaacatgacgatacaggaatgtttggtagtgacccgcttcaagtgaaaatctttacgaaagcaggaggttgggtatgggctgattcaggaaatcaaccgatttactccgacgattatactcaagttgtgtatgatattactactttagctaacaaaaatgcagtccaagaaatcgggtttgaatttttggctccttcaggttcttcagggacgacgaatcctttcatagattcagtagcgattgttacgagtctcgatcaattgtctgagcagccagagcagccagaacaaccaggaacaccagatactgatgataataaagaggataaagatagaagaaatgtagaagtgaacgaggaaggacaaaaactacccaaaacagcaacgtcaatatttaattatttgctaattggttttgtttttgtagggattggatttagtctatttatttataaaagaagaaaaacagtg

SEQ ID NO: 6 - protein, 1094 aa, Artificial Sequence
  synthetic Bsp Man4 sequence
MRSKKLWISLLFALTLIFTMAFSNMSAQAAGKSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKEVAPIIYHFESGIDNWEGGQATHSNGHLKVTVRLGEGQQTEVKKTSNYNLTGYNYIVANIKHDDTGMFGSDPLQVKIFTKAGGWVWADSGNQPIYSDDYTQVVYDITTLANKNAVQEIGFEFLAPSGSSGTTNPFIDSVAIVTSLDQLSEQPEQPEQPGTPDTDDNKEDKDRRNVEVNEEGQKLPKTATSIFNYLLIGFVFVGIGFSLFIYKRRKTV

SEQ ID NO: 7 - protein, 1065 aa, Artificial Sequence
  synthetic Bsp Man4 sequence
AGKSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKEVAPIIYHFESGIDNWEGGQATHSNGHLKVTVRLGEGQQTEVKKTSNYNLTGYNYIVANIKHDDTGMFGSDPLQVKIFTKAGGWVWADSGNQPIYSDDYTQVVYDITTLANKNAVQEIGFEFLAPSGSSGTTNPFIDSVAIVTSLDQLSEQPEQPEQPGTPDTDDNKEDKDRRNVEVNEEGQKLPKTATSIFNYLLIGFVFVGIGFSLFIYKRRKTV

SEQ ID NO: 8 - protein, 1062 aa, Bacillus sp. SWT81
SQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKEVAPIIYHFESGIDNWEGGQATHSNGHLKVTVRLGEGQQTEVKKTSNYNLTGYNYIVANIKHDDTGMFGSDPLQVKIFTKAGGWVWADSGNQPIYSDDYTQVVYDITTLANKNAVQEIGFEFLAPSGSSGTTNPFIDSVAIVTSLDQLSEQPEQPEQPGTPDTDDNKEDKDRRNVEVNEEGQKLPKTATSIFNYLLIGFVFVGIGFSLFIYKRRKTV

SEQ ID NO: 9 - protein, 296 aa, Bacillus sp. SWT81
DEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDW

SEQ ID NO: 10 - protein, 469 aa, Bacillus sp. SWT81
RQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEK

SEQ ID NO: 11 - protein, 383 aa, Bacillus sp. SWT81
RQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTV

SEQ ID NO: 12 - protein, 849 aa, Artificial Sequence
  synthetic Bsp Man4 sequence
AGKSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKE

SEQ ID NO: 13 - protein, 669 aa, Artificial Sequence
  synthetic Bsp Man4 sequence
AGKSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFL

SEQ ID NO: 14 - protein, 492 aa, Artificial Sequence
  synthetic Bsp Man4 sequence
AGKSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMS

SEQ ID NO: 15 - protein, 556 aa, Bacillus halodurans
ASGQELKMTDQNASQYTKELFAFLRDVSGKQVLFGQQHATDEGLTLRGTGNRIGSTESEVKNAVGDYPAVFGWDTNSLDGREKPGNDEPSQEQRILNTAASMKAAHDLGGIITLSMHPDNFVTGGAYGDTTGNVVQEILPGGSKHEEFNAWLDNLAALAHELKDDNGKHIPIIFRPFHEQTGSWFWWGASTTTPEQYKAIYRYTVEYLRDVKGANNFLYGFSPGAGPAGDLNRYMETYPGDDYVDIFGIDNYDNKSNAGSEAWIQGVVTDLAMLVDLAEEKGKIAAFTEYGYSATGMNRTGNTLDWYTRLLNAIKEDPKASKISYMLTWANFGFPNNMYVPYKDIHGDLGGDHELLPDFIKFFEDDYSAFTGDIKGNVYDTGIEYTVAPHERLMYVLSPITGTTITDTVTLRAKVLNDDNAVVTYRVEGSDVEHEMTLADSGYYTAKYSPTAEVNGGSVDLTVTYWSGEEKVQDEVIRLYVKASEISLYKLTFDEDINGIKSNGTWPEDGITSDVSHVSFDGNGKLKFAVNGMSSEEWWQELKLELTDLSDVNLAK

SEQ ID NO: 16 - protein, 1066 aa, Bacillus cellulosilyticus
EETRVLKMSNPDASKYTKELFAYLQDVGSDNVLFGQQHATDEGLTIKVENGDSNFVGSTQSEVKNAVGDYPAVFGWDTNSLDGRERPGNPISGEPLTQEQRTQNLAKSMITAHELGGIITLSMHPDNFVTGEYYGDTDGNVVKTILPGGVHHDDYNEWLDNIVDLSHLVVDEDGHHIPIIFRPFHEQNGSWFWWGASTTTPEQYKAIFRYTVEYLREHGANNFLIGFSPNGASAGDLEQYLETYPGDDYVDILGIDRYDTKSNAGSQEWLTAVAKDLAMISKEAEDRGKISAFTEFGYSPTGMNEEGNNLQWWEDLLSAIMDNPDYPEAANIAFMSTWANFGFPNNMYVPYRDIHGDLGGDHELLPTFEEFYNDESTLFSEEVKGQIYHSGKTLETAEHDSKMYTLSPTDGDTITENKVTLLTRVVNDDDATVTYSVDGSEEVEMELAGRYYTADWIPNALQNGGTANITIRHYDGNNNEVSKEVIRTYLRVPEILVEEITFDDSIEGALNKGTWPEVGVEFELSHEKLGGDGKLALSVSGMPEDEWWQELKIGFEDLSHVNFDVVNQVKFDVLLPETVADGAILSTVLAADGNTKYGEGTTERNVTDLEIIEIDGVEYKLYETTINLEESITEGTELGIIGKQLDFSNKLYLDNVRFLNAYLEAPTDPLLVDDFEGYLGDNDLLNRNYSNPGDRILISLSSEHKHSGEYGLQYDWTIGSSGYAGRQTSLGPVDWSGTNAFQFWLKHDDLPDNSLTVQIQMGGVSFEASTDLDESFEGIVTIPFVDFAPAHWEGNQTAIIDKPRLERVSQFALYMGGNEGSGTLYFDDLRAVYDEDAPPVPEREDAGEIEPIIYDFESDLDGWGTNMSLIKDGNLVHPVGLGEGNKTEIAKTSGYDLSGHNYIVATVKHDEEGTFGDDPLNAKLFIKTGSAWTWADSGDFSLNSDKYVEIVFDISDNAARENVQEIGLEFTAPAGSDGTSNAYIESIKILTALEELPDTEDPGETDEVQELKDLISDLKERIKELENNTDVEDFDKRVQELTNELNHLKAKYNDMEKLVPVIEQRL

SEQ ID NO: 17 - protein, 510 aa, Cellulomonas fimi
MPSAQAQEQIINLVDAEASTSTKQLFSYLQSISGEKVLFGQQHATDEGITVTGPGLRTGSTESEVKNSVGDYPALFGWDTLSLDGYEKPGSREQSAAENRANLIKSMKTAHELGGILTLSTHPHNFVTGGDFYDTSGRVVKNILPGGSYNARFNEWLDNIAAFANDLKDNEGKDIPVIFRPFHEQTGGWFWWGAQTTSAAEYKELYRYTVEYLRDVKGVDNFLYAFSPGASFNGDEEKYLKTYPGDDYVDVLGFDQYDNPNNPGSEGFLNTLVVDLGMLSKLADSKGKIAALTEYGLGLKTNGNLDTQWFTRVLDAIKADPYARKISYMQTWANFGLNGNLFVPYKNAPNGLGDHELLPDFINFYKDPYSAFSKDVGNIYRGAVPETVAEKPFMHIVSPIDRSLSLQKVTPISVSVIQGKPKDIYYTVNDKAKKYPLVKGDGYYYEGSAALKGDKATIHVTAEFADGTSQKQTIKVYLKEPEKQPPTVVDTFETYYGDDEQLQAAFATQG

SEQ ID NO: 18 - protein, 964 aa, Bacillus sp.
SEGQSFKLVDSNASTLTKSLYAYLQDTSGRQILFGHQHAVDEGLTLTNSGDRVGSTQSEVKNAVGDYPAIFGWDTLSLDGYEKPGNEKNSQAQNRANVVQSMRTVHELGGIIALSMHPENFVTGNQYNDTSGDVVKNILPDGSHHEVFNAWLDNIAAFAHELTDQSTGELIPVIFRPFHEQNGGWFWWGAQTTTASEYKALYRYTVDYLRDVKGVNNFLYAFSPNAPFDGNLTQYLRTYPGDQYVDIFGLDQYDNKANAGQATFLNGLTQDLAMISKLADEKGKIAAFTEYGYSPQGFNETGNYLQWYTAVLEAIKKDPNASRIAYMQTWANFGYPTNMFVPYRDVNGNLGGDHELLPNFVEFYEDDYAAFLTEASGWNLYQDISTIEQEPFMHIVTPTANSQISEAVTIRARVLHDQPSHVVFEVNDSGEEIPMSLDEDGFFYMGKWTPDAAVNHTTVNITVRAYGENQVQEETFPLVVRVSEMLLKEYTFDEGIEGIQNNGTYPDTIETSFEHQVLNGDGKLKINVAGLQASDTWQELKLELTNLHDVQLGNVNRVKVDVFIPKAAVNQSATIRGIVQLPPDWDTKYGMTTTEKNLSDLQSVVIDEEEYVEGQITIDLTSPEASAAATGLALSLVGNAIDFTGAIYVDNIQLIGVSEEEVSDPAIVDDFESYVGNDDLLRNAWVAANGGIAISLDQEEKSAGDYGLAYEYSLAGAGSYTGITKMLGNRDWSSYNSLQFWMNSDGNGQKLVIQAEIGGVHFEAYPSLEANEEGLVTIGFNEFTPAPWESASNLEKLVTEEALKNVTKLSLYINAQDELDSALVSTLFFDEIRAAYVEEEPGEEGEPGEEGKSGEEGKPGEEGEPGEEGEPGEEGKPGEEGELGEEGKPGEEGELGEEEEPGEEGELGEELEVGHKEQGNQSSSGANKLPSTATNVFNFLLIGTLLVIGSTSLLYMRRKKINNE

SEQ ID NO: 19 - protein, 829 aa, Enterococcus faecium
SYADSYNMVDERASEKTRQLFGFLQATQDSSGIMFGHQHALDEGVTLTGEAPRTGSTDSEVKNAVGDYPAVFGWDTGSLDGGEKPGVAGDVEQSIQNTAISMKTAYDLGGVIVLSMHPRNFVTGGAYNDLTGNVVQNILPGGDYNDTFNAWLDQIATLSYLLKDDDGNSIPFIFRPFHEQTGSWFWWGESTTTTEQYKAIYRYTVDYLKNTKDVHNILYAYTPNKMTPGDEERYMRTYPGDDYVDIFGIDIYDQQENAGSEEFLDSVVQDLSMITSIAESKNKIAALSEFGYSAVGLKETGNTLDWYTRLFNAIKNNTQASKIAYMLTWANFGLPTNLYVPYRDVNGDLGGDHEMLPDFVSYYDDPASLFLEEVKGNIYIPENSGETVQQSAQFFVLNPTNKLTITNSELPIYTMATNDDSAEVTYEIEGITEETALTRQGNLFGGSFDLGDNFKNGSLNLVLRYYSAGVEVESENIQVYMQAEINESELLIDDFESYLGEDELLNQKYVSSGDPITMSLSEVKNSGDYGLKFDYTIASQGYSGRQLSVEKDWSNATGISFWMANEVASQDTLTIQIRIGSVSFEAYVDLSSPYTGIVEIPFDEFVPASWEQNQSAEINSETLQSVSLFALYMGGSKGEGTLYFDDIQAVDASSVVDPTEYTVTVIHEDVDGNILLTEDIQAEEGKTVTVESKQFDGYLIQGDATQEILVDGNQEVIFLYSKVATDTTDTTDTTDTTDTTDTTDTTDTTDTTDTTDTTDTTETSEATENSGNKSKVAVVNNNSNTTGGSTNSSGKALPQTGEKNQLAISILGVAVIVAVVGAVLYKKKA

SEQ ID NO: 20 - protein, 475 aa, Cellulomonas fimi
MADETIAIVDADATAETRSLLSYLDGVRGEGILFGHQHTTSFGLTTGPTDGTTSDVKNVTGDFPAVFGWDTLIIEGNERPGLAENTRDENIALFADYIRKADAIGGVNTVSAHVENFVTGGSFYDTSGDTLRAVLPGGSHHAELVAYLDDIAELADASRRDDGTLIPIVFRPWHENAGSWFWWGAAYGSPGEYQELYRFTVEYLRDVKGVSNFLYAWGPGGGFGGNRDVYLRTYPGDAFVDVLGLDTYDSTGSDAFLAGLVADLRMIAEIADEKGKVSAFTEFGVSGGVGTNGSSPAQWFTKVLAAIKADPVASRNAYMETWANFDAGQHFVPVPGDALLEDFQAYAADPFTLFASEVTGAFDRTVAAAPAQPVVHIASPADGARVASAPTTVRVRVGGTDVQSVTVEVAQGGTVVDTLDLAYDGALWWTAPWSPTSAQLDNSTYTVTATATTAAGTLDVTNEVAAALEHHHHHH

SEQ ID NO: 21 - protein, 1008 aa, Geobacillus tepidamans
KKQKNPSKPNSKRVENLVDPLATDDTKSLFAYLKDVRGKQVLFGHQHAIDEGLTLIGSKELESEVKNSVGDFPAVFGWDTLSLEGKEKPGVPNDPKQSRANLVASMKKVHKLGGIIALSAHMPNFVTGGSFNDTTGNVVEHILPGGDKNAEFNSFLDNIAQFAKELKDDKGKQIPILFRPFHEQNGSWFWWGAKTTTPSQYIEIYRYTVEYLRDKKGVHNFLYVYSPNGTFGGSEANYLTTYPGDDYVDILGMDQYDNQSNPGTTQFLTNLVKDLEMISKLADTKGKIAAFSEFGYSPQGMKTTGNGDLKWFTKVLNAIKADRNAKRIAYMQTWANFGLNGNLFVPYNDAPNGLGDHELLPDFINYYKDPYTAFLREVKGVYNNKVEAAKEQPFMHIASPTDNATVKTATTKIRVRVLNQKPSKVVYVVEGSSKEVPMKLDADGYYSANWSPVSKFNGKSVKITVKSYMPNKTVMKQTVNVFVKVPEILIKQFTFDRDIKGIRNIGTWPDTIKTNFEHARLNGNGKLKINITGMVRTDTWQEIKLELSNIKDIVPLSNVNRVKFDVLVPVSAGQQNANASLRGIIMLPPDWNEKYGMTTTEKALANLQTVTINRVKYAEFPVMIDLNDPAKLSAAKGLVLSIVGNGLELNGAVYVDNIKLFSTYTETPTDPALVDDFESYQGSNAVLQQKFVKAGGDTITVSLDGSHKSSGTYAMKVDYTLAGSGYAGVTKSLGGVDWSRFNKLKFWLTPDGKDQKLVIQLRVDGVYYEAYPSLASTTPGWVELHFNDFTVAPWDTANLGKKLNKISLKNVQDFAIYVNSKNGTTLSSTLYFDDIKAIYDATAASVPNGGTGPGSTPEQPGTLYDFETGVQGWEVEQNQANATTPTITTDAAAKGTHSLTSTFDLTKTGGFELTKVQVVDLSAVKTISAKVKISTGTANARLYIKTGSNWQWHDSGMVAVDSSEFKTLTISLNPAWGIDNVKSIGVKIEPTSGTGNASVYVDDVALSE

SEQ ID NO: 22 - protein, 1010 aa, Thermoanaerobacterium thermosaccharolyticum
NNSQNNSNNGSTIKEINLVDPNATTETKELFVYLNDIRGKEVLFGHQHDTDEGITITSGSNELQSDVKNDVGDFPAVFGWDTLSLEGKEKPGVPNDPVKSRENLIAAVKKIHEMGGIFTLSAHMPNFVTGGSFNDVSDDVVDKILPGGEYNSKFNEFLDNIALFANNLKDDNGNLIPILFRPFHEQNGGWFWWGAKTTTPSQYIELYRYTVEYLRDKKGVHNILYVYSPNGPFNGNEENYLVTYPGDIYVDVLGMDQYDNIDNPGTKQFLSSLVNDLSMISKLADSKGKIATLSEFGYSPQGMKVTGNGDLSWFTDVLNAIKSNSNARRIAYMLTWANFGLNGNLFVPYKNAPNLGDHELLPDFIKFYQDPYTAFLNDIKGANLTTDVVVNPGKSFMHIVTPTDNSEITTNTTKIRVRILNDTPTKVVYKVNDSNEEIPMTLDQDGYYSQDWSPSYQDNGKTAKITVIAYNGDSIEFEQSVNVFVKVPEILVKDYTFDTGIDGIQNNGTYPESMSLNIGHAVLAGDGKLEMTVTGMTYADSWQELKLQLTNIDDVLPYVNRVKFDVLIPATAASANPDATVRGIAMLPDDWDTKYGMTTTEKKITDLSTESIDGIQYAYFPVTIDLDSSKVSSAKGLAISVVGNGLNFDGTGEIYVDNIQLINAFVETPTDPSLVDDFESYQGNDAALQSKWIKASGDDISVSLTNDNAADGMYAMKVDYKLGSSGYAGVTKTLGGVDWSGYNKLKFYLVPDGSNQKLVIQIKVNGIYYEAYPSLSDSTPRWEEIGFNSFTVAPWDTQDQGKVITKEDLKNVQELSIYINDAGGSKSGTLYFDGIRAINDGTGGVPNGGSGSNSTPAQPGVLYDFENGTDGWTVDQNNANATATSITTDFASSGTHSLTSNFDLSKTDGFEIDKVQAIDLSAVKKISIDVKLSNGTATATLYIKTGSSWTWYDSGWQPINSGGFTTLSIDLDPSKINNLENVQSIGVKIVPDSGQTGNSNVYLDNVVLSN

SEQ ID NO: 23 - protein, 632 aa, Streptomyces sviceus
GTPTPVRIVDDEATPATRALFAYLKRQQGKGILFGHQHDLTYGFTFTTPNGRASDTRAGVGDYPAVFGWDTLILDGDERPGAEGASEAENIAALSRCIRQGDARGGINTLSAHMPNFVTGKNFHDTTGRVVSQILPGGAKHAHFNAFLDRIAKAVKRALRPDGTAIPVVFRPFHENNGAWFWWGAGHTTPAEFIELFRYTVEYLRDTRGVHNLLYAYSPNSSFAGDPADYLKTYPGDRFVDVLGFDSYDENAGPTPWLDAVVKDLAMVVRLANERGKAPAFTEFGEGGTEVRNQQWFTQLAQAIEADPLARQVTYMLTWANFGGTKRAYVPYPGHLLFPDFVKYEQDPYTLFAADLRGVYSAHTTAVKNAPFLHLVTPTDRQRVAAPRTTIRVRVTPARASQVTYSVNGGRARKLCLDTDGFYSGDWTIDPALRNNRSVTLTVSTRLDGKTLTDSAVVLLGELTPLPVGWVDDFEGYAADNTALSQAYTHVNSHTLTLSADHKSSGSYGLAYAYDFTGSEYTGAGKPLDADWSAFTSLALWLRGDGSANGGALQIVADGVDFWYQIPLSDTSGQDVRAPFSEFTPAPWDTEHTGAVLDAAHLAEVTAFNLYLVHGSGAATKGTVYVDNIRAE

SEQ ID NO: 24 - protein, 580 aa, Streptomyces scabiei
GTPTPVRIVDDRATPATRALFAYLRRQRGRGILFGHQHDLTYGFTFTTPDGRASDTRAAVGDYPAVFGWDTLVLDGDERPGTEDATDAENIAALSRCIRQGDARGGINTLSAHMPNFVTGKDFYDTRGRVVGQILPGGAKHARFNRFLDRVAKAVKGARRPDGTLIPVIFRPFHENNGGWFWWGAGHTTSGEFIELFRYTVEYLRDVKGVHNLLYAYSPNASLGGDPAAYLRTYPGDRFVDVLGYDSYDEGAGPTPWLDGLVRDLAMVVRLANERAYVPYPGHALLPDFVRFHQDPFTLFAADVRGVFAARTTAVRNGPSLHLVTPTDRQRVTAARTTVRVRVTPARASRVTYSVDGGPARRLRLDADGYHSGVWSIGPALRRKGSATLTVRARAGGETLTDSAVVLLGEAAPLPAGWIDDFEGYAGDDVALGEAYTHLNTHTLGLSREHKSSGSYGLAYAYDFTAAEFTGIGRPVVADWSAFTSLALWLRGDGSENAGALEIVADGIPFQYRFALDDTSGRELRAPFGEFGPAPWDTGNAGAVLDAARLAKVTGFNLYLGRASETVTKGVVYVDAVRAE

SEQ ID NO: 25 - DNA, 38 bp, Artificial Sequence
  synthetic primer
actagccgactagttcacaagaagggcgtcaacttaac

SEQ ID NO: 26 - DNA, 41 bp, Artificial Sequence
  synthetic primer
cttacgggctcgagttaacctaattcattgtggatatcacg

SEQ ID NO: 27 - DNA, 46 bp, Artificial Sequence
  synthetic primer
cttacgggctcgagttattggactttttctccactagaataataag

SEQ ID NO: 28 - DNA, 41 bp, Artificial Sequence
  synthetic primer
cttacgggctcgagttacccaaaataaccttcaaaatcatc

SEQ ID NO: 29 - DNA, 44 bp, Artificial Sequence
  synthetic primer
cttacgggctcgagttaaataggagcgacctctttttcctcttc

SEQ ID NO: 30 - DNA, 1053 bp, Bacillus sp.
tcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggttaa

SEQ ID NO: 31 - protein, 381 aa, Artificial Sequence
  synthetic Bsp Man4 sequence
MRSKKLWISLLFALTLIFTMAFSNMSAQATSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELG

SEQ ID NO: 32 - protein, 350 aa, Bacillus sp.
SQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELG

SEQ ID NO: 33 - DNA, 1428 bp, Bacillus sp.
tcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaataa

SEQ ID NO: 34 - protein, 506 aa, Artificial Sequence
  synthetic Bsp Man4 sequence
MRSKKLWISLLFALTLIFTMAFSNMSAQATSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQ

SEQ ID NO: 35 - protein, 475 aa, Bacillus sp.
SQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQ

SEQ ID NO: 36 - DNA, 2028 bp, Bacilllus sp.
tcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaagaagaaacaattcgtttatttgtaaaaattcctgaaatgtctttgttaacattaacgtttgatgatgatataaacggaatcaaaagcaatggaacatggcctgaagatggtgtaacatctgaaattgaccacgctattgtagatggagacggcaagttgatgttctctgttcaaggaatgtcacctactgaaacatggcaagagctcaagttagaattaacagaactatcagatgtgaacattgatgcggttaagaaaatgaagtttgacgcgcttatcccagcaggtagtgaagaaggttcagtccaaggaatcgtacaacttccaccggattgggagacgaaatatgggatgaatgaaacaacgaagtcaataaaagacttagagactgttactgttaatggaagcgattataaacggttggaagtgactgtttctatcgacaatcaaggaggagctacaggaatcgctttatcattagtaggatcccaactcgatttgttagaacctgtctacatcgataatattgaacttctaaattcctttgaagcaccaccagcagattcttttcttgttgatgattttgaaggttattttgggtaa

SEQ ID NO: 37 - protein, 706 aa, Artificial Sequence
  synthetic Bsp Man4 sequence
MRSKKLWISLLFALTLIFTMAFSNMSAQATSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFG

SEQ ID NO: 38 - protein, 675 aa, Bacillus sp.
SQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFG

SEQ ID NO: 39 - DNA, 2553 bp, Bacillus sp.
tcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaagaagaaacaattcgtttatttgtaaaaattcctgaaatgtctttgttaacattaacgtttgatgatgatataaacggaatcaaaagcaatggaacatggcctgaagatggtgtaacatctgaaattgaccacgctattgtagatggagacggcaagttgatgttctctgttcaaggaatgtcacctactgaaacatggcaagagctcaagttagaattaacagaactatcagatgtgaacattgatgcggttaagaaaatgaagtttgacgcgcttatcccagcaggtagtgaagaaggttcagtccaaggaatcgtacaacttccaccggattgggagacgaaatatgggatgaatgaaacaacgaagtcaataaaagacttagagactgttactgttaatggaagcgattataaacggttggaagtgactgtttctatcgacaatcaaggaggagctacaggaatcgctttatcattagtaggatcccaactcgatttgttagaacctgtctacatcgataatattgaacttctaaattcctttgaagcaccaccagcagattcttttcttgttgatgattttgaaggttattttggggatgacacgttgttacatcgcaattattctagcaatggagatccaattacactatcgttaacaagtgagtttaaaaataatggagaatttggattgaagtatgattattcgattggctcgatgggttatgcagggaggcaaacatcactaggacctgtcgattggagcggagctaatgcttttgaattttggatgaaacatggacaacttgaagggaatcatttaactgtacaaattcgaataggtgatgttagctttgaaaaaaatcttgaattaatggatgctcatgaaggtgtagtgacaatcccgttttctgaatttgctccagctgcttgggaaaataagcctggcgttatcattgacgaacaaaaattgaaaagagtgagtcaatttgctctttacacaggcggggctagacaatctggaacaatctactttgatgatttacgagcggtatatgatgaaagtttaccatcagttccagttccgaaagaggaggaagaggaaaaagaggtcgctcctatttaa

SEQ ID NO: 40 - protein, 881 aa, Artificial Sequence
  synthetic Bsp Man4 sequence
MRSKKLWISLLFALTLIFTMAFSNMSAQATSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKEVAPI

SEQ ID NO: 41 - protein, 850 aa, Bacillus sp.
SQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKEVAPI

SEQ ID NO: 42 - DNA, 1155 bp, Artificial Sequence
  synthetic aprE-Bsp Man4 sequence
gtgagaagcaaaaaattgtggatcagcttgttgtttgcgttaacgttaatctttacgatggcgttcagcaacatgagcgcgcaggcagctggtaaaactagttcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggttaa

SEQ ID NO: 43 - protein, 384 aa, Artificial Sequence
  synthetic aprE-Bsp Man4 sequence
VRSKKLWISLLFALTLIFTMAFSNMSAQAAGKTSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELG

SEQ ID NO: 44 - DNA, 1530 bp, Artificial Sequence
  synthetic aprE-Bsp Man4 sequence
gtgagaagcaaaaaattgtggatcagcttgttgtttgcgttaacgttaatctttacgatggcgttcagcaacatgagcgcgcaggcagctggtaaaactagttcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaataa

SEQ ID NO: 45 - protein, 509 aa, Artificial Sequence
  synthetic aprE-Bsp Man4 sequence
VRSKKLWISLLFALTLIFTMAFSNMSAQAAGKTSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQ

SEQ ID NO: 46 - DNA, 2130 bp, Artificial Sequence
  synthetic aprE-Bsp Man4 sequence
gtgagaagcaaaaaattgtggatcagcttgttgtttgcgttaacgttaatctttacgatggcgttcagcaacatgagcgcgcaggcagctggtaaaactagttcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaagaagaaacaattcgtttatttgtaaaaattcctgaaatgtctttgttaacattaacgtttgatgatgatataaacggaatcaaaagcaatggaacatggcctgaagatggtgtaacatctgaaattgaccacgctattgtagatggagacggcaagttgatgttctctgttcaaggaatgtcacctactgaaacatggcaagagctcaagttagaattaacagaactatcagatgtgaacattgatgcggttaagaaaatgaagtttgacgcgcttatcccagcaggtagtgaagaaggttcagtccaaggaatcgtacaacttccaccggattgggagacgaaatatgggatgaatgaaacaacgaagtcaataaaagacttagagactgttactgttaatggaagcgattataaacggttggaagtgactgtttctatcgacaatcaaggaggagctacaggaatcgctttatcattagtaggatcccaactcgatttgttagaacctgtctacatcgataatattgaacttctaaattcctttgaagcaccaccagcagattcttttcttgttgatgattttgaaggttattttgggtaa

SEQ ID NO: 47 - protein, 709 aa, Artificial Sequence
  synthetic aprE-Bsp Man4 sequence
VRSKKLWISLLFALTLIFTMAFSNMSAQAAGKTSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFG

SEQ ID NO: 48 - DNA, 2655 bp, Artificial Sequence
  synthetic aprE-Bsp Man4 sequence
gtgagaagcaaaaaattgtggatcagcttgttgtttgcgttaacgttaatctttacgatggcgttcagcaacatgagcgcgcaggcagctggtaaaactagttcacaagaagggcgtcaacttaacatggcagatgaggatgcttcaaagtatacgaaggagttatttgcttttcttcaagatgtaagtggttcacaagtgttatttggacaacagcatgcaacagatgaaggattaactttaacaaatccagctccaagaacaggttccactcaatctgaagttttcaatgcagttggggattatccagctgtgtttggatgggacacgaatagcctagatggtcgtgaaaagcctggcattgcaggtaatgtagaacaaagtataaaaaatacggctcagtccatgaaagtggctcatgatttaggagggattattacactaagcatgcacccagataattttgtaacagggggtccttatggtgatacaacagggaatgttgtaaaagaaattcttccaggtggatcaaaacatgcagagtttaacgcgtggttggacaatattgctgcgcttgctcacgagctgaaagatgagaatggtgaacctattccgatgatttttcggccattccatgaacaaacaggatcttggttttggtggggagcaagcacaacttcacccgaacaatataaagcgatttttcgttatacagtagaatatttgcgagatgttaaaggcgtaaataatattttatatggcttttcacctggggcgggacctgctggagatgtaaatcgctatttagaaacatatccaggggatgattacgttgatattttcggtattgacaattatgacaataaagacaatgcagggtcagaagcttggttaagtggtatggtcaaagacttggcgatgattagccgattagctgaacaaaaagaaaaagtagcggcttttactgagtatgggtacagtgcaaccggaattaatcgtcaagggaatacattagactggtacacacgtgtattagatgcgattgctgctgatgaagacgcacgtaaaatatcatacatgttgacatgggcgaactttggttggccgaataatatgtatgttccttatcgtgatatccacaatgaattaggtggagaccatgagttattaccggactttgaagctttccatgcggatgactacacagcatttcgagatgagataaaaggaaagatatataatactggaaaggaatataccgtttctcctcatgagccgtttatgtatgttatatctccgattacaggttctacagtgacaagcgaaacggtaacaatccaagcaaaagtagcgaatgacgaacacgcaagagtcactttcagggtcgatggttctagtttggaagaagaaatggttttcaatgatgacactttatattatacaggttcttttacaccagatgcagcagtgaatggcggagctgttgatgtgattgtagcttattattctagtggagaaaaagtccaagaagaaacaattcgtttatttgtaaaaattcctgaaatgtctttgttaacattaacgtttgatgatgatataaacggaatcaaaagcaatggaacatggcctgaagatggtgtaacatctgaaattgaccacgctattgtagatggagacggcaagttgatgttctctgttcaaggaatgtcacctactgaaacatggcaagagctcaagttagaattaacagaactatcagatgtgaacattgatgcggttaagaaaatgaagtttgacgcgcttatcccagcaggtagtgaagaaggttcagtccaaggaatcgtacaacttccaccggattgggagacgaaatatgggatgaatgaaacaacgaagtcaataaaagacttagagactgttactgttaatggaagcgattataaacggttggaagtgactgtttctatcgacaatcaaggaggagctacaggaatcgctttatcattagtaggatcccaactcgatttgttagaacctgtctacatcgataatattgaacttctaaattcctttgaagcaccaccagcagattcttttcttgttgatgattttgaaggttattttggggatgacacgttgttacatcgcaattattctagcaatggagatccaattacactatcgttaacaagtgagtttaaaaataatggagaatttggattgaagtatgattattcgattggctcgatgggttatgcagggaggcaaacatcactaggacctgtcgattggagcggagctaatgcttttgaattttggatgaaacatggacaacttgaagggaatcatttaactgtacaaattcgaataggtgatgttagctttgaaaaaaatcttgaattaatggatgctcatgaaggtgtagtgacaatcccgttttctgaatttgctccagctgcttgggaaaataagcctggcgttatcattgacgaacaaaaattgaaaagagtgagtcaatttgctctttacacaggcggggctagacaatctggaacaatctactttgatgatttacgagcggtatatgatgaaagtttaccatcagttccagttccgaaagaggaggaagaggaaaaagaggtcgctcctatttaa

SEQ ID NO: 49 - protein, 884 aa, Artificial Sequence
  synthetic aprE-Bsp Man4 sequence
VRSKKLWISLLFALTLIFTMAFSNMSAQAAGKTSSQEGRQLNMADEDASKYTKELFAFLQDVSGSQVLFGQQHATDEGLTLTNPAPRTGSTQSEVFNAVGDYPAVFGWDTNSLDGREKPGIAGNVEQSIKNTAQSMKVAHDLGGIITLSMHPDNFVTGGPYGDTTGNVVKEILPGGSKHAEFNAWLDNIAALAHELKDENGEPIPMIFRPFHEQTGSWFWWGASTTSPEQYKAIFRYTVEYLRDVKGVNNILYGFSPGAGPAGDVNRYLETYPGDDYVDIFGIDNYDNKDNAGSEAWLSGMVKDLAMISRLAEQKEKVAAFTEYGYSATGINRQGNTLDWYTRVLDAIAADEDARKISYMLTWANFGWPNNMYVPYRDIHNELGGDHELLPDFEAFHADDYTAFRDEIKGKIYNTGKEYTVSPHEPFMYVISPITGSTVTSETVTIQAKVANDEHARVTFRVDGSSLEEEMVFNDDTLYYTGSFTPDAAVNGGAVDVIVAYYSSGEKVQEETIRLFVKIPEMSLLTLTFDDDINGIKSNGTWPEDGVTSEIDHAIVDGDGKLMFSVQGMSPTETWQELKLELTELSDVNIDAVKKMKFDALIPAGSEEGSVQGIVQLPPDWETKYGMNETTKSIKDLETVTVNGSDYKRLEVTVSIDNQGGATGIALSLVGSQLDLLEPVYIDNIELLNSFEAPPADSFLVDDFEGYFGDDTLLHRNYSSNGDPITLSLTSEFKNNGEFGLKYDYSIGSMGYAGRQTSLGPVDWSGANAFEFWMKHGQLEGNHLTVQIRIGDVSFEKNLELMDAHEGVVTIPFSEFAPAAWENKPGVIIDEQKLKRVSQFALYTGGARQSGTIYFDDLRAVYDESLPSVPVPKEEEEEKEVAPI